(19) United States
(12) Patent Application Publication
(10) Pub. No.: US 2011/0135572 A1
(43) Pub. Date: Jun. 9, 2011

(54) COMPOSITIONS AND METHODS FOR DIAGNOSING LATE-ONSET ALZHEIMER'S DISEASE

(75) Inventor: Shirley Poduslo, North Augusta, SC (US)

(73) Assignee: Medical College of Georgia Research Institute, Inc. Georgia

(21) Appl. No.: 12/790,045

(22) Filed: May 28, 2010

Related U.S. Application Data
(63) Continuation of application No. PCT/US2009/042837, filed on May 5, 2009.
(60) Provisional application No. 61/126,521, filed on May 5, 2008.

(51) Int. Cl. C12Q 1/68; A61K 49/00; C40B 40/06

(52) U.S. Cl. 424/9.1; 435/6.11; 506/16

(57) ABSTRACT

Genetic markers for late-onset Alzheimer's disease (AD) within the TRPC4AP gene locus are provided. The genetic markers include single nucleotide polymorphisms (SNPs) and haplotypes. Methods and kits for using the disclosed genetic markers to identify, or assist in the identification of, subjects as having late-onset AD, or as having an increased risk for developing late-onset AD are provided. In a preferred embodiment the genetic markers are one or more SNPs selected from the group consisting of rs1058003, rs3746430, rs3736802, rs6088677, rs6087660, rs4911460, rs6087664, rs13042358, rs6088692, rs6120816, rs1885119, rs2065108 and rs6088727, or haplotype: rs1058003_G: rs3746430_T: rs3736802_T: rs6088677_C: rs6087660_T: rs4911460_G: rs6087664_C: rs13042358_G: rs6120816_G: rs1885119_T.

### CROSS REFERENCE TO RELATED APPLICATIONS

This application claims priority to and benefit of U.S. Provisional Patent Application No. 61/126,521, filed on May 5, 2008, by Shirley E. Poduslo, and where permissible is incorporated by reference in its entirety.

### STATEMENT REGARDING FEDERALLY SPONSORED RESEARCH OR DEVELOPMENT

This invention was made with Government Support under Cooperative Agreement Grant No. U2AG21886 from the National Institute of Aging for the National Cell Repository. The Government has certain rights in the invention.

### FIELD OF THE INVENTION

The present disclosure generally relates to the field of biomarkers for late-onset Alzheimer's disease.

### BACKGROUND OF THE INVENTION

Alzheimer's disease (AD) is the most significant and common cause of dementia in developed countries, accounting for 60% or more of all cases of dementia. AD is a progressive neurodegenerative disorder characterized clinically by memory loss of subtle onset, followed by a slowly progressive dementia that has a course of several years. Brain pathology of AD is characterized by gross, diffuse atrophy of the cerebral cortex with secondary enlargement of the ventricular system. Microscopically, there are neuritic plaques containing AD amyloid, silver-staining neurofibrillary tangles in neuronal cytoplasm, and accumulation of AD amyloid in arterial walls of cerebral blood vessels. A definite diagnosis of AD can only occur at autopsy, where the presence of amyloid plaques and neurofibrillary tangles is confirmed.

The frequency of AD increases with each decade of adult life, reaching 20-40% of the population over the age of 85. Because more and more people will live into their 80's and 90's, the number of patients is expected to triple over the next 20 years. More than 4 million people suffer from AD in the USA, where 800,000 deaths per year are associated with AD. It is estimated that the cost of AD in the USA is $80 billion to $100 billion a year in medical care, personal caretaking and lost productivity. AD also puts a heavy emotional toll on family members and caregivers: about 2.7 million people care for AD patients in the USA. AD patients live for an average of 7 to 10 years after diagnosis and spend an average of 5 years under care either at home or in a nursing home.

AD is presumed to have a genetic component, as evidenced by an increased risk for AD among first degree relatives of affected individuals. Based on twin studies, heritability for the disease has been estimated at 79%, with no difference (after controlling for age) between men and women in prevalence or heritability (Gatz, et al., Arch. Gen. Psychiatry, 63:168-74 (2006)).

So far, three genes have been identified in patients with early onset AD that lead to the less common, dominantly inherited form of dementia. Mutations in the three genes, beta-amyloid precursor protein (Goate, et al., Nature, 349:704-706 (1991)), presenilin 1 (Sherrington, et al., Nature, 375:754-760 (1995)), and presenilin 2 (Levy-Lahad, et al., Science, 269:973-977 (1996)) lead to an increase in the production of Aβ42, the main component in amyloid plaques. Although early onset AD makes up less than 5% of all AD cases, the identification of these genes has contributed substantially to the understanding of the disease process.

Late-onset Alzheimer's disease (LOAD) is a much more common form of this dementia characterized by cognitive decline and distinct neuropathology. Early genetic studies of AD demonstrated association and linkage to the same region on chromosome 19 containing the APOE gene (Schellenberg, et al., J. Neurogenet., 4:97-108 (1987); Pericak-Vance, et al., Am. J. Hum. Gen., 48:1034-1050 (1991)). Three common alleles were identified for the APOE gene, ε2, ε3, ε4. The ε4 allele frequency is increased to 50% in affected individuals versus 14% in controls (Corder, et al., Science, 281:921-923 (1993)). Although there is strong association between AD and the APOE-ε4 allele, which has been confirmed in many studies, most investigators consider the APOE ε4 allele to be neither necessary nor sufficient for the development of AD. APOE is considered a major risk factor, but APOE testing does not provide enough sensitivity and specificity for use as an independent diagnostic test and therefore is not recommended as a diagnostic marker for the prediction of AD (National Institute on Aging/Alzheimer's Association Working Group, 1996). Collectively, mutations in beta-amyloid precursor protein, presenilin-1, presenilin-2 and APOE genes account for less than 25% of the disease prevalence.

Despite the high prevalence of AD today and its expected prevalence increase in an aging population, there are currently no diagnostic tests available that determine the cause of dementia and adequately differentiate between AD and other types of dementias. A diagnostic test that, for example, enables physicians to identify AD early in the disease process, or identify individuals who are at high risk of developing the disease, will provide the option to intervene at an early stage in the disease process. Early intervention in disease processes does generally result in better treatment results by delaying disease onset or progression compared to later intervention.

Thus, there is a need for diagnostic markers that enable the detection of AD at an early stage of the disease or that identify individuals who are at high risk of developing AD.

Therefore, it is an object of the invention to provide genetic markers indicative of late-onset AD, and methods for using the genetic markers for the diagnosis of subjects that have late-onset AD, or that have an increased risk for developing late-onset AD.

It is another object of the invention to provide methods for using the genetic markers for pharmacogenomic evaluation of a subject to determine which therapeutic or prophylactic strategy is most likely to be effective in the subject.

### SUMMARY OF THE INVENTION

Genetic markers for late-onset Alzheimer's disease (AD) are provided. The genetic markers include alterations in the gene locus for transient receptor potential cation channel, subfamily C, member 4 associated protein (TRPC4AP). Suitable alterations include, but are not limited to, polymorphisms, mutations, deletions, rearrangements, and/or insertions in the coding and/or non-coding regions of the TRPC4AP gene.

In some embodiments, the genetic markers include one or more single nucleotide polymorphisms (SNPs) within the TRPC4AP gene locus. The genetic markers include one or more of the following single nucleotide polymorphisms (SNPs) in any combination, referred to by their dbSNP Database RS ID numbers as: rs1058003, rs3746430, rs3736802, rs6088677, rs6087660, rs4911460, rs6087664, rs13042358, rs6088692; rs6120816 and rs1885119. In preferred embodiments, the genetic markers include one or more of the following alleles of the above-listed SNPs in any combination: a ‘G’ allele at SNP rs1058003; a ‘T’ allele at rs3746430; a ‘T’ allele at rs3736802; a ‘C’ allele at rs6088677; a ‘T’ allele at rs6087660; a ‘G’ allele at rs4911460; a ‘C’ allele at rs6087664; a ‘G’ allele at rs13042358; a ‘G’ allele at rs6120816 and a ‘T’ allele at rs1885119.

In another embodiment, the genetic marker is a haplotype that includes two or more of the above-referenced SNPs in any combination. In a preferred embodiment, the haplotype is rs1058003: rs3746430: rs3736802: rs6088677: rs6087660: rs4911460: rs6087664: rs13042358: rs6120816: rs1885119: G:T:T:C:T:G:C:G:G:T. This haplotype can also be expressed as rs1058003_G: rs3746430_T: rs3736802_T: rs6088677_C: rs6087660_T: rs4911460_G: rs6087664_C: rs13042358_G: rs6120816_G: rs1885119_T. In a particularly preferred embodiment, the genotype for each SNP of this haplotype is homozygous.

Methods for using the disclosed genetic markers to identify, or assist in the identification of subjects having late-onset AD, or having an increased risk for developing late-onset AD are provided. The methods include the steps of obtaining a biological sample containing genomic nucleic acids from the subject and detecting the presence or absence of one or more of the disclosed genetic markers in the biological sample. The detecting step can include determining whether or not the subject is heterozygous or homozygous for the genetic marker.

Detection of the disclosed genetic markers can be used in combination with one or more additional diagnostic approaches for identifying subjects with or at increased risk of developing late-onset AD. Suitable diagnostic methods include, but are not limited to mental status exams, imaging procedures, and the detection of additional genetic markers.

Kits and systems for detecting the disclosed genetic markers are also provided. The kits can include packaged probe and primer sets, arrays of nucleic acid molecules, or beads that contain one or more probes, primers, or other detection reagents. The kits may additionally contain other components necessary to carry out a reaction or assay. In other embodiments, the kits are compartmentalized kits which contain reagents in separate containers.

Methods for using the disclosed genetic markers for pharmacogenomic evaluation to determine therapeutic or prophylactic strategies likely to be effective in treating a subject are also provided.

Also provided are methods for using the disclosed genetic markers as research tools to identify additional genetic markers for late-onset AD using linkage disequilibrium analysis.

### BRIEF DESCRIPTION OF THE DRAWINGS

FIG. 1 is a circular pedigree chart showing the pedigree for extended Family 1.

FIG. 2 is a circular pedigree chart showing the pedigree for extended Family 2.

### DETAILED DESCRIPTION OF THE INVENTION

### I. Definitions

As used herein, the term “allele” refers to one of a pair or series, of forms of a gene or non-genic region that occur at a given locus in a chromosome. In a normal diploid cell there are two alleles of any one gene (one from each parent), which occupy the same relative position (locus) on homologous chromosomes. Within a population there may be more than two alleles of a gene. SNPs also have alleles, i.e., the two (or more) nucleotides that characterize the SNP.

As used herein, the term “linkage disequilibrium” or “LD” refers to the situation in which the alleles for two or more loci do not occur together in individuals sampled from a population at frequencies predicted by the product of their individual allele frequencies. Markers that are in LD do not follow Mendel's second law of independent random segregation. LD can be caused by any of several demographic or population artifacts as well as by the presence of genetic linkage between markers. However, when these artifacts are controlled and eliminated as sources of LD, then LD results directly from the fact that the loci involved are located close to each other on the same chromosome so that specific combinations of alleles for different markers (haplotypes) are inherited together. Markers that are in high LD can be assumed to be located near each other and a marker or haplotype that is in high LD with a genetic trait can be assumed to be located near the gene that affects that trait.

As used herein, the term “locus” refers to a specific position along a chromosome or DNA sequence. Depending upon context, a locus could be a gene, a marker, a chromosomal band or a specific sequence of one or more nucleotides.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

As used herein, the term “genotype” refers to a set of alleles at a specified locus or loci.

As used herein, the term “single nucleotide polymorphism (SNP)” refers to a variation of a single nucleotide. This includes the replacement of one nucleotide by another and deletion or insertion of a single nucleotide. Typically, SNPs are bi-allelic markers although tri- and tetra-allelic markers also exist. For example, SNP AC may include allele C or allele A. Thus, a nucleic acid molecule having SNP AC may include a C or A at the polymorphic position.

As used herein, the term “haplotype” refers to the allelic pattern of a group of (usually contiguous) DNA markers or other polymorphic loci along an individual chromosome or double helical DNA segment. Haplotypes identify individual chromosomes or chromosome segments. The presence of shared haplotype patterns among a group of individuals implies that the locus defined by the haplotype has been inherited, identical by descent (IBD), from a common ancestor. In some instances, a specific allele or haplotype may be associated with susceptibility to a disorder or condition of interest, e.g., late-onset Alzheimer's disease. The term “haplotype” is specifically used herein to refer to a combination of SNP alleles, e.g., the alleles of the SNPs found together on a single DNA molecule. In specific embodiments, the SNPs in a haplotype are in linkage disequilibrium with one another.

As used herein the term “isolated” is meant to describe a compound of interest (e.g., nucleic acids) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. Isolated nucleic acids are at least 60% free, preferably 75% free, and most preferably 90% free from other associated components.

As used herein, a “genetic marker” is an identifiable DNA sequence that is variable (polymorphic) for different individuals within a population. Exemplary genetic markers include SNPs and haplotypes.

As used herein, the terms “probe” or “primer” refer to a nucleic acid or oligonucleotide that forms a hybrid structure with a sequence in a target region of a nucleic acid due to complementarity of the probe or primer sequence to at least one portion of the target region sequence.

The terms “individual”, “host”, “subject”, and “patient” are used interchangeably herein, and refer to a mammal, including, but not limited to, humans, rodents such as mice and rats, and other laboratory animals.

### II. Genetic Markers for Late-Onset Alzheimer's Disease

Genetic markers for late-onset Alzheimer's disease (AD) are provided. It has been discovered that alterations in the gene locus for transient receptor potential cation channel, subfamily C, member 4 associated protein (TRPC4AP) are associated with the development of late-onset AD.

A. TRPC4AP

The transient receptor potential (TRP) cation channels are part of a superfamily of 28 channels subdivided into six subfamilies. Most of the channels provide entry for calcium ions which are involved in the regulation of many calcium-dependent cell functions. Dysfunctions of the channels are thought to cause human disease or contribute to the progression of the disease (Nilius, Biochim. Biophys. Acta, 1772:804-812 (2007)).

The examples below demonstrate that alterations in the gene locus for TRPC4AP are associated with the development of late-onset AD. The gene encoding TRPC4AP or tumor necrosis factor receptor-associated ubiquitous scaffolding and signaling protein (TRUSS) is located on chromosome 20q11.22, contains 19 exons and has a length of 90,411 bases. The gene is located between positions 33,053,868 and 33,144,279 as read from the reverse coding strand. The forward strand of the TRPC4AP gene locus is provided as SEQ ID NO:1. The sequence of the TRPC4AP gene is also provided by GenBank Accession No. NC_{—}000020 and Entrez GeneID Accession No. 26133.

The gene produces two alternative transcripts, referred to as isoform 1 and isoform 2. In a preferred embodiment, the genetic markers are contained within isoform 1. The mRNA sequence for TRPC4AP variant 1 is provided by GenBank Accession No. NM_{—}015638. The mRNA sequence for TRPC4AP variant 2 is provided by GenBank Accession No. NM_{—}199368. There are at least 17 spliced variants listed in GenBank, and thus, the gene locus may produce several proteins. According to AceView, there may be 20 different mRNAs.

One known protein which is encoded by mouse TRUSS has 797 amino acids with a mass of 90,852 Da. The protein is expressed in heart, liver, testis, and brain (Soond, et al., Mol Cell Biol., 23:8334:8344 (2003)). The protein interacts with TNF-R1 (the tumor necrosis factor receptor 1), making the complex insensitive to stimulation with TNF-a. In addition, the protein may be involved with the activation of transcription factors such as NF-κB and may serve as a scaffolding protein that links TNF-R1 to components of the IkB-kinase complex (Soond, et al., FEBS Lett., 580:4591-4596 (2006)). The protein may also function in the TNF-a induced Jun NH2-terminal kinases (JNK) and the transcription factor (AP-1) activation (Soond, et al., FEBS Lett., 580:4591-4596 (2006)). TNF is a proinflammatory cytokine which may be involved with the pathology of Alzheimer's disease. The neurotoxicity in Alzheimer's disease may indeed be mediated by inflammatory processes in the brain; proinflammatory cytokines, such as TNF-a, may be released from activated microglia which could lead to the neuronal apoptosis found in the disease process. The protein may also have a MHC class 1 binding function (Antoniou, et al., Immunology, 106:182-189 (2002)).

B. TRPC4AP Gene Alterations

The disclosed genetic markers for late-onset AD include alterations in the gene locus for TRPC4AP. Suitable alterations include, but are not limited to polymorphisms, mutations, deletions, rearrangements, and/or insertions in the coding and/or non-coding regions of the TRPC4AP, alone or in combination.

Mutations more specifically include point mutations. Deletions may encompass any region of two or more residues in a coding or non-coding portion of the gene locus, such as from two residues up to the entire gene or locus. Typical deletions affect smaller regions, such as domains (introns) or repeated sequences or fragments of less than about 50 consecutive base pairs, although larger deletions may occur as well. Insertions may encompass the addition of one or several residues in a coding or non-coding portion of the gene locus. Insertions may typically comprise an addition of between 1 and 50 base pairs in the gene locus. Rearrangement includes inversion of sequences. The TRPC4AP gene locus alteration may result in the creation of stop codons, frameshift mutations, amino acid substitutions, particular RNA splicing or processing, product instability, truncated polypeptide production, etc. The alteration may result in the production of a TRPC4AP polypeptide with altered function, stability, targeting or structure. The alteration may also cause a reduction or an increase in protein expression. The alteration may be determined at the level of the TRPC4AP DNA, RNA or polypeptide.

1. Single Nucleotide Polymorphisms

In some embodiments, the genetic markers include one or more single nucleotide polymorphisms (SNPs) within the TRPC4AP gene locus. SNPs are single base positions in DNA at which different alleles, or alternative nucleotides exist in a population. Approximately 90% of all polymorphisms in the human genome are SNPs. The SNP position is usually preceded by and followed by highly conserved sequences of the allele (e.g., sequences that vary in less than 1/100 or 1/1000 members of the populations). An individual may be homozygous or heterozygous for an allele at each SNP position.

A SNP may arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP may also be a single base insertion or deletion variant referred to as an “indel” (Weber, et al., Am. J. Hum. Genet., 71 (4):854-62 (2002)).

In certain embodiments, the genetic markers include one or more of the following SNPs in any combination, referred to by their dbSNP Database RS ID numbers: rs1058003, rs3746430, rs3736802, rs6088677, rs6087660, rs4911460, rs6087664, rs13042358, rs6088692; rs6120816; rs1885119; rs2065108 and rs6088727. These SNPs are listed in order as read from the forward strand and not the reverse coding strand, and are located at the following physical positions on chromosome 20, respectively: 33,054,078; 33,057,335; 33,067,703; 33,071,125; 33,080,189; 33,087,991; 33,089,877; 33,098,140; 33,102,249; 33,108,019; 33,109,310; 33,170,483 and 33,177,300. These positions correspond to positions 210; 3,467; 13,835; 17,257; 26,321; 34,123; 36,009; 44,272; 48,381; 54,151; 55,442; 116,615 and 123,432 of SEQ ID NO:1, respectively.

In preferred embodiments, the genetic markers include one or more of the following alleles of the above-listed SNPs in any combination: a ‘G’ allele at SNP rs1058003; a ‘T’ allele at rs3746430; a ‘T’ allele at rs3736802; a ‘C’ allele at rs6088677; a ‘T’ allele at rs6087660; a ‘G’ allele at rs4911460; a ‘C’ allele at rs6087664; a ‘G’ allele at rs13042358; a ‘G’ allele at rs6120816 and a ‘T’ allele at rs1885119.

2. Haplotypes

In other embodiments, the genetic marker is a haplotype that includes two or more of the above-referenced SNPs in any combination. In a preferred embodiment, the haplotype is rs1058003: rs3746430: rs3736802: rs6088677: rs6087660: rs4911460: rs6087664: rs13042358: rs6120816: rs1885119: G:T:T:C:T:G:C:G:G:T. This haplotype can also be expressed as rs1058003_G: rs3746430_T: rs3736802_T: rs6088677_C: rs6087660_T: rs4911460_G: rs6087664_C: rs13042358_G: rs6120816_G: rs1885119_T. This haplotype is referred to herein as the “H1 haplotype”. In a particularly preferred embodiment, the genotype for each SNP of the H1 haplotype is homozygous.

### III. Methods for Using Genetic Markers for Late-Onset Alzheimer's Disease

### A. Diagnosis

The disclosed genetic markers can be used to identify, or assist in the identification of subjects having late-onset AD or having an increased risk of developing late-onset AD. A subject identified as having an increased risk of developing late-onset AD is a subject whose level of risk of developing late-onset AD is greater than the level of risk of a subject lacking the disclosed genetic markers.

Methods of diagnosing a subject as having late-onset AD or as having an increased risk of developing late-onset AD include the steps of obtaining a biological sample containing nucleic acid from the subject and detecting the presence or absence of one or more of the disclosed genetic markers in the biological sample. Any biological sample that contains the DNA of the subject to be diagnosed can be employed, including tissue samples and blood samples, with nucleated blood cells being a particularly convenient source. The DNA may be isolated from the biological sample prior to testing the DNA for the presence or absence of the disclosed genetic markers. Methods for detecting the disclosed genetic markers are provided below.

In one embodiment, the DNA of the biological sample is tested for the presence or absence of one or more of the following SNPs in any combination: rs1058003, rs3746430, rs3736802, rs6088677, rs6087660, rs4911460, rs6087664, rs13042358, rs6088692; rs6120816; rs1885119; rs2065108 and rs6088727, where the presence of one or more of these SNPs is indicative that the subject has, or is at increased risk of developing, late-onset AD. For example, the DNA may be tested for the presence or absence of any combination of 1, 2, 3, 4, 5, 6, 7, 8, 9, 10, 11, 12, or all 13 of these SNPs.

In other embodiments, the DNA of the biological sample is tested for the presence or absence of one or more of the following alleles in any combination: a ‘G’ allele at SNP rs1058003; a ‘T’ allele at rs3746430; a ‘T’ allele at rs3736802; a ‘C’ allele at rs6088677; a ‘T’ allele at rs6087660; a ‘G’ allele at rs4911460; a ‘C’ allele at rs6087664; a ‘G’ allele at rs13042358; a ‘G’ allele at rs6120816 and a ‘T’ allele at rs1885119, where the presence of one or more of these alleles is indicative that the subject has, or is at increased risk of developing, late-onset AD. For example, the DNA may be tested for the presence or absence of any combination of 1, 2, 3, 4, 5, 6, 7, 8, 9, or all 10 of these alleles.

In another embodiment, the DNA of the biological sample is tested for the presence or absence of the H1 haplotype (rs1058003_G: rs3746430_T: rs3736802_T: rs6088677_C: rs6087660_T: rs4911460_G: rs6087664_C: rs13042358_G: rs6120816_G: rs1885119_T), where the presence of the haplotype is indicative that the subject has, or is at increased risk of developing, late-onset AD.

The detecting step can include determining whether the subject is heterozygous or homozygous for the genetic marker. The step of detecting the presence or absence of the genetic marker can include the step of detecting the presence or absence of the marker in both chromosomes of the subject (i.e., detecting the presence or absence of one or two alleles containing the marker or functional polymorphism). More than one copy of a genetic marker (i.e., subjects homozygous for the genetic marker) can indicate a greater risk of developing late-onset AD, or can provide greater confidence in the diagnosis of a subject having late-onset AD.

### B. Methods for Detecting SNPs and Haplotypes

The process of determining which specific nucleotide (i.e., allele) is present at each of one or more SNP positions, such as a disclosed SNP position in the TRPC4AP gene locus, is referred to as SNP genotyping. Methods for SNP genotyping are generally known in the art (Chen et al., Pharmacogenomics J., 3(2):77-96 (2003); Kwok, et al., Curr. Issues Mol. Biol., 5(2):43-60 (2003); Shi, Am. J. Pharmacogenomics, 2(3):197-205 (2002); and Kwok, Annu. Rev. Genomics Hum. Genet., 2:235-58 (2001)).

SNP genotyping can include the steps of collecting a biological sample from a subject (e.g., sample of tissues, cells, fluids, secretions, etc.), isolating genomic DNA from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

The neighboring sequence can be used to design SNP detection reagents such as oligonucleotide probes and primers. Exemplary primers for the TRPC4AP gene are provided in Table 4. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

SNPs can be scored by direct DNA sequencing. A variety of automated sequencing procedures can be utilized, including sequencing by mass spectrometry. Methods for amplifying DNA fragments and sequencing them are well known in the art.

Other suitable methods for detecting polymorphisms include methods in which protection from cleavage agents is used to detect mismatched bases in RNA/RNA or RNA/DNA duplexes (Myers et al., Science, 230:1242 (1985); Cotton, et al., PNAS, 85:4397 (1988); and Saleeba, et al., Meth. Enzymol., 217:286-295 (1992)), comparison of the electrophoretic mobility of variant and wild type nucleic acid molecules (Orita et al., PNAS, 86:2766 (1989); Cotton, et al, Mutat. Res., 285:125-144 (1993); and Hayashi, et al., Genet. Anal. Tech. Appl., 9:73-79 (1992)), and assaying the movement of polymorphic or wild-type fragments in polyacrylamide gels containing a gradient of denaturant using denaturing gradient gel electrophoresis (DGGE) (Myers et al., Nature, 313:495 (1985)). Sequence variations at specific locations can also be assessed by nuclease protection assays such as RNase and S1 protection or chemical cleavage methods.

In one embodiment, SNP genotyping is performed using the TaqMan® assay, which is also known as the 5′ nuclease assay. The TaqMan® assay detects the accumulation of a specific amplified product during PCR. The TaqMan® assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′-most and the 3′-most ends, respectively, or vice versa. Alternatively, the reporter dye may be at the 5′- or 3′-most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

Another method for genotyping SNPs is the use of two oligonucleotide probes in an OLA (U.S. Pat. No. 4,988,617). In this method, one probe hybridizes to a segment of a target nucleic acid with its 3′-most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization-Time of Flight) mass spectrometry technology is useful for extremely precise determinations of molecular mass, such as SNPs. Numerous approaches to SNP analysis have been developed based on mass spectrometry. Exemplary mass spectrometry-based methods of SNP genotyping include primer extension assays, which can also be utilized in combination with other approaches, such as traditional gel-based formats and microarrays.

Typically, the primer extension assay involves designing and annealing a primer to a template PCR amplicon upstream (5′) from a target SNP position. A mix of dideoxynucleotide triphosphates (ddNTPs) and/or deoxynucleotide triphosphates (dNTPs) are added to a reaction mixture containing template (e.g., a SNP-containing nucleic acid molecule which has typically been amplified, such as by PCR), primer, and DNA polymerase. Extension of the primer terminates at the first position in the template where a nucleotide complementary to one of the ddNTPs in the mix occurs. The primer can be either immediately adjacent (i.e., the nucleotide at the 3′ end of the primer hybridizes to the nucleotide next to the target SNP site) or two or more nucleotides removed from the SNP position. If the primer is several nucleotides removed from the target SNP position, the only limitation is that the template sequence between the 3′ end of the primer and the SNP position cannot contain a nucleotide of the same type as the one to be detected, or this will cause premature termination of the extension primer. Alternatively, if all four ddNTPs alone, with no dNTPs, are added to the reaction mixture, the primer will always be extended by only one nucleotide, corresponding to the target SNP position. In this instance, primers are designed to bind one nucleotide upstream from the SNP position (i.e., the nucleotide at the 3′ end of the primer hybridizes to the nucleotide that is immediately adjacent to the target SNP site on the 5′ side of the target SNP site). Extension by only one nucleotide is preferable, as it minimizes the overall mass of the extended primer, thereby increasing the resolution of mass differences between alternative SNP nucleotides. Furthermore, mass-tagged ddNTPs can be employed in the primer extension reactions in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer. The extended primers can then be purified and analyzed by MALDI-TOF mass spectrometry to determine the identity of the nucleotide present at the target SNP position.

Other methods that can be used to genotype the SNPs include single-strand conformational polymorphism (SSCP), and denaturing gradient gel electrophoresis (DGGE). SSCP identifies base differences by alteration in electrophoretic migration of single stranded PCR products. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel.

Sequence-specific ribozymes (U.S. Pat. No. 5,498,531) can also be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis.

### C. SNP Detection Kits

Detection reagents can be developed and used to assay the disclosed SNPs individually or in combination, and such detection reagents can be readily incorporated into a kit or system format. The terms “kits” and “systems”, as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.). SNP detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more of the disclosed SNPs are provided. The kits/systems can optionally include various electronic hardware components; for example, arrays (“DNA chips”) and microfluidic systems (“lab-on-a-chip” systems) provided by various manufacturers typically comprise hardware components. Other kits/systems (e.g., probe/primer sets) may not include electronic hardware components, but may be comprised of, for example, one or more SNP detection reagents (along with, optionally, other biochemical reagents) packaged in one or more containers.

In some embodiments, a SNP detection kit typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A kit may further contain means for determining the amount of a target nucleic acid, and means for comparing the amount with a standard, and can comprise instructions for using the kit to detect the SNP-containing nucleic acid molecule of interest. In one embodiment, kits are provided which contain the necessary reagents to carry out one or more assays to detect one or more of the disclosed SNPs. In an exemplary embodiment, SNP detection kits/systems are in the form of nucleic acid arrays, or compartmentalized kits, including microfluidic/lab-on-a-chip systems.

SNP detection kits may contain, for example, one or more probes, or pairs of probes, that hybridize to a nucleic acid molecule at or near each target SNP position. Exemplary primers are provided in Table 3 and Table 4 below. Multiple pairs of allele-specific probes may be included in the kit/system to simultaneously assay large numbers of SNPs. In some kits, the allele-specific probes are immobilized to a substrate such as an array or bead.

The terms “arrays”, “microarrays”, and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate.

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized. Probes can be attached to a solid support in an ordered, addressable array.

A microarray can be composed of a large number of unique, single-stranded polynucleotides, usually either synthetic antisense polynucleotides or fragments of cDNAs, fixed to a solid support. Typical polynucleotides are about 6-60 nucleotides in length, or about 15-30 nucleotides in length, or about 18-25 nucleotides in length. For certain types of microarrays or other detection kits/systems, it may be preferable to use oligonucleotides that are only about 7-20 nucleotides in length. In other types of arrays, such as arrays used in conjunction with chemiluminescent detection technology, exemplary probe lengths can be, for example, about 15-80 nucleotides in length, or about 50-70 nucleotides in length, or about 55-65 nucleotides in length, or about 60 nucleotides in length. The microarray or detection kit can contain polynucleotides that cover the known 5′ or 3′ sequence of a gene/transcript or target SNP site, sequential polynucleotides that cover the full-length sequence of a gene/transcript; or unique polynucleotides selected from particular are as along the length of a target gene/transcript sequence. Polynucleotides used in the microarray or detection kit can be specific to a SNP or SNPs of interest (e.g., specific to a particular SNP allele at a target SNP site, or specific to particular SNP alleles at multiple different SNP sites).

Hybridization assays based on polynucleotide arrays rely on the differences in hybridization stability of the probes to perfectly matched and mismatched target sequence variants. For SNP genotyping, it is generally preferable that stringency conditions used in hybridization assays are high enough such that nucleic acid molecules that differ from one another at as little as a single SNP position can be differentiated. Such high stringency conditions may be preferable when using, for example, nucleic acid arrays of allele-specific probes for SNP detection. In some embodiments, the arrays are used in conjunction with chemiluminescent detection technology.

A polynucleotide probe can be synthesized on the surface of the substrate by using a chemical coupling procedure and an inkjet application apparatus, as described in PCT Publication No. WO 95/251116. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures.

Methods for using such arrays or other kits/systems, to identify SNPs and haplotypes disclosed herein in a test sample are provided. Such methods typically involve incubating a test sample of nucleic acids with an array comprising one or more probes corresponding to at least one SNP position of the present invention, and assaying for binding of a nucleic acid from the test sample with one or more of the probes. Conditions for incubating a SNP detection reagent (or a kit/system that employs one or more such SNP detection reagents) with a test sample vary. Incubation conditions depend on such factors as the format employed in the assay, the detection methods employed, and the type and nature of the detection reagents used in the assay.

A SNP detection kit/system can include components that are used to prepare nucleic acids from a test sample for the subsequent amplification and/or detection of a SNP-containing nucleic acid molecule. Such sample preparation components can be used to produce nucleic acid extracts (including DNA and/or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue specimens.

Another form of kit is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other SNP detection reagent for detecting one or more of the disclosed SNPs, one or more containers which contain wash reagents (such as phosphate buffered saline, Tris-buffers, etc.), and one or more containers which contain the reagents used to reveal the presence of the bound probe or other SNP detection reagents. The kit can optionally further include compartments and/or reagents for, for example, nucleic acid amplification or other enzymatic reactions such as primer extension reactions, hybridization, ligation, electrophoresis (e.g., capillary electrophoresis), mass spectrometry, and/or laser-induced fluorescent detection. The kit may also include instructions for using the kit.

Microfluidic devices may also be used for analyzing SNPs. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more of the disclosed SNPs. For genotyping SNPs, an exemplary microfluidic system may integrate, for example, nucleic acid amplification, primer extension, capillary electrophoresis, and a detection method such as laser induced fluorescence detection.

### D. Pharmacogenetics

Subjects identified as having late-onset AD or as having an increased risk of developing late-onset AD can be selected for treatment or prevention of one or more symptoms associated with late-onset AD. Subjects can be treated therapeutically or prophylactically. Thus, in one embodiment, treating can include directly affecting or curing, suppressing, inhibiting, preventing, reducing the risk of developing, reducing the severity of, reducing the number of, or delaying the onset of, symptoms associated with late-onset AD, or a combination thereof.

The disclosed genetic markers can be used for pharmacogenomic evaluation of a subject to determine which therapeutic or prophylactic strategy is most likely to be effective in the subject and to predict whether a subject is likely to experience toxic side effects from a particular treatment of therapeutic compound. Pharmacogenomics deals with the roles which clinically significant hereditary variations (e.g., SNPs and haplotypes) play in the response to drugs due to altered drug disposition and/or abnormal action in affected subjects (Roses, Nature, 405:857-865 (2000); Gould and Rothberg, Nature Biotechnology, 19:209-211 (2001); Eichelbaum, Clin. Exp. Pharmacol. Physiol., 23(10-11):983-985 (1996)). Pharmacogenomics as it relates to AD is discussed in Cacabelos, Ann. Med., 34(5):357-79 (2002); Maimone, et al., Eur. J. Pharmacol., 9:413(1):11-29 (2001); and Poirier, Mol. Diagn., 4(4):335-41 (1999)).

The clinical outcomes of these variations can result in severe toxicity of therapeutic drugs in certain individuals or therapeutic failure of drugs in certain individuals as a result of individual variation in metabolism. Thus, the SNP genotype or haplotype of an individual can determine the way a therapeutic compound acts on the body or the way the body metabolizes the compound. For example, SNPs in drug metabolizing enzymes can affect the activity of these enzymes, which in turn can affect both the intensity and duration of drug action, as well as drug metabolism and clearance.

The pharmacogenomic characterization of an individual permits the selection of effective compounds and effective dosages of such compounds for prophylactic or therapeutic uses based on the individual's SNP genotype or haplotype, thereby enhancing and optimizing the effectiveness of the therapy. Furthermore, the production of recombinant cells and transgenic animals containing particular SNPs/haplotypes allow effective clinical design and testing of treatment compounds and dosage regimens. For example, transgenic animals can be produced that differ only in specific SNP alleles in a gene that is orthologous to a human disease susceptibility gene.

Pharmacogenomic uses of the disclosed genetic markers provide several significant advantages for patient care. For example, pharmacogenomic characterization of an individual, based on an individual's SNP genotype or haplotype, can identify those subjects unlikely to respond to treatment with a particular medication and thereby allows physicians to avoid prescribing the ineffective medication to those subjects. On the other hand, pharmacogenomic characterization of an individual may enable physicians to select the appropriate medication and dosage regimen that will be most effective based on an individual's SNP genotype or haplotype. Furthermore, pharmacogenomics may identify patients predisposed to toxicity and adverse reactions to particular drugs or drug dosages.

### E. Identification of Additional Genetic Markers

The disclosed genetic markers are useful for identifying additional genetic markers associated with the development of late-onset AD. For example, the SNPs disclosed above can be used to identify additional SNPs that are in linkage disequilibrium. Indeed, any SNP in linkage disequilibrium with a first SNP associated with late-onset AD will be associated with late-onset AD. Once the association has been demonstrated between a given SNP and late-onset AD, the discovery of additional SNPs associated with late-onset AD can be of great interest in order to increase the density of SNPs in this particular region.

Methods for identifying additional SNPs and conducting linkage disequilibrium analysis are well known in the art. For example, identification of additional SNPs in linkage disequilibrium with the SNPs disclosed herein can involve the steps of: (a) amplifying a fragment from the genomic region comprising or surrounding a first SNP from a plurality of individuals; (b) identifying of second SNPs in the genomic region harboring or surrounding said first SNP; (c) conducting a linkage disequilibrium analysis between said first SNP and second SNPs; and (d) selecting said second SNPs as being in linkage disequilibrium with said first marker.

### F. Additional Diagnostic Methods to be Used in Combination

Detection of the disclosed genetic markers may be used in combination with one or more additional diagnostic approaches for identifying subjects as having late-onset AD or as having an increased risk for developing late-onset AD. For example, subjects can be screened for additional genetic markers in addition to the genetic markers disclosed herein. Subjects can also be subjected to a mental status exam, such as the Mini Mental State Exam (MMSE) to assess memory, concentration, and other cognitive skills. The subject an also be subjected to imaging procedures, such as a CT scan, an MRI, or a PET scan to identify changes in brain structure or size indicative of Alzheimer's disease.

### EXAMPLES

### Example 1

### Identification of Single-Nucleotide Polymorphisms (SNPs) Associated with Late-Onset Alzheimer's Disease in Two Extended Families

Materials and Methods

Family 1

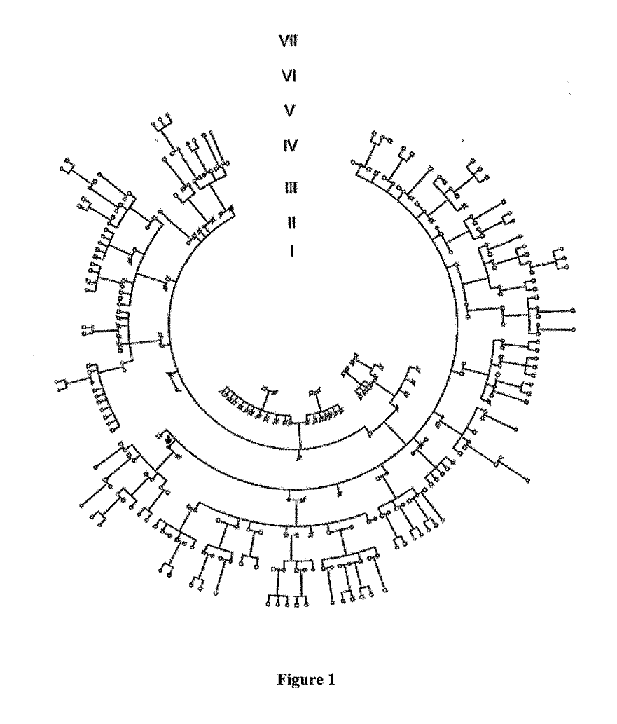

The proband was one of 15 siblings, 5 affected with Alzheimer's disease (FIG. 1). The patient developed memory loss at age 70. At age 77, the patient had a recorded Mini-Mental State Examination (MMSE) of 19/30, with an anomia for low frequency words and difficulty following serial commands. The magnetic resonance imaging (MRI) scan of the brain showed cerebral atrophy with several bright spots in the periventricular region, consistent with arteriosclerotic disease. Moderate prominence of the ventricles was noted. The electroencephalogram (EEG) was unremarkable. There was no history of alcohol or tobacco use. Blood work showed an elevated cholesterol (234 mg/dl), low density lipoprotein (LDL) (149), and a B12 deficiency which was treated. The patient's cognitive functions continued to worsen and the patient died recently at age 82 in a nursing facility. One sibling developed memory loss at age 72. At age 75, the MMSE was 20/30. Blood work, including thyroid function and B12, was unremarkable. The computed tomography (CT) scan of the brain revealed mild volume loss with no evidence of strokes, hemorrhage, or lesions. The patient has no history of alcohol use, but does use snuff. The patient is currently living with a child. A second sibling developed memory loss at age 70. The blood work was unremarkable with a normal electroencephalogram. The CT scan of the brain revealed generalized atrophy, but no acute abnormality. The patient has no history of alcohol use, but also uses snuff. The patient is living with a spouse. A third sibling developed memory loss at age 66. An MRI scan of the brain at age 67 revealed mild diffuse cerebral atrophy and small vessel disease. The blood work, including thyroid function and B12, showed a low B12 which was treated. The EEG was abnormal because of a mild slowing. This sibling never attended school but held a job as a telephone repair person. The patient had a history of drinking beer and using snuff. The patient died recently at age 74 in a nursing facility. A fourth sibling developed memory problems at age 65. At age 73, the patient scored a 21/30 on the MMSE. The blood work, including thyroid function was normal, but the sibling is being treated for hypothyroidism. The MRI scan of the brain at age 68 revealed a normal scan. There is no history of alcohol use, but the sibling does use snuff. The patient lives with a child. There are 7 siblings who currently are ages 60-73 with no signs of memory loss at this time. The proband's father died at age 58 of colon cancer and the mother at age 78 with signs of dementia. The proband's father's sibling developed memory loss around age 85 and died at age 93 in a nursing facility. A second sibling of the proband's father developed memory problems at age 89 and lives in a nursing facility. A third sibling of the proband's father also had dementia, but no medical records are available. A sibling of the mother is 84 and has no signs of memory loss. DNA was obtained from all of the siblings, the father's two affected siblings, the mother's unaffected sibling, most of the children and spouses for a total of 69 samples. All participants or the authorized representatives of the patients gave consent for the study, in accordance with the Institutional Review Board guidelines.

Family 2

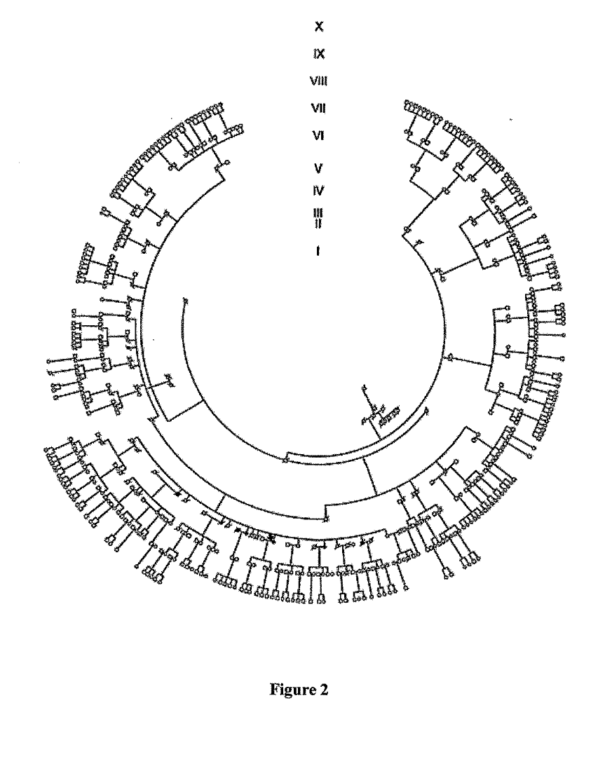

The proband was one of 14 siblings, 6 affected with Alzheimer's disease or dementia (FIG. 2). The patient developed memory loss at age 76. At age 77 the patient had an MMSE of 24/30. The blood work, including thyroid function and B12 levels, were unremarkable. The CT scan of the brain at age 80 years showed age related atrophy with prominent ventricles. There is no history of tobacco use and occasional to moderate alcohol use. The patient who was a school teacher and administrator lives at home with a spouse, as the cognitive functions continue to deteriorate. One sibling developed memory problems at age 80. The patient's blood work, including thyroid function, was unremarkable. There is possible alcohol abuse. The patient currently lives in a nursing facility with progressive worsening of cognitive functions. A second sibling developed memory problems at age 55. This sibling suffered head trauma and was unconscious for a time earlier in life. There was a hospital admission for paranoia at age 63. A CT scan of the brain at age 60 showed mild to moderate, deep and diffuse cortical atrophy. The blood work was unremarkable. Alcohol abuse was indicated. The patient continued the decline in cognitive functions and died at age 65. A third sibling developed memory loss at age 80. No medical records were available. There was no alcohol abuse. The patient died at age 84. The fourth sibling developed symptoms at age 69 and in addition to a decline in cognitive functions was aggressive and had hallucinations. The patient died at age 72. Medical records were unavailable. The fifth sibling is an identical twin to an unaffected sibling. The affected sibling developed symptoms at age 71 and is currently in a nursing home at age 82. A sixth sibling is currently showing signs of mild cognitive impairment. The proband's father died in an accident at age 58. He had 4 siblings, one of whom died in an insane asylum at age 60. The proband's mother died of leukemia at age 80. She had 5 siblings, one with possible dementia who died at age 88. There was no alcohol abuse in the parents. DNA was obtained from 5 siblings, 3 of those who were/are affected, the one with possible MCI, and from the children and spouses for a total of 71 samples. All participants or the authorized representatives of the patients gave consent for the study, in accordance with the Institutional Review Board guidelines.

Genotyping

Genomic DNA was extracted using the Qiagen Q1Aamp DNA blood midi kit (Qiagen, Inc., Valencia, Calif.) and suspended in low EDTA TE buffer. Aliquots were quantitated using the NanoDrop spectrophotometer (NanoDrop Technologies, Wilmington, Del.). For stage 1, five patients, 5 siblings, and 1 spouse from family 1 and four patients, 2 cousins, and 2 spouses from family 2 were used for the initial microarrays. Samples were diluted to 50 ng/ml and sent to Precision Biomarker Resources, Inc. (Evanston, Ill.) for genotyping, according to the manufacturer's specifications (Affymetrix, Santa Clara, Calif.), using the GeneChip® 500 K Mapping Array Set, consisting of two arrays (Nsp I, ˜262,000 SNPs and Sty I, ˜238,000 SNPs). Genotype calls were obtained from the Bayesian Robust Linear Model with Mahalanobis distance classifier genotype calling algorithm (BRLMM) on the Affymetrix platform. Among the 500,568 SNPs on the microarrays, 469,218 had call rates≧95% with HWE P>0.001, and were further analyzed. Gender calls were in accordance with the X chromosome genotype data and the known gender. Genotypic association was performed using the trial version of HelixTree software (Golden Helix, Bozeman, Mont.). Allelic and haplotype association were performed using the HaploView software (www.hapmap.org) (Barrett, et al., Bioinformatics, 21:263-265 (2005)). Bonferroni corrections were made using the 500,568 samples for multiple testing.

Results:

In stage 1, genotypic analysis of the microarray data involved analyzing the affected Alzheimer's patients' family samples against the control CEPH data with genotypic analysis, located on the Affymetrix website: (www.affymetrix.com/support/technical/sample_data/500k_hapmap_genoty pe_data.affx). The CEPH controls (60 samples) that were used were the unrelated parents. There were 6 SNPs on chromosome 2001.22 that were significant, after Bonferroni correction for 500,568 SNPs, with P values ranging from 1.23E-04 to 9.98E-05 (Table 1).

TABLE 1
SNPs associated with Alzheimer's patients versus CEPH controls

| | dbSNP RS | | Physical | | | | | FDR | |
|---|---|---|---|---|---|---|---|---|---|
| Probe Set ID | ID | Chip | Position | Chromosome | Cytoband | P | aP | (aP) | bP |
| SNP_A-2161805 | rs6087664 | Nsp | 33089877 | 20 | q11.22 | 5.63E−11 | 4.73E−10 | 6.65E−06 | 9.98E−05 |
| SNP_A-1793643 | rs6088692 | Sty | 33102249 | 20 | q11.22 | 5.63E−11 | 4.73E−10 | 3.21E−06 | 8.99E−05 |
| SNP_A-1961453 | rs6120816 | Nsp | 33108019 | 20 | q11.22 | 6.99E−11 | 5.84E−10 | 7.70E−06 | 1.23E−04 |
| SNP_A-2059637 | rs1885119 | Nsp | 33109310 | 20 | q11.22 | 5.63E−11 | 4.73E−10 | 6.65E−06 | 9.98E−05 |
| SNP_A-2208157 | rs2065108 | Sty | 33170483 | 20 | q11.22 | 3.85E−10 | 3.10E−09 | 1.68E−05 | 5.89E−04 |
| SNP_A-2153441 | rs6088727 | Sty | 33177300 | 20 | q11.22 | 3.85E−10 | 3.10E−09 | 1.68E−05 | 5.89E−04 |

Several SNPs on other chromosomes had significant P values after Bonferroni correction, but most were not in identified genes. All of the six significant SNPs on chromosome 20q11.22 were located in the gene for TRPC4AP.

The genotypes and frequencies of the four SNPs with the lowest P values for the Alzheimer's patients compared with the CEPH controls are listed in Table 2.

TABLE 2
Genotypes of the chromosome 20q11.22 SNPs from the
microarrays

| | A-2059637 | A-2161805 | A-1961453 | A-1793643 |
|---|---|---|---|---|
| Alzheimer's | BB (100%) | AA (100%) | AA (100%) | AA (100%) |
| CEPH | AA (38.3%) | AA (13.3%) | AA (13.6%) | AA (13.3%) |
| Controls | AB (48.3%) | AB (48.3%) | AB (49.2%) | AB (48.3%) |
| | BB (8.3%) | BB (38.3%) | BB (37.3%) | BB (38.3%) |

While all of the patients have homozygous genotypes for the SNPs, most of the controls have either heterozygous (50%) or opposite homozygous (37-38%) genotypes.

### Example 2

### Identification of Additional SNPs in TRPC4AP in the Two Extended Families and Haplotype Analysis

Materials and Methods:

Genotyping

For stage 2 of the project, additional SNPs in the gene, TRPC4AP, were selected from the NCBI SNP database (www.ncbi.nlm.nih.gov/snp). Three of the most significant SNPs from the microarray data and those seven selected from the database were genotyped in 69 samples from family 1 and 71 samples from family 2, using fluorescent-detected single base extension with the SNaPshot Multiplex kit (Applied Biosystems, Foster City, Calif.) as described (Table 3) (Huang and Poduslo, J. Med Genet, 43:e42 (2006)). Controls were the unaffected spouses in the families and our selection of 85 spouse controls.

TABLE 3
TRPC4AP SNPs analyzed in the extended pedigrees.

| | | Contig | | | | 1st PCR |
|---|---|---|---|---|---|---|
| | SNP | pos. | Chr. pos. | 1st PCR primer | | (bp) |
| rs1058003 | A/G | 3786509 | 33054078 | TRPC4AP-9F | CCACAGCCAGTTTCCTTCTC | 333 |
| | | | | | (SEQ ID NO: 2) | |
| | | | | TRPC4AP-9R | TTTCCTGTAGCTCCTCTGGTTC | |
| | | | | | (SEQ ID NO: 3) | |
| rs3746430 | C/T | 3789766 | 33057335 | TRPC4AP-17F | CTTGTGGGTAGGGAGTGAGG | 468 |
| | | | | | (SEQ ID NO: 4) | |
| | | | | TRPC4AP-17R | ACCAGGGAGCTGTTGATCTG | |
| | | | | | (SEQ ID NO: 5) | |
| rs3736802 | C/T | 3800134 | 33067703 | TRPC4AP-43F | GCAGATGCTGAATGCTTCCT | 272 |
| | | | | | (SEQ ID NO: 6) | |
| | | | | TRPC4AP-43R | GAAAGTGGGTTTGTGTGTGCT | |
| | | | | | (SEQ ID NO: 7) | |
| rs6088677 | C/T | 3803556 | 33071125 | TRPC4AP-52F | TTATGCCCTGGCTTTCTCAA | 268 |
| | | | | | (SEQ ID NO: 8) | |
| | | | | TRPC4AP-52R | TGTCCCTTCCTTCAATACGG | |
| | | | | | (SEQ ID NO: 9) | |
| rs6087660 | C/T | 3812620 | 33080189 | TRPC4AP-71F | CAGTTCTCTGTTAGTCCTTGTTGG | 357 |
| | | | | | (SEQ ID NO: 10) | |
| | | | | TRPC4AP-71R | CTGCCTTGGCCTCTCAAAGT | |
| | | | | | (SEQ ID NO: 11) | |
| rs4911460 | A/G | 3820422 | 33087991 | TRPC4AP-84F | AAAGAAGCCAAGAGCAGTGG | 556 |
| | | | | | (SEQ ID NO: 12) | |
| | | | | TRPC4AP-84R | GGACTACAGGCGCACGATAC | |
| | | | | | (SEQ ID NO: 13) | |
| rs6087664 | C/G | 3822308 | 33089877 | TRPC4AP-90F | AAAAGCAGAATGGTGGTTGC | 571 |
| | | | | | (SEQ ID NO: 14) | |
| | | | | TRPC4AP-90R | TTGACTCCTGTTTGTACAGATGGT | |
| | | | | | (SEQ ID NO: 15) | |
| rs13042358 | A/G | 3830571 | 33098140 | TRPC4AP-110F | AGTGAATGTGCCCAAAACGA | 214 |
| | | | | | (SEQ ID NO: 16) | |
| | | | | TRPC4AP-110R | CAGGGCTAGAGGAACTGGTG | |
| | | | | | (SEQ ID NO: 17) | |
| rs6120816 | C/G | 3840450 | 33108019 | TRPC4AP-130F | CACTGCTCTGCATGTCTCACT | 413 |
| | | | | | (SEQ ID NO: 18) | |
| | | | | TRPC4AP-130R | TTTGGTGAATGCCCTCCTAC | |
| | | | | | (SEQ ID NO: 19) | |
| rs1885119 | C/T | 3841741 | 33109310 | TRPC4AP-133F | GGTTGGAAAACAAGGACCAG | 508 |
| | | | | | (SEQ ID NO: 20) | |
| | | | | TRPC4AP-133R | CCCCAAACCTGTAGAAATCAG | |
| | | | | | (SEQ ID NO: 21) | |

Sequencing

Patients, unaffected spouses and unaffected siblings were used for the sequencing in search of the mutation causing the disease in these families. Primers for the exons and junction areas were selected for TRPC4AP from the Ensemble Probe database (www.ensemble.org/homo_sapiens/exonview?db=core&exon=&transcript=enst00000252015&flanking=50&sscn=25&fullseq=yes&submit=go) (Table 4). Samples were sequenced according to the VariantSEQr® protocol (Applied Biosystems) using the BigDye® Terminator Ready Reaction Mix v 3.1 and the ABI 377 sequencer. SeqScape® v 2.5 was used for analysis (Applied Biosystems).

TABLE 4
Primers for sequencing.

| Probe | Ex. | Forward Primer | Reverse Primer |
|---|---|---|---|
| Pr001109876.1 | 1 | tgtaaaacgacggccagtTCCAGCCTCGTACCTGCACC | caggaaacagctatgaccAGCAGGCAGGAAGCGGACTC |
| | | (SEQ ID NO: 22) | (SEQ ID NO: 23) |
| Pr001109890.1 | 2 | tgtaaaacgacggccagtAAAGAATGTCATCCCAATCA | caggaaacagctatgaccCCTTTGCCGTATCAGCTATT |
| | | CAGGA | ATCATCA |
| | | (SEQ ID NO: 24) | (SEQ ID NO: 25) |
| Pr001109928.1 | 3 | tgtaaaacgacggccagtTGCTtCAACATGTAAATGCC | caggaaacagctatgaccTTCATGGGAACTCCAGTTGG |
| | | GC | GA |
| | | (SEQ ID NO: 26) | (SEQ ID NO: 27) |
| Pr001109926.1 | 4 | tgtaaaacgacggccagtTCCCAAGGAAACATAGAAGC | caggaaacagetatgaccGGCCATGAATGGTTATGGCA |
| | | TGGA | A |
| | | (SEQ ID NO: 28) | (SEQ ID NO: 29) |
| Pr001119299.1 | 4 | tgtaaaacgacggccagtCAGCTGCTGGGAACTGCCAC | caggaaacagctatgaccGGGCAAGTAGGTGGCCAAGC |
| | | (SEQ ID NO: 30) | (SEQ ID NO: 31) |
| Pr001119292.1 | 5 | tgtaaaacgacggccagtGCTGCAGCAGTCCTGGCTCT | caggaaacagctatgaccGGGCTTGTAGGTTCTGATGG |
| | | T | GC |
| | | (SEQ ID NO: 32) | (SEQ ID NO: 33) |
| Pr001109925.1 | 6 | tgtaaaacgacggccagtCCCAACGGTGATGAGTGAGG | caggaaacagctatgaccTCCCACAGTTAAGCCATGCC |
| | | G | A |
| | | (SEQ ID NO: 34) | (SEQ ID NO: 35) |
| Pr001118124.1 | 7 | tgtaaaacgacggccagtAAGGGCCTCAGAGCTATAAT | caggaaacagctatgaccTGCGAAAGAGAGGCACATCC |
| | | CTCAAA | A |
| | | (SEQ ID NO: 36) | (SEQ ID NO: 37) |
| Pr001109875.1 | 8 | tgtaaaacgacggccagtAGCAAGAATGGTCCCAGGCG | caggaaacagctatgaccTCCCAGAAATTGACCTCTTG |
| | | | GC |
| | | (SEQ ID NO: 38) | (SEQ ID NO: 39) |
| Pr001118125.1 | 9 | tgtaaaacgacggccagtTCCCGOTTCTOGAATCAGCC | caggaaacagctatgaccTCTCTGCGCTCACCTGGCTC |
| | | (SEQ ID NO: 40) | (SEQ ID NO: 41) |
| Pr001109865.1 | 10 | tgtaaaacgacggccagtTCAAGTGATCTGCCCGCTCG | caggaaacagctatgaccTGGGTTTGTGTGTGCTGGGC |
| | | (SEQ ID NO: 42) | (SEQ ID NO: 43) |
| Pr001109923.1 | 11 | tgtaaaacgacggccagtGCCACGCTGICCACTCTTGC | caggaaacagctatgaccTGCTGTTGATCAGATGTGGA |
| | | | AGTGA |
| | | (SEQ ID NO: 44) | (SEQ ID NO: 45) |
| Pr001118127.1 | 12 | tgtaaaacgacggccagtGAATGCACGAGACAAGGCGG | caggaaacagctatgaccTCCACAGGAAGTGGGCAGGA |
| | | (SEQ ID NO: 46) | (SEQ ID NO: 47) |
| Pr001109919.1 | 13 | tgtaaaacgacggccagtAATTCATGGCAGGGCCCGTA | caggaaacagctatgaccCCCAGTTGGAGCAGGAAGGC |
| | | (SEQ ID NO: 48) | (SEQ ID NO: 49) |
| Pr001109918.1 | 14 | tgtaaaacgacggccagtGCCTGCACAGGCATTTGGAA | caggaaacagctatgaccCGTCCGCTTCCCACACACAT |
| | | (SEQ ID NO: 50) | (SEQ ID NO: 51) |
| Pr001109917.1 | 15 | tgtaaaacgacggccagtACAAGCCACCCATTCCCACC | caggaaacagctatgaccAGGCCTGGTCCTGTCCTTGG |
| | | (SEQ ID NO: 52) | (SEQ ID NO: 53) |
| Pr001109904.1 | 16 | tgtaaaacgacggccagtAAGCCAATTGCCCTGGAAGC | caggaaacagctatgaccCCTGGTGTGTTAGGCTCACC |
| | | | GA |
| | | (SEQ ID NO:54) | (SEQ ID NO: 55) |
| Pr001109916.1 | 17 | tgtaaaacgacggccagtCACTTGCGAGCCCTCCTTCC | caggaaacagctatgaccGGTGCTCCTGGGCACTCTGA |
| | | (SEQ ID NO: 56) | (SEQ ID NO: 57) |
| Pr001109915.1 | 18 | tgtaaaacgacggccagtAGCAGAGGGTGACTGCCGGT | caggaaacagctatgaccGGGAGAGCTCTGTGGGTGGC |
| | | (SEQ ID NO: 58) | (SEQ ID NO: 59) |
| Pr001109914.1 | 19 | tgtaaaacgacggccagtTGAAGGAAAGGTGGGCATGG | caggaaacagctatgaccTTCCACAACCTGCTGCGCTT |
| | | (SEQ ID NO: 60) | (SEQ ID NO: 61) |

Results:

For stage 2, 10 SNPs were analyzed in each member of both families. Haplotype analysis of the ten SNPs revealed a common haplotype for the affected siblings (Table 5 and Table 6) which consisted of (as read from the forward strand and not the reverse coding strand) rs1058003: rs3746430: rs3736802: rs6088677: rs6087660: rs4911460: rs6087664: rs13042358: rs6120816: rs1885119: G:T:T:C:T:G:C:G:G:T.

TABLE 5
Haplotype/genotype analysis of TRPC4AP in siblings
(sib.) and spouses (sps.) of Family 1.

| | | Sib | Sib | Sib | Sib | | | | | | | | | | | | | | | | |
|---|---|---|---|---|---|---|---|---|---|---|---|---|---|---|---|---|---|---|---|---|---|
| | Pr. | 1 | 2 | 3 | 4 | Sib | Sib | Sib | Sib | Sib | Sib | Sib | | | | | | | | | |
| SNP | AD | AD | AD | AD | AD | 5 | 6 | 7 | 8 | 9 | 10 | 11 | Sps | Sps | Sps | Sps | Sps | Sps | Sps | Sps | Sps |
| rs1058003 | G | G | G | G | G | AG | G | AG | G | AG | G | AG | A | AG | A | AG | A | A | G | A | AG |
| rs3746430 | T | T | T | T | T | CT | T | CT | T | CT | T | CT | C | CT | C | CT | C | C | CT | C | CT |
| rs3736802 | T | T | T | T | T | T | T | T | T | T | T | T | CT | T | C | T | T | C | CT | CT | CT |
| rs6088677 | C | C | C | C | C | C | C | C | C | C | C | C | CT | C | CT | C | CT | CT | CT | CT | CT |
| rs6087660 | T | T | T | T | T | CT | T | CT | T | CT | T | CT | C | CT | C | CT | C | C | T | C | CT |
| rs4911460 | G | G | G | G | G | A | G | AG | G | AG | G | AG | A | AG | A | AG | A | A | G | A | AG |
| rs6087664 | C | C | C | C | C | G | C | CG | C | CG | C | CG | G | CG | G | CG | G | G | C | G | CG |
| rs13042358 | G | G | G | G | G | G | G | G | G | G | G | G | AG | G | A | G | G | G | G | AG | AG |
| rs6120816 | G | G | G | G | G | CG | G | CG | G | CG | G | CG | C | CG | C | CG | C | C | G | C | CG |
| rs1885119 | T | T | T | T | T | CT | T | CT | T | CT | T | CT | C | CT | C | CT | C | C | T | C | CT |

TABLE 6
Haplotype/genotype analysis of TRPC4AP in siblings (sib.) and spouses (sps.) of Family 2.

| | Proband | Sib. 1 | Sib. 2 | Sib. 3 | Sib. 4 | | | | | | | |
|---|---|---|---|---|---|---|---|---|---|---|---|---|
| SNP | AD | AD | AD | AD | MCI | Sib. 5 | Sps. | Sps. | Sps. | Sps. | Sps. | Sps. |
| rs1058003 | G | G | G | G | G | G | AG | G | AG | G | A | A |
| rs3746430 | T | T | CT | T | T | T | C | C | CT | CT | C | C |
| rs3736802 | T | T | CT | T | T | T | C | C | T | CT | C | CT |
| rs6088677 | C | C | CT | C | C | C | CT | CT | C | CT | CT | CT |
| rs6087660 | T | T | T | T | T | T | CT | T | CT | T | C | C |
| rs4911460 | G | G | G | G | G | G | AG | G | AG | G | A | A |
| rs6087664 | C | C | C | C | C | C | CG | C | CG | C | G | G |
| rs13042358 | G | G | G | G | G | G | AG | G | G | G | A | AG |
| rs6120816 | G | G | G | G | G | G | CG | G | CG | G | C | C |
| rs1885119 | T | T | T | T | T | T | CT | T | CT | T | CT | C |

All 5 of the affected siblings in Family 1, and 4 of the five affected siblings in Family 2, for which we have DNA, have this haplotype. Moreover, in all of the affected siblings, the genotype is homozygous for these SNPs. Genotypes for the control samples are generally heterozygous. Unaffected sibling 5 in Family 2 and siblings 6, 8, and 10 in Family 1 also exhibit the haplotype of the affected siblings; they are younger in age and do not currently have any cognitive problems. Affected sibling 2 in Family 2 has the homozygous genotypes for the last six SNPs, suggesting recombination between the fourth and fifth SNPs.

Each of the 19 exons and their intron/exon boundaries were sequenced. Additional SNPs were identified, most of which were from the SNP website (http://www.ncbi.nlm.nih.gov/sites/entrez?itool¼gene_full_report&dbfrom¼gene&cmd¼link&linkname¼gene_snp&idsfromresult¼26133). Additional SNPs in linkage disequilibrium with the disease were found in and near exon 3 (rs1998233), exon 5 (rs4911463 and rs49114620), exon 6 (rs2281626), exon 11 (rs1885117 and rs1885116), exon 14 (rs2273636), exon 15 (rs6060151), exon 16 (rs4911169 and rs4911168 and rs3746431), and exon 17 (rs752449) (Table SIII). SNPs in the patient samples that were unchanged from the control samples were in and near exon 2 (rs11480829), exon 4 (rs7354623 and rs7354641) exon 6 (rs11907019 and rs17092225), exon 8 (rs6060169), exon 9 (rs11905247 and rs11478027), exon 11 (rs6088675 and rs4387881), exon 12 (rs6058157), exon 13 (rs6141525, rs12625215, rs6120789, rs6088673, rs6088674), exon 14 (rs13045538 and rs2273637), exon 15 (rs11696609, rs14329, rs11552600, rs6060152), exon 16 (rs17092212), exon 17 (rs752448) and exons 18 and 19 (rs17092208, rs11481073, rs11482185, and rs11552601). Several unidentified SNPs were found in exons 2 and 16, but were not significant for the disease. No mutations were found in the coding regions. The introns are currently being sequenced, as it has been shown that most introns and intergenic regions are also transcribed and may play regulatory roles (Gingeras, Genome Res., 17:682-690 (2007)). The sequencing of exon 9 revealed that the TRPC4AP is isoform 1 (exon 9 is shorter in isoform 2). Further studies revealed that DNA from all of the samples were isoform 1. The only sequencing variant found was a frameshift insertion in intron 18 which was also found in the controls.

The haplotype (G:T:T:C:T:G:C:G:G:T) for the disease extends from 33,054,747 to 33,120,760 bp in the gene. This haplotype is found in one block which contains all 19 exons.

### Example 3

### Haplotype Analysis of Unrelated Patients

Materials and Methods:

Subjects

199 patients and 85 spouses from community based samples were screened for the haplotype. Medical records were obtained on each patient and a clinical diagnosis was made according to NINCDS-ADRDA criteria (McKhann, et al., Neurology, 34:939-944 (1984)) which included a documented progressive decline in cognitive function and appropriate blood work to rule out other medical conditions, including thyroid and vitamin B12 deficiencies. In addition, results from a CT scan or MRI of the brain which indicated cortical atrophy, but no evidence of strokes or tumors were included in the diagnosis. The patients were Caucasian, of European descent. Spouses of patients and of siblings were of similar age, ethnic background, and similar environmental exposure who served as controls. All participants or the authorized representatives of the patients gave consents for the study, in accordance with the Institutional Review Board guidelines. The standard power for the association analysis is 0.88 (Ambrosius, et al., Am J. Hum Genet, 74:683-693 (2004)).

Results:

There were 199 patients with Alzheimer's disease (primarily Caucasian; 135 female and 64 male) and 85 control spouse subjects (Caucasian; 54 female and 31 males) used for the haplotype analysis. The age-of-onset for the patients was 71±8 years, with a range of 50-92 years. The reference age for the spouses was 60±17 years, with a range of 50-88 years. The clinical diagnosis of senile dementia of the Alzheimer's type was made according to NINCDS-ADRDA criteria (McKann, et al., Neurology, 34:939-944 (1998)). The medical records were carefully reviewed to verify the progressive cognitive decline and to document appropriate blood work to eliminate other medical conditions, including thyroid and B12 deficiencies. A computed tomography and/or magnetic resonance imaging scan of the brain, which showed cortical atrophy with no evidence of strokes or tumors was also included. The spouses were of similar ages, ethnic background, and environment, which controlled for unmeasured risk factors, as well as age and race. Participants or authorized representatives for the patients gave informed consent for the study, in accordance with the institutional review board guidelines.

Genomic DNA was extracted from blood samples using either proteinase K digestion and chloroform extraction or the Qiagen QIAamp DNA blood midi kits (Qiagen, Inc., Valencia, Calif.). SNPs were genotyped using fluorescent-detected single base extension with the SNaPshot Multiplex kit (Applied Biosystems, Foster City, Calif.), as described (Huang, et al., J. Med. Genet, 43:e42 (2006)). Nine of the 10 SNPs were genotyped in all of the samples. The SNP (rs6087664) was not easily multiplexed and not used. The nine SNPs in physical order were rs1058003, rs3746430, rs3736802, rs6088677, rs6087660, rs4911460, rs13042358, rs6120816, rs1885119. Haplotypes were determined for each individual by use of the expectation maximization algorithm (EM), implemented in Helix-Tree, of which we had the trial version. Haplotype data with EM probabilities greater than 0.88 were exported to SAS for logistic regression analysis to determine the risk associated with each haplotype. Each haplotype was further compared with the combination of the other haplotypes.

Five major haplotypes with frequencies higher than 5% were estimated for the data. The five haplotypes as read from the forward strand, starting from rs1058003 as listed were:

H1: GTTCTGGGT
H2: ACCTCAACC
H3: ACTCCAGCC
H4: ACCCCAACC
H5: GCCTTGGGT

There were 143 patients (36%) and 45 controls (26%) with the haplotype (Table 7). The H2-H5 haplotypes had lower frequencies in the samples.

TABLE 7
Haplotype associations

| | H1 | H2 | H3 | H4 | H5 | Others | Total |
|---|---|---|---|---|---|---|---|
| Patient | 143 | 105 | 55 | 33 | 18 | 44 | 398 |
| | (35.93%) | (26.38%) | (13.82%) | (8.29%) | (4.52%) | (11.06%) | |
| Cont. | 45 | 57 | 32 | 15 | 10 | 11 | 170 |
| | (26.47%) | (33.53%) | (18.82%) | (8.82%) | (5.88%) | (6.47%) | |

When the H1 haplotype was compared with the combination of the other haplotypes by chi-square analysis, the results were significant: P=0.0282 and the OR=1.56 (95% CI: 1.05-2.32). The standard power for the association analysis was 0.88 (Ambrosius, et al., Am. J. Hum. Genet, 74:683-693 (2004)). The data obtained from using logistic regression to account for age-of-onset, gender, and APOE4 status for each haplotype were not significant. For example, the significance for APOE4 carriers with the Hi haplotype was P=0.3520; OR=1.36 (95% CI: 0.71-2.62). The results for APOE4 non-carriers were P=0.1980; OR=1.45 (95% CI: 0.82-2.55). Thus risk associated with the H1 haplotype appears to be independent of APOE status as well as age-of-onset and gender.

### Example 4

### Latent Classification Analysis of Various Clinical Phyenotypes

The latent classification statistical model, the Grade of Membership (GoM, developed at the Center for Demographic Studies at Duke University) was used to investigate the various clinical phenotypes simultaneously without multiple comparisons (Corder, et al., Rejuvenat. Res. 9:89-93 (2006); Gold, et al., J. Gerontol, 45:S43-S51 (1990); Manton, et al., New York: John Wiley & Sons (1994); Randall, et al. Neurochem. Res., 34:23-28 (2009); Woodbury, et al., Methods Inf., Med., 21:210-220 (1982)). The data are represented by model-based groups which are defined by the frequencies of the responses for the variables. Individuals are not assigned to a group. They are assigned a membership score for each group. The internal variable used to define the pure types was the presence of the multilocus genotype. External variables were the clinical phenotypes, which may be encountered during the Alzheimer's disease process: behavior changes, hallucinations, problems with calculations or language, or depression. Age-of-onset was also included as an external variable. The clinical phenotypes were determined either from the initial form completed by the families upon entry into the study or upon examination of the medical records. The data for the multilocus genotypes and clinical phenotypes were analyzed simultaneously. Missing or limited information and small samples sizes can be used without specifying a particular model.

Using the latent classification analysis with the diplotype H1H1 as the internal variable and the data from the Alzheimer's patients, three groups were identified, as expected (Table 8). There was a distribution of 80 in Group I, 104 in Group II, and 148 in Group III.

TABLE 8
Alzheimer's disease clinical phenotype associated with TRPC4AP
haplotype.

| | | | | |
|---|---|---|---|---|
| Attributes | Frequency | I | II | III |

Diplotype

| | | | | |
|---|---|---|---|---|
| H1H1 | 15.06 | 8.14 | 0 | 100 |
| H1other | 43.98 | 84.88 | 0 | 0 |
| Other | 40.96 | 6.98 | 100 | 0 |

Behavior changes

| | | | | |
|---|---|---|---|---|
| Yes | 75.30 | 72.09 | 75.81 | 88.89 |
| No | 24.70 | 27.91 | 24.19 | 11.11 |

Hallucinations

| | | | | |
|---|---|---|---|---|
| Yes | 41.77 | 38.55 | 40.68 | 62.50 |
| No | 58.23 | 61.45 | 59.32 | 37.50 |

Calculation difficulty

| | | | | |
|---|---|---|---|---|
| Yes | 70.19 | 70.24 | 68.33 | 76.47 |
| No | 29.81 | 29.76 | 31.67 | 23.53 |

Language difficulty

| | | | | |
|---|---|---|---|---|
| Yes | 69.33 | 69.88 | 67.74 | 72.22 |
| No | 30.67 | 30.12 | 32.26 | 27.78 |

Depression

| | | | | |
|---|---|---|---|---|
| Yes | 54.09 | 51.81 | 55.00 | 62.50 |
| No | 45.91 | 48.19 | 45.00 | 37.50 |

Age-of-onset

| | | | | |
|---|---|---|---|---|
| 50-60 | 11.45 | 22.09 | 0.00 | 0.00 |
| 61-65 | 14.46 | 18.60 | 12.90 | 0.00 |
| 66-70 | 18.07 | 11.63 | 20.97 | 39.89 |
| 71-75 | 22.89 | 19.77 | 27.42 | 22.22 |
| 76-80 | 22.89 | 19.77 | 22.58 | 38.89 |
| >80 | 10.24 | 8.14 | 16.13 | 0.00 |

Group III had the H1H1 diplotype. Group I was heterozygous, while Group II did not have the H1 haplotype. There is an indication that the Group III patients may have more behavioral changes, as well as psychiatric issues, such as hallucinations. Interestingly, the Group III patients were late-onset, with age-of-onset ranging from 66 to 80 years. Groups I and II had ages-of-onset that were more widespread. When either APOE4 or gender was used as external variables, there was a wide distribution among all three groups, indicating again that the risk associated with this haplotype was independent of gender and APOE status.

1. A method for selecting a subject for treatment of one or more symptoms of late-onset Alzheimer's disease (AD) comprising detecting in the subject's DNA a genetic marker in the gene encoding TRPC4AP, wherein the presence of the genetic marker is indicative that the subject has late-onset AD, or has an increased risk of developing late-onset AD, and selecting the subject for treatment, wherein the genetic marker is one or more single nucleotide polymorphisms (SNPs) selected from the group consisting of rs1058003, rs3746430, rs3736802, rs6088677, rs6087660, rs4911460, rs6087664, rs13042358, rs6088692; rs6120816, rs1885119, rs2065108 and rs6088727, relative to SEQ ID NO:1.

2. The method of claim 1, wherein the genetic marker comprises one or more alleles selected from the group consisting of a ‘G’ allele at rs1058003; a ‘T’ allele at rs3746430; a ‘T’ allele at rs3736802; a ‘C’ allele at rs6088677; a ‘T’ allele at rs6087660; a ‘G’ allele at rs4911460; a ‘C’ allele at rs6087664; a ‘G’ allele at rs13042358; a ‘G’ allele at rs6120816 and a ‘T’ allele at rs1885119.

3. The method of claim 1, wherein the genetic marker comprises the haplotype: rs1058003_G: rs3746430_T: rs3736802_T: rs6088677_C: rs6087660_T: rs4911460_G: rs6087664_C: rs13042358_G: rs6120816_G: rs1885119_T.

4. The method of claim 1, further comprising determining if the genetic marker is heterozygous.

5. The method of claim 1, further comprising determining if the genetic marker is homozygous.

6. The method of claim 1, wherein the detecting is carried out by a process selected from the group consisting of direct sequencing, allele-specific probe hybridization, allele-specific primer extension, allele-specific amplification, sequencing, 5′ nuclease digestion, molecular beacon assay, oligonucleotide ligation assay, size analysis, and single-stranded conformation polymorphism.

7. The method of claim 1, further comprising subjecting the subject to one or more additional diagnostic tests for late-onset AD selected from the group consisting of screening for one or more additional genetic markers, administering a mental status exam, or subjecting the subject to imaging procedures.

8. A method for selecting a therapeutic or prophylactic strategy for treatment or prevention of one or more symptoms in a subject having, or at increased risk of developing, late-onset AD comprising detecting in the subject's DNA a genetic marker in the gene encoding TRPC4AP, and selecting a therapeutic or prophylactic strategy for treatment or prevention of one or more symptoms in the subject based on the presence or absence of the genetic marker, wherein the genetic marker is one or more SNP selected from the group consisting of rs1058003, rs3746430, rs3736802, rs6088677, rs6087660, rs4911460, rs6087664, rs13042358, rs6088692; rs6120816, rs1885119, rs2065108 and rs6088727, relative to SEQ ID NO:1.

9. The method of claim 8, wherein the genetic marker comprises one or more alleles selected from the group consisting of a ‘G’ allele at rs1058003; a ‘T’ allele at rs3746430; a ‘T’ allele at rs3736802; a ‘C’ allele at rs6088677; a ‘T’ allele at rs6087660; a ‘G’ allele at rs4911460; a ‘C’ allele at rs6087664; a ‘G’ allele at rs13042358; a ‘G’ allele at rs6120816 and a ‘T’ allele at rs1885119.

10. The method of claim 8, wherein the genetic marker comprises the haplotype: rs1058003_G: rs3746430_T: rs3736802_T: rs6088677_C: rs6087660_T: rs4911460_G: rs6087664_C: rs13042358_G: rs6120816_G: rs1885119_T.

11. The method of claim 9, further comprising determining if the genetic marker is heterozygous.

12. The method of claim 9, further comprising determining if the genetic marker is homozygous.

13. The method of claim 9, wherein the detecting is carried out by a process selected from the group consisting of direct sequencing, allele-specific probe hybridization, allele-specific primer extension, allele-specific amplification, sequencing, 5′ nuclease digestion, molecular beacon assay, oligonucleotide ligation assay, size analysis, and single-stranded conformation polymorphism.

14. A kit for carrying out the method of claim 1, comprising at least one oligonucleotide detection reagent, wherein the oligonucleotide detection reagent distinguishes between each of at least two different alleles at the one or more SNP.

15. The kit of claim 14, wherein the detecting is carried out by a process selected from the group consisting of direct sequencing, allele-specific probe hybridization, allele-specific primer extension, allele-specific amplification, sequencing, 5′ nuclease digestion, molecular beacon assay, oligonucleotide ligation assay, size analysis, and single-stranded conformation polymorphism.

16. The kit of claim 14, wherein the oligonucleotide detection reagents are immobilized to a substrate.

17. The kit of claim 16, wherein the oligonucleotide detection reagents are arranged in a grid-like pattern on an array.


SEQUENCE LISTING (61 sequences)

SEQ ID NO: 1 - DNA, 90412 bp, Homo sapiens
tcatttttaaaataaagttatttaatagtctccatttaattggtttatttgctgttaataaattggcaacacaaggaagggccccatgtccaggctcaggcaggagcggctgcctcaggcagaggcagggcaggcacagctgccccgtgccccaagggatggagggaaaggcccctcacttctgggagaacccccttggatgaacacagcggccaatgagcaaacagaaccagaggagctacaggaaaatgaggcagaggggcaggcttgggggaaggcagcattctctggtacccaccacccctgcccagggctcagaagcctttgcctgggtccaaaggccagggacaagggaggcttggctcacagatggggggtgggggtcacccatatcttcctccccactctgggactgactgacagccaagaaaccggcaggagctcacacagagctaatgctaaatgtcctcttacctctgggtggccctgggccccagggttctgaaggaaaggtgggcatggtaccctgtcctcattatggggactgaggctctccatgacctagggccctcagacccagggggaccaagggcttctaggacttccctcccaccaagcctgtacccaaagacctggggcaggcagagagcagcagggaggaacgggaacagggcagggcgtgtggcatcgtacccacgctcacccacactggcccagcagcctcccgaggcctggcccaaggtcactcctcagtgaagtccctgtctatgtccatgtagggctcctcaatgtagctaacgagagcagagggtgactgccggtccgggttcaacaggatggacactgtctccttccagtatgagaagctgatgcaggagctctgggcaaagagggaggggctggggggcgcctggaggacccaggtcaagcccagcaccgggacacccctctgagaggccctgggtccgcggcccacccatcacccccttgagggtggggcactcacgttctctaggcaggtgctgtccttgtccttgtgcaggtagtgctgctgccagaagcgcagcaggttgtggaagttgttgagcaggaagccggggtacttcttgctgtgctccatccgctgcagcagccgcaggtacaggggcagccgctctttccgtcgggccagcatcaggatcaccaggctggtgttgaggcagctgacgttctcctgcaaggccacaggcccacagggtcaggggctggtgggagccccagtggcccctggtgaacctgggtagccatcaggcagggaggagagtggccccaggcctcgggccacccacagagctctccctgcacagaggtgagataggactgggggaagggggcccccagcagccccagtttcctgactgagcaccttcctcttccccacgcccacctcccaccccacctgaggcaacaacacctgtccctgtccagacctgggctcaagcatcactccttccggaaggtccgtcccaggcagggctgggtgcgtgtgttggggcggttatcccgcttcagaagtgccattcagcactagcctgcgcgcctcggtggggaaagagttctgccccactcaccatgcactcccacctggtgcttcacctcagaacggaaccaaagaccacctctgagaggtgctaagatcaccctcactctagaaaagaggggcaggaggctcagagaagggaaggctgcatgactattcagtgagggagttgggcagcctcaggtccgattccaaagcccagcagggcagaaggtgtgagggacgtggacatgggggaggcggggagagcactggcctgcaagtcagcctgtgacctgagggcacttgcgagccctccttccttaggccctgtctgtacaatgggacactgattcctcctgagcacagtcctcagaagagctgggtctccagagacgcacttctggagtgggggacagaaacctgctgggcaccaggacaaaggaagtggtttccaatcgaccttccctgctgtgtgtctgccggggcctctcacctgggtcagcgtctgcacgtggatgatgttgatgaggcggaagaggaaggacatctgcgtgggcacctgggatatgtaggcgagcaggcggcattcagacagtacctcggcaactgtggagggaggcaggggtgcagcaggtcaggcttaggcccagtggctgggtcgcccaggcctttcgtggagccccgcttgggtcctgactctgaagctaggagctcatcatcaaaacagcttcaacgaaagctgactgtggcagaaagaaggacctccagctgtcctgctaccaggcaaagccatgttctcagagtgcccaggagcaccccacctcatcctccagaaccaagctcagggcagctctccctcagccctccccagactggctccagacccgtgccctccatatcacacacagaagagttccctccacaggaagatggatccatgacgctgcattaagaaaaacaacgcaaaagttccactccttctgcagaggtcctagcgcaactggttttccacaccagctctcaggtgagtccggcccagtgctgagaccacaaggcacaggttctgctccgtccactagtgtcggagcaactgcttcccgcacaactgggaggcagggcaataaccgaaggtaccaaaatctgtgtggctgttcccctgaaacagaccctctggggcagaaaaaggaagcaaacggctaaattaccaacaaccgggatacttcttgctatcaatgacagtgaccgtgacaaggaacaagtgcctgactttttgggggtgtgggggggtcacaaaccctcaggagaatctgctggatgccacaagccctctcctaggaaaaaaaggaaacgcgtgcgcatacacatggaagtgcgttagcaagtccaggagggttcagatgcctgctgcaccacagggcacctgcgatgccacgcacagcacactgaggacgagggggccaactcatcgtgccaggcaataccagcccagaagaggaaggtctttttctggttttctttgcatcttgctagcaacaatggattttcaccttattaaagtgttgaacaaggcagcaaagccaattgccctggaagccgccaccttgaggccggccacgtgggcatctggtgcagctccctcagtcattcttgtctccctgctggagacagggtgtctgatgccagcattcttaccctgcatgacttgccttgacagcctgcctttcatgtacctttcatatccacctggttttcaaatcggtccagggacagagtgacacagcgcaccagcatgttggagtccaccagggagctgttgatctgcttcaggaatacctggaactatacagaaaccaagctcacagcaggcagtgggctgctggccaggtatggccatggcccggggggatagtcactacaaggggcatcagcgacctctaccagccccactgcttcagataggaagacagaggctcagataagctgagggacctcccctcaccacccaggtactaagaggcactccccggaattcagcacagatccgacactctctccagtggttttacgctcaagggtgctggattcctttaatttttacttttaattttacttcagctagcctgagctgggtttctgtcacacacactcggtgagcctaacacaccaggcccagtccctccctacagcggctcccaccgtggcacccaccatgctgcgtcacggcaggcgcacaagccacccattcccaccctcactccctacccacaagcagccccggttttcatccctgcattcccagggtctagcacaaagccagacagagcaggttccaatgaatgtttgccaaagactgcccagactcccccgtcgtctctaacgtaaaacctgtgcctaaagcctggcagaccaggactccaggtgaacttctggacaacccagcacactccacagagccctgggttaactttacctttgcatcggtgttgatatatttattgaatctcttgaatgcatcaacgttgaacttcatcagctcccccaggaggtcaaagtaactctggagcacatcccttgacttacactcgctgtccacaatgcagtaaaggatgtgctgggtgaggagggacagggtgaaggtgtcaacgtggctgcccccaccagggcctggcatggagctcagaccatgcattggctttgagcaagcatcctggggatggaactggcaacactgtctagattcttgtctgaggatgctggtggctccaaggacaggaccaggcctgacagacctcaccaggaggtggagcagactgggaatgcagcacaatctctcccctgcccagggcgtacagctgaagcctgtgacctgctctgtggtcaccaagatggccggggtgctcagctcctaggtgaggggagctgtcatcaccaggtctaaaacacaggccactgcctaagcactgaggtcattcaagtaaatgaaggacagaatttaaaagcaaaggacacattttgaccccatggtttgtctttgtgtttatgaacgaaattgtttaaatagagtttacaccaaaatactgagttttcatcaagagaataaagccaatttactcctttataaagttttctacattttcttgaagttacattgatttggatcaattcttttccacatggattttataattcgtgggtcagtttcttgtccatatgaaaaaatctgaaagaccaattataactttggccattctgctctaagttttactttttctgaagaccattattcaataaaggaaaattaacttttagtacaaaatatcttcagggtcaaaatgccttgctgggtaaccaaactgtcaccctcctgcatggcctggccaccctctaaacctgtcaccccgtaagcacccatggggaacccacagttggaaacccagggcaaaatctaaggcagcctgcacaggcatttggaagtcacgagatctgctctgctcctgatgaactgtttggggctcaggacaggacacagcaacgcgtgggctggggggagacggaacccaagacactggctgctccagatcatacctccaagaggcctcgcttcagcaggaacatctggtctgcataggaggtggtccctcggaggaaactctccacagcccgagcttgccaaaacctgcgacccaaatactggctcaggtggctaaggctgccctcttccggcccatgttcagttctccaaggggttgggagatgtttgttgccttttcatgtactcaacatttactgaacatctaccaagcatcaggcttcatcctgggcctggggacacagcagtgcccccatcctcacagagagccagctcagtggagaagtcttctatctgggtctgtctggaaggggctgtgtcacagaaagggtaacatctgggatcgaagggctggctcctgcgagcccccgtgaaacatactctaggccttgccctttcacaagaatgggaggacgtgcaattgaggggccaggaatgtgtgtgggaagcggacgaacacatgcagacaggccactgggctcacagacgatgggctggcctagggaagtagtggttgatgctgaccgagggacccagagggcacagcgggtatccacgacagggagtttggacaaggcactgaagtgacaaaggcccatgtgggcatgaagcctcctgacagctcacgcccggccctgatggaactctacctgtccactgacttcaagactcatcttagatgtatctaaggacctcagagaaggtctccctgtattcacccagcccccagccctagcagcggaagaggctttcctttgctgctatagctcctgggccaaccctggctccattccttcccacactgcattgaaacagcacagctctagggaattcatggcagggcccgtatcctagttctctcccacccataaggcaccagcacagaacaggtgcttggtgaatgctcggtcactgtgggctgcagaagaagctctcacatacctgtcgagtcaaaactacacaagatggcagtcaaaccacctctccccaacagtaattgtgcttcaggaggcatggacgctggtgactaaaggaggtattcctaacctcctgagaaacacctggctctgcagccccgcagaatcggcaggtacttttccccagactcacctgaaagacgactctgctggctccttcttcatgacctgcagcagacgagttaataagcccctcttcccatcacacaccaaactcctgaaatagaagagagatattggtgacagatctgagaggctgacaggggccctggggatgggtcattctgctggcttaggcggtgaaaaaccatggggacatttccaagtctgcaggatgccttcctgctccaactgggtccagagccccggagacccctgcagccacagaattaaactgccaagtgcattcccaccacatctcaggccagctttccttcccaggccagaaacatatccagaatcagacatggcttagaactgccagaggagagctgtttataccaaaagtgtgttgaaacaggtcatgccagtctgccagatggggaaagactctgccaatagggtgaggcccagcgtactccgtgcctgaggttcacttccagctggccttgggcaagtgtccacaactccaagcctcagcttcccagtctatgaagcaaaggcaggagatgaggaaatgactctggccccgtcatgggggaaggaagacggccaggcacgcaggggtctgggcccttcaaacagagcagctctgctgtgatccactgcagacatcagggcaggactgggtacaattttatttgaatgaagagttctgctaccttagaaaaaaaagattgggaggccaggagaggtggttcacacctgtaatccctggactttgggaggctcacttgaggccaggagttcgagaccagcctgggcaacatagtgagaccccatctctacaaaaaaaaagtaaaaacactagctgggaatggtggcgcacacctgtagtcccagctactcgggcggctgaggcaggagaatcacttgagctcagaagtttgtggctgcagtgagctataacaccactgctctgcagcctgagagaaagagcaagaccttgtgtctaaaaaacaaaacaaaacaaacaaacaaaaaaggttgggaaatcatggctcttgttggataaagtccactgtattctgacgcctgtgagactagccccccactgcctggctccaggtgaggcaaacctaacccaggtaatgcaacctaacacttggcagcaggtaagaggcttctgtctgtcctgcttggctgcagatgccaccaggcaagaaaggagcacataagcaaaggggctgaatgcacgagacaaggcggtggggcagtgcggagcacacatgacttctggcaggtgacctctctgaggctcacctcatctgtcaagtgcaggtactgatggggctcccctcctagggctgctgtgaggctccaatgagaaaatgcaggtgacatgaatacaatgcctggcacacaggagccctcaggacactgtagctgcatgtgagagaggacacgcgtgcatacctggatgtgctcaccggtgagcccccggggaacgtgggcagccgggatgggctgagggaaaagctgcacccctgcactcacctgtcggtgttgaggacagcttccacctcagggatgttggccttgagagagatggcactgagttcattcagctcctggttgttgagtaacaagtacttgttcctgaaacaaaatgggttggaggaggtaaaggacaggaactggggctgctgggtcagcaccaggctagtgtagggagtctcctgcccacttcctgtggacagaatctgagaagccaccaggcactttcctctagcacctctcactccagccagcacctgcaggagggaactctgggctctcaggcagtggaatctctccctggccccacccccatccccgacccctgcctgccacattcatcgacaaggccccttccctcagcagctctgggcctttctctttgcctacatttccacttgttttctcaaaggcaccaacacctcaacaggtccaatcacactcaagcctggccctgctgcagcagtcccaccaagcaccacggtcacacatcacacagaggcctggttaactgtgacacttcccccgacttgccatccaggcctgtcactgctgggtcctgtgttcccaggacaccagctccctttactaaaaggtatcccacaggaactgagtcatcacacggtgccaaaagtgatcttcagaatcaggactctgatcctgtcactcccggtgaaaacacttccacagcttcccactgcatttagaacagacatttcctgacttaggaggaggttatgtcctgataaacccatcgtatttgaaaatgccgttaagttgaaaacatattgaggccgggcgcggtggttcacgtctgtaatcccagcactttggaaggtccaggcgggtggatcacgaggtcaggagttcgagaccagcctggccaacgtggcgaaaccccatctctactaaaaatataaaaaattagctgggcgtggtggcacacgcctataatcctagctactcaggaggctgaggtagaattgcttgaacccagaaggtggaggttgcagtgagccaagatcatgccactgcactccagcctgggcaatagagcaagattccgtcttgaaaaaaagaaaaagaaagaaaacacatttaatatatctaacccactgaacctcctagcttagcctagcctacctggaatgtgctcagaacactgacttcagcctacagttggccataatcactggcaacacggcaggcacagcacagagtactggttgtctaccttggtgatcgtgtagctggtcaccatttttcttgaaatacattctgagctgcagctgcagctcagtgcccagcattgcaagagtattgaactgtttatcatgagcccaggaaaagatcaaaattcaaaattctacttctgtaccattgtgaagttgaaaaattctaagtggaccatctgtatataaaatccaggcctgtgactgtgacccacaacaccctgtgactgtgacccacaacgtcctgtgcagcccagcccagagccaagcccacatcactcccatcagctggccacaatggtcttccctcgttcaggggcttctggctagaacacacaccctctcacatgcctgttcactcaattcactacaagctgacacttcaggtcttggctggcaagccacttctgcaggggagagttcctgagtgtcctctggccagtcctggaccctgtcaaatgtctctcacagaccccgttcctctcctcctgaataccatctccacttatcactaatgctcaacggtggaatccttggatgaacaactgtctctgttttcagactctgcccttcatgagagcaggggccatggctgtttgcattcatcctgatatcccacagagtactcagactgaagagtctgatgaaagccagagactcacttcctcagggaaaacagcacagttccataacatatgtggtgccatttttcagaaggttaacatgcttcctggagcccttctatggctttcagccggaaaacctctcacagaggatgggtgcatatgatggaggtgccctggggccacagctgtctccaggcatgttctgttagctcaaatgcaaggtttttaaaatatttaaaccaacatttaaaatcaggacctttcacataaatcttaatttctgcctttgcttggcagaaaaaatcagaagttatgcaacagtgggcccagtttcctgtgcacacgcagctggctggagctgagctcactgaaattcttgttcactaaggtccccacatcacctccccaatgccaaggtcaagggtcagctgctagtcacccccttcctgcactgttttccttacagcagagagtaattttccttgtacattctccatcgaaggttaaaaaataaatgagcgcagaatgtctttcaagaaaaatgggagcacacctccttgtggagggaagaatcctccagctgctcagtaagcagtcggctttgcctgtctgaagttcctgccggcccctgggaatagccacgctgtccactcttgctttagaggggcagtaggttgagaaatccattgattccaaaggtagtaaggggccaacgacttccatactcagttcctggggtggctatgagggttagaagaaagcttggggcagtgcctgccataaggaagtctggaaagacgggaggtccgactgcagtgtcacacctgctaacactgaggggacagtgggcttccaccagctccagggcctcctctcgagctgaggtcagggaccagacaaggagatcgaggccttccgaagacagcccaacaactctccttgcagggacactcttgtacttactcgtggtggtcactgaagctctgaagaagcctcaaaaactgtatcttcaaggtgatgtcctgaaacacatgcacagcctcaatgttaggaccacagccacaaggttccaaaataacacaagcagacactctggtgggagcctgcttctctaagcctctgtcctcatgtaccatgaggacttttctttagttagaaaaccatctgttcacttccacatctgatcaacagcagattaagctaatgtggcccccttcctgctaaaaacatgccatcacactcaataaaaaataaggcggggcgtggtggttacacctgtaatcacagcactttgggaggccgaggcgggcagatcgcttgaggtcaggagcttgagaccagcctgggcaacatggcaaaccctgtctctactaaaaatacaaaaattagccaggcatagttgctgcgcctgtaattctggctactcaggaggctgaggcaggagaatcacttgaacctgggaggcggaagttgcagtgagccgagatcgcaccactgactgcactccagcctgggtgatggagactctgtcccaaaaatccaaccaaccaacaaacaaaacaacacgtgtggggaggagcgggggaagtccataggagttagaaatgaagaaaggaccgaggagtttggaatagtgagccaaggaactgatactcagcagctgtgggcagtgtggagaggtggagacaagacctagtcacactaaggcttgggttttaatgccctgcagggactagacagggtgtcaggtcccagtaaagcctggggagcatgtacacttggtgaaagaagggttagaaaggcctgacccaccagcccagggagcctggctctgccatggtgctgctggtgggtgaagggggccctcccaagagcagcagaaaccctgggctcatacgactggggcagtagggccaagacattaaataagaaaactggccctgggacagctgccagacaaaagcaaaagtgccctgtagaaatgcctccagggctgaggctacacaggtatccaagggggaaactaaccccactgacctcaagtgatctgcccgccttggcctctcaaagtgctgggattacgggtgtgagccaccatgcccgggagcacgtttctataggtggatgaaaattacaataggataaaacagcaaataaggaaaagcaggctgctagctcatgcagtgggcagccccctccctgcctggagtgaaatgagatccccatcacagagattacaaggggaagtggctagagggcctcaggcagaatttttttttttttttttttttgagacacagtctcactctgttgcccaggctggagtgcagtggcgtgatctcagctcactgcaacctctgcctcctgggttcaagcaattctggtggccgctccacagacattacaaaacacgaggcaacactccaccctgaggtctgaagacccaaacacaagcattagtgccccatgagcaatcgaaagagaccttaagaaagtattttaaaaatgattaaaaacagaatatgcagcagaaaaaagatattaacatcatggctaagcagtttccgaattgagccagacagaacatctagaaatgcaaaatatatttgcttgaaattaaaaactcaaatgataaagcagaagacaggtcgctgagggaagtccccagagtgcagtgaagagaagagggccccatggcaccaggccgatggctgggtggcacttttaggtcccagagcactttccttctcatgctggtaggcagggtgccgactagccggggggccaataggggaaagagtattatccttcttttgcaagaaaacagaggctcagagattcagtgattcattcaagctcttccaacaagccttccaatgtcctgttcagagctctctcaaggggactgtgagagacacaagctaaaggagagtgcttgggctcaaaaaaccaaagccacctttttttgtatgagacagtgtctttgctctgtcacccaggccggatgtgcagtggcttgatcttagttcactgcaacctctacctcccgggttcaagtgattctcccacctcagcctcctgagtagcagggattacaggtgccccaccaacatgcctggctaatttttgtatttttagtagagacggggtttcaccatgttggccaggctggtctcgaactcctgacctcaagtgatctgcccgccttggcctttcaaactgctgggattacgggtgtgaccaccacgcccgggagcacggttctataggtggatgaaaattacaataggataaaacagcaaataaggaaaagcaggctgctagctcatgcagtgggcagcccctgcctggagtgaaatgagatccctatcacagagattacaaggggaagttggctagagggcctcaggcaggaactttttttttttttttttttgagacacagtctcactttgttacgcaggctggagtccagtggcgtgatctcagctcactgcaacctctgcctcctgggttcaagcaattctggtgcctcagcctcccgaagagttgggattacaggcacccaacaccatacctggccaatttttgtatttttagtgagagacgcaagtgtctctcacactagtgagacagggtttcaccatgttggccaggctggtctgtaactcttgacctcaagtgatctgcccgctcggcctcccaaagtgctgggattacaggcctgagtcactgtgcccagcctaggaagatcttaaaagggatttctgcataggttggggtggggagtggagttcactcagatcctactccaaggtagcttccaattaggggactataagatatgattttagtttctgctctactcccaaaggaaaaaaggtcaaagggtcagcagcaggtcttgaaacaactactccaagccagtgaggccccatctgtccccgggggtctcctgcttaccgggctacagtcacagttctggttgtgaccatggaggacaagggcagatgctgaatgcttcctccaaatcagtttgtcaaacaaattattaagtccagggatcagcttgaactctgcaatcattctgtgaacctgaatgggataggaaggaaaaagtattaagacaaatgtgcttgaaaaatctagcaaaacctcgtgctcagtacacacgataatgatattcaggacaagtatcagtactgtcagtaaggagggccagggtctgaagaaaacatccgtgtatcccctatgcccagcacacacaaacccactttctaactttgctcattaatgctcacaataatcctacaaggcataatgtccggtagttcctatcacagaagcatgagtttgggattcagagagtcctgcatatgattcaagagtcctttgggatgcaagagtcctgggtttgactcttagctttaccatgtgcaaactttgtaaccttaaacaaattccttaagatttgtctcctcgtattaacatcaaaatgttcctcatgctttgcactaggataaatttcagctgaaagtaagatacaattattaaaaaactctaagatctagaaaaatctctgcaaagaaaaaaaaatgatctaagagttggaaaagcctttctgaaggattataataaacctagaagctcaaaaagaagcttgttgacatcaaccacctaaacctttaaaaattctgtatggcaaaactcaagcaaagtcacaagccaaatatgacaaacagagaacatacattggcaattcatgccacaggcaaagaaattcctctataaggagtcctacaaatgtctctgatttttcactttttcagactgggaaagatgacaaagctttataagaccgtgttggtgacttgggcattgggtacattcacacgctgttggtgggagggcacactggtatgacctgcacagggaagagtgtttggcagtagctctaaaattgcaaatccataatccctttaaaccagcaattccactttaggaatttacactcagacagatccatataaacactaaatgatatatatttacaacactactcactatagcattatttgtaataccccaaaataaattatggtatgccactcaatgggatacaaggcaccagtaaaaaagaatcaaggtattttatatgtcgtgaactgaacaattgccgagagataatactgggaaagcaagtgtgctcatgctgtattactgattgtgtaaaaaaagaggaatacaattcgcctttgtaagcataaaaatgtctctagaaggatacactaataactggtaaaattagctgcttcatggaaccattagaatagaatgtcaaggatcagaggggagagaggcttcacttatactaatttatatcttttaaaacagaactgcaggaatgatttccctgttaaaatttgtttatataacataaggctattactagcccttctcggggctgttgtaaggatcgagtcagaaagcagatgtaaagctctgcacacagcaccagtgctcgcttattgctattttacaagggggagaagttcagcaagaccagcctgggtaaggtcagtgattgcaacggcacctaactcaggtgttcagatcttaagtctgcccctttctagcccactacaggtatgcatttatcttgggagaccagaagctgacaaagacatccctggcagcctccatttccatggacctcttccttcaacatattaggttgtgtaaaagtaattgcgatttttgccactgaaagtaatagaggtgactattctcttcctactagaaatcacagcctctggccagtgttaagactcataagcagcccagaatctgttagactgaggctgatgtaaaatgacacctgttctgccactcaggcctttactaaaacccagcctggatctaccttgccctgcttaactgggggtggggaggtacggctgacatctatggtgttcaccaagccatgtgccttcctgtctcactccaaagggccctaagccaggcccaggtcacacagctataaaatgttccaacaatggcccaaaaaaattcatccagtgcaattgcctcactgcacagactagaaacccaagcttaagagattaggggtgagtcacacagctgggaagagaggggccttctaaggcagacttggacctgaccctgggtctcctaagttcaggccagtgcttaggtttgaggtttctactacatcatttcccttttctcttaacaataccagcttctcggccgggcatggtggctcacgcctgttaatcccagcactttgggaggccaaggcgggcggatcacttgaggccaggagtttcagaccatcctggccaacatgatgaaaccctgtctctactaaaaatacaaaaattagctgggtgtggtggtgtgtgcctataaccccagctacttggagggctgaggcaagaggatcgcttgagctggggaggcagaggttgcagtgagccgaggtcatgtcactgcaccactccagcctgggtggcagagcaagactgtctcaaaaaaagaaaaaaaaaaaaaaaaaaaaaagaaagaaagaaaatagcagcttcttcatgtgtaaaatttgtaaaacaaattttacttgtagaatgcattattcctactctcaggtccagccccgtagcctgctctatagcaggatggtaggcaggtgggcgagatgaagtgccactctaccagaggaggcagcgggtacaggctcaggcatttatctctgcagtcagacagccctgggttcacaagatagctctgtgaccctgggcaacattctttgaacccaagtcccttgatagaaaaggcgatgggggacgatggagctgttgaaaaggctcagtgaggtactgtagaagctactcagggggcactaaaagaaacgactgctatatggatggttttcattagcaccagagggatgaaacttgacagcacatggggagcctttccagtcctgagatttcaatttggccaagattggtctgagaacttggcccaactgctcattaacgaggaggcctctttaaggaggaagccttctgtgaggcctggccaaggaaacccttccccttgctacccagtgtaggcaggatctgggaatccagatcctcacggtgcccggcacacagcagaaacccaggaaatgctgactgtgcaaatgagaacacgctagtgaaacagtggttttggggggccaggatttcaaggtcattagtggtcccttcccaaggaaccagcactgtaatcatacagtttaactcaagaccgcaacaactcatagcaacctggcaacaagaaggctcagggaacagagaactgctgtgagagggcagaaagaggagacagggcagcagagtttaaatcctggctctatccaggcctgtcccctgagcgcacagcccagtttatgccctggctttctcaagaatcgagtgtggggacacagagtcaccacctgttgtgtaacttactaaatacctgaaaattcaaacaactgtgaaagtgcctcgaaaaagtaatagcactcaatgcacagaagctgtccctacaattatttcacatataagcaactgcgaaagtgcctcaaaaaagtaatagcactcaatgcataaaagctgtccctaaaactattattttttattaaaaacatgttccgtattgaaggaagggacaccaatgctgtggctatcaccgtgggatctcatccagtgtcctggttttaaatacaaccatatgctgaggactcccaaagtgatacctccagccttgccatctccccaacaccaggtgtaggtaatcaatccaagtgtcaattctaacacacacctcaaatctaccttgtacaaaacacaactccccatcttcctccctaagcctgctcctccaccccttacctgctagtcttaaccacctcaatacctggcaaatccattctcctacttccttgggcgaaaaaccttgggagtcacccttaactcctctcctcactcttaagttcacatccaaaccatcagcaaatcctatctttcaaaacatatccagaatgcaaccacctcccaccagtcccaccatcaatgtgctggtcctagctgctatcatctcttggccctattatgagggcccctaatggtcgccctattcccttccttgctgtttccagtctttccaaaagcagtcagaggaatgacttctcacttcactcagagtaaaaaccaaagtctttaaaatggcctgtgacagcttgacatgacctggtccctgtgagccctcagccctcctctcctggctgtttgctccaggctgtcccgcgctgcctcactgttccagcaactatgcacactcctccccagggcactgccccctcccacctggtacactctttcattctcttctggacgttttcatcctcacttgtaaaatgggggtaactgcacctctgttgcccagacttgtgacagtccagtgtctgactcagaaagatgtggaaggactgctgagcctccagcctctcctgcccccatgcttggtttcctccctagaagggcgcggtctgcaggaaaaggctacttctcaatagtcttgaggagggccgcactgttccaagccctttttcctccactgtaggttatgggcccctcctcctccgtcttggaactacttcaaggctcagggaaccttgcacatctggagctccctgtacttgcccagttttcctaagcagacacagctctgggctctgtcccactgccctgagcccacatcaggttggcaactcctctagcatgtccacagggccaggcttgccagctttcaactctttagtcttcacctcacagaggcaactgcctgcttctgccctcagcaccaaccaagactgcctagaaacgctccatacagcaaggcacagtgaaaagactcccggttctggaatcagcctggtttccttataggtagaacagaaggagaaatacccctatgctttgccaggctaaagcgagagccattctaacagaaggcccagtggagcacctggcataccatgagcacccgggcagcacctgctccccgtgtcctgagacgctggagaggggcccacctggtttcgctgacgccccatcagcagcacgcagaggacatagagcacttccagtttgtacatgatctcatgcataatcttcattgactggggcagctgggttctggctgacgtgtgaggcaggccattctcctcagaagcccctggaggagggaacacaatggaggctgacacagccaccggagacaggattgagtcacagcttgtgaggaaacacgtttctgcccctcgaacctgctctgaacagacttgtagcatggccattcggctgggccatgagcccaaaacacagactctgaaagctgcaaaactccctccacaatcccaggagccaggtgagcgcagagactcggaaaaatggactctgcaatagttgtgcctcatcaactttaacataaagtaaaaatattcggacacagccaatacagtaacatgacccatccctccctgcttatgcccttctcctgccaccaaccagacccccaggaagtgcccaggcagatgtatgacagcaaattatggggaccgctcttcaagtcgcaaatggccctgcttacttggttcttctgctgagactccatgagggacactgctgaagcccaagagggattctttttgaagaattccccgttttttaaaagaagaaccatctaaatttttctaaggttcaactttctaaagcagactgggtatcacaactgatgctactgatgcagatgaacatattatctacaggcagattttcattttacccaatgtcattactgctaacatcaacagcagctaagtgatatttatgggacacttgatatgtgtcagacttgctggctttatgactttgggccagtcacctcctcactaggccttagactgctctgcagcagacagctagggttactgtgcagactgaatgggaacgtaagagaagcccagcatctgattcttaggaagcccccattcactcctagatcctttctttacttatttacagagtcttgctctgtcacccaggctggagtgcagtggcatgaccttggctcactgcaacttccgcttgctggtttcaagcaagtctcctgcctcagcctcccgagtagctgggactacaggcatgtgccaccacgcccagctaatttttctatttttagtagacatggggtttcaccatgttggccgggctggtcttgaactcctgacctcaagtgatctgcccgcctcagcctcccaaagtgctgggattacaggcgtgagccacggtgcccagcgtagatccctactattttaagagggcattggtcagagtcaggagaggctaggtgatcttagcagagtgacttcccttcagtttcctcattttaaaggtgaacagatgaatcagtaactgccaaggtcctctcaagttcctacaggaactgcttccctagaggccttttgcaaaggcctctatgtgttatagaggagtggttatcagatatattcaggtaaggctcatacagaaaacacagtgatactctttaaagtatcaagacagaagaccctaacttattccgactaggtgtggtggctcatgcctatgatcccaggactttgggaggccgaggcaggtggactgcttgagcctaggagtttgagaccagcctggacaatacagtgagacctcattgctataaaaaaatttaaaaaattagctgggcatggtggcatgcgcctgtagtcccagctactcaagagggagaggtggaaggatcgcttgagcctggaaggtgaaggttgcagtgaaccaacagagatcgtgccactgcactccagcctgggcgacagagctggactgtctcaaaaaaaaaaaaaaaagagagaatctaacttactctatcctacagacttggaactatctttccccgtcatgttaaaatcacaaccttggaagacgacaagctgctgaatgaagtgtgactgagacacagtgtgacacggtagttaagagcactgaccctgaagccagactactgggtttgaaccccattcactagccgggtggtcttcagtaaatcagtggacctctctgtgcttcagtatccatcctcctttgtaaaacagcaatactgacaaccatacctaccccttaatgttgtaaggagtagagggatatactgaaggtgcctggaacatagccccaagtaagcaacatacaattagaaactaaatagaagttaagttctcagaaaactgagtttttctgcgaatgcttgcttgttactttgtattctttttcatctccaagcttttaatgggaactaaacaggcaggaggcaaagacttaggaaaaatttactggagaaatttttaatttaagagattctatctactcatatttattctcctgtaccttgggcaaaaaactatagttactcaatttccatgttagcactccttggcccagcttccagaacagttaagtgtcagctaatctgtgaagtgaacctgaagaactatcacgcagaatcctgcctctgtcgtgcactgctgtgagaccttgcgtgcttggcatcctcggcacccagcccatggtgctctagggagtcctgcagggaaggatgtttatgcaagcagtgggcacagtgtggggcacacaagtcctactgagaaatgaacgtctgcgaagaaagagacagggcagcctgacccctctccaagtcaccaccaacttttggtgcctggcttgcgcaggcctgacaagactttctgtgataaatattttcaagaaacagtctccaaagcaatggccagagcttagcaacaaggtcagtcccaaggatgaggatgggaaaaccagctcacgaggcctgtgctcccctgagtctcttcagctctaccacaccgattggagcggtcaccactctctgtctgcactgcactgttttctgcggacatgcacactcacttttttttttttttaacagctttgaggtacaattcacataccatgcaattcacccatttagttaaccatggtatcgtgatttttaggacattcatagagttgtgcagccattaccacaatcaattttagaatatcttcatcaccccccaaaagcagccttgtactcactaccagtcacttcctatttcccaccaaactccttagctctaggcaaccatgaaactattttccatctctatgcaattgcctgttcaggacacttcatattaatgaactcatgcaatatgccatcttttgtgactgcttctttcacctagtacaatgttttcaaggtttatccatgttgtagcccctatcagcattcccttttattgctgaataatatccgctagtataaatatatcacattctggcttggtacagtggctcaggcctgtaattccggcactttggaagaccaagacgggaagatcatttgagtgcaggagtttgagaccagcttgggcaacacagcaagatcctgtctctttttattacaaaaaataaaaaaattagctgggcgtggtggtgtgcacctgtttccagctactcgggaggctgaggtagaaggatggctgagcctgggaggtcagggctgcaatgagctgtgatcacaccactgcactgtagcctaggtgacagagagagaccgtctcaaaaaaaaaaaaaaaaaaaaaaaaaaaaaattctgtttattcattaatcaggtgacagtcatttgcgttgtttccatattttggctgttatgaatatgctgtgatgaacatacctgaacaaatttttgtgtaacatatattttcatttctcttgggtatatacctgggagtaggatttctaggccctatgacataactgtatgttaaccctttgaggaactgctagactcttttctaaagtggctgcactattctacatgcccactaatagtgactgagggctctgatttctccatattcttgccaacccttattatccaccttttttgatgataaccattctagtgagtatgaagtactatatcattgtggggtttgttttttcctctatcatctagactacttaagtgcagtggcaggatcacagctcactgcagcattgaactctggacccaaacaatcctcccacctcagcctcccaagttgtcaggactatgggcacgtgccaccatgcctggctaaccgaaacaaaatttttttttgtagagatgaggtcttgctctgttgtttgagctggtcttgaacaatcctcctgccttggcctcccaaagtattgggattacaggtgccagccactgtgcctggcctcactgtggttttgatttgcatttccctgacggctaatgatggtgaacatttttcacgtgcttaataattatttatatatcttctttggagaaacatctattcagatcctttgtctatattttgaaatgggtttttttattattgttatgagagttctttatacattctagatacaaatcccttattagatatattatttgcaaagatgtcccaccctgccttacccacaaaaaacgttcttccattctgtgggttctcttttcactttcctcattgtgttttctgaagaacaaaaagttttaattttgatgaagtccaatttattatttatctggttgcttgtgcttcagatgtcattgtgtatgacagctaaattgttaaatccaaggtcatgaagctttatccttatgttttcttctaagggtttcacaggtttagctcttacatttaggtctttgacccttttaattttgtatatgacataaggcaggggatctaacttcattctttcgcatgtggacatcaaatggtcccaataccatttgttgaaaagactactactgtttccaccaatgaatggtcttggtacccactgaaaatcaattgatcataataagcatgagggtttatttctgaactctcaacaatattccattgatcaatatgtctatcctcaggccagcagcatagtgtctcgattgcagctttgtagtaagttttcaagtcaggatgtgagttgtcttaggttgttttttttttttgagacagagtcttgctgtcacccaggctggagtgcagtggcacaatcacaggttactgcagcctcaaactcctgggctcaagctaccctcccacctcagcctcttgagtagctgggactataggtgggtgtcacgaggcttgcctaatttttaaattttttatagagacaggggtctcactatgttacccaggctggcctcaaactcctgaggccaagtgatcttcctgcctcagcctcccaaagtgctgggattacaggcatgagtcactgtacccggcctagttcaagactgtttttgttaatccaagtctcctgcatttccacatgaactttaggatcagctactccatttgtgcaaagtagtcagctgagagttatgacagaaattgtgttgaatctgtaaggctgatctatagatcagaatgggactatcattatcttaacaatattaattttctgattcaaagacatgggatgtctttacatttatttagggtttaatttctttcaatgatgttttagaattctcagagtataagttttacacttaagttcctaagtattttatttttttgatcctattataaataaaattgttttcttaatttttggtttgttgattgctactctatagaaatactactcattttttaatattagccttgtattctgaacctttgttgaacctatttattagtttactggtttttatagtagattccttagatttttctacatataagatcatcccatctgtgaacagtttcactacttttctaatttgcatgcctttaatttctttttcttgcctaactgccctagccagaacctccaatacgatgctgaagagaagtggcaatggcactgccacttccagaaacgatcaggaactcgtcttgttcctgatcttagtggtagaagctttcaattttttgccattaggtatgtcagccatgggtttttgtagatgccctttaacaggctgaggaagttctctcctgagtttttgtttttaccatgaaagggtgttggattttgtcaaatgcttttttgctcctatggaaacgatcatgtggtttttgtcctttattctattaacacagtgtattacataaactgattttcaaatgttgaaccaaacttgcataaatccatttggtcatggtatacaatccttgtatgttgctatacttggtgtgctagtattttgttgaagatttttgcacctataatcataagagatactggtcttgtgatgtctttatctggttttggtatcatgataatatgacctcaaagagtaagttgggaactgttccttcctcttttattttctattagagtctggaaagtattggtgtgaattcttcttgaaacatttggtagaattaaccagtgaagccatgtgggccaggacttttctttgtgggcagtttttcattatttattcaatctcgttgttatacttcaggttttctatttcttcctgagttagctgcaatagtttgtgtcttttaacttgctcatttcatctaggttatttagtttattggcttatattgtttatagtattcccttagagtcctcttactttcttaaggttggttgtaacgtaccttctttcatttctaatttagtaatttgagcctttcttttttggtcagtgtggctaaaagtttgtcaactttgttgatctttttaaagattacctttaaaaaaatcacctttggttttacagattttctctactgtttttctattctctatctcaccagttttcactctaatcctattacttccttttccttttgagatgtttgttctttttccaatgacttaaggtagacgtttaggcaatttagatttatttaaaaatgtaggcatttatagctctaaagttccctctaagcacgctttagctgcatcccataagtttgatatatagtatcattttcatttgtctcaaaatatttccttttttttttttgatggaatctcactctgtcacccaggctgtagtgtgatctcggctcactgcaacctctgcctcgggggttcaagcaattttcctgcctggacctcctgaccttaagcgacccgcctcctcggcctcccaaagggctgggactacaggtgtgagccaccgcgcccggcctcaagacacttcctaattttgtttgtgatctcctgtttgacccactggttatttagaagtatactgcttaatttgcatgtatttattaatttcccaagtttgttatttatttctaactcaccccactgggatttgaggacatacttttgtacagttttaatccttttaaatttgctgaacttttttttaacagcctagcatatggtctgtcctggagaatgttcatgtgcacttgagaagaacgtgcattctgtagtagttgggtggagggttctacagttctctgttagtccttgttggtatacagtgttatttaagtcttctgtttccctgttgatcttctgcttagttttatccattattaaaagtagtatgttaaggtctccagctgttgtttttgaactactgctctcttccaatctgttagtttttgcttcatgtattttttgggggctgtttttggatgcatacatgtttataactgttgtatcttcttgatggtttcaccctgttattatcaaatatccctattttgctctgataacagtttttgttttaaagtctctccagcttttttggctgggtggtggtggctcatgcctgtaatcccagcactttgagaggccaaggcaggcagatcacctgaggccatctgagaccaacctggccaacatgctgaaaccccatctctactaaaaacacacacaaaaatgagctgggcgtgggctccccgcctgtaatcccaactacttgggaggctgaggcatcagaattgcttgaacccgggaggtggaggttgcagtgagctgatcgtgccactgcactccagcctgggtgacagagtgagactctgtccctccaactttttaaaagtacagcctctccagctttctcgtggatgctgcctgcatgatacatctttttctgtcctttactttcaacctattttatctctgaattgaaagtgtgttttctataggctgcatataactggatcttgttttctgatccaatataacaatctgtcttttgaatgaattgttaaatctattcacatttaatgttactattgataaggctggactgacttctgccatttaactttttgttttctgtatgtcttgtgtgtttttttgttcctctatatctcctttagtgtttcttttgattaatattttctagtataacattatatctccttcaattatttctttactatatttcccaagttactttctttttttttttttttttttttttttgagacagagtctcgctctgtcacccaggctggagtgcagtggcgcgatctcggttcactgcaagctccgcctcctgggttcacgccattctcctgcctcagcctccccagcagctgggactacaggcgcccgccaccacgcccagctaatttttttgtatttttagtagagacggggtttcaccacgttagccagggtggtctcgatctcctgacctcgtgatccgcccacctcagactcccaaagtgctgggattacaggcgtgagccaccgcgcccagccccaagttactttcttagaagttgctggctggcgcggtggctcatacccgtaatcccagcactttgggaggccaaggtgggtggaccacctgaggtcaggagatcgagaccagcctgactaacatggagaaaccccgtctctactaaaaatacaaaattagctgggcatggtggcacatgcctgtaatcccagctactagagaggctgaggtaggggaatcacttgaacccaggaggcggaggttgcggggagccaagatggtaccactgcacttcagcctgagtaacaagagcgaaactccatatcaaaaaaaaaaaaaaaaaaagaagttgcttaccacatacatcttaatcttttccagattttttttaaaaattagaaataaatatggaatgcatcaggaatttgcatgtcatccttgcacaggggccatgctaatctctgtactgttccaattttggaatatgtgttgctgaagcaagcattttcggatttattctaacttagagctccattccatcttcattctttttgtgctgtaattgtgcatattgtatctatgtatgttaaaagcccaacaatacttgttctaattgttactttaaataattttatgttagagaaactgagaaaaggaatgaagagcaagtatacatttatggagtttttatatttaccctcttattaccattttggttctcctcaccgattcctagggattcaagttgcaatttggtgtcatgatactccattacaactttggtaccacccgttttctttgtacagttattgcaaaatatgtttccatatgttataggttcaacaatataattagatggtttgtgtaagttttttaactgaagaaaaggagaagaaatatgcatttacacaatctcttacaattacagaattacctttacttaaggtgctctttgtttttcatgtggagtcaaattactgtctgaagtcacctgctttcaacctgaagaacttcctttggtatttctcataagtcggtatgctgcttaatttccagatgtttgtctgttactaatgagttctttcagtttttcttttctaagaatgtttttatttcaacttcatctttgaaacatagttttgtaaaatacaagattcttggttgactgttttctttcagcattctgaatatgtcaccctactgccttctgccttccattattcctgatgagcagtcagccactaatcttattggaatatccttgggcatgatgagccatttttctcttactactttcaagattttcttatctttcaggattttttttttttctttttggaggcagggtctcgctctgtcacccaggctgtgattgcagctcactacagcctcatcctcctgggttgaggtgattgtcccatctcagtctcctgagtagctgggattacaggtgtgcactactacccctggttaactttttttttttttttttttttttttcaaagagatggggttttgccacattccccaggctggtcttgaactcctgagctcaagcaatccacccttcttggcttcctaaagtgcagggattacaggtgtgcgccaacatgccaggcctcagcgtttttattaccatgtgtctgcatgtggatctgagtattccgacttggattctttgggcttcttgaatgtgtgcattaatgatttacattacatttgggagattttcagcattatttctttgataatttttctgttactttatctccttctggtactcccattatgcatatcttggtgtgcttaatagtgccccacattattttaaggccctgttttctttattctttcttccctctgttctttagaatgtttaatctctatcaacctattttgctgagtctttcttctgccagttcatatctactgctgagtctttctagtgattttttcatcttgattattatactacttaaaaaaaagttttttttgagacaaagtctcactttgtcacccaggcgcaactgcagtggcaccatctcagcacactgcaacctctgccccgggttcaatcgattctcatgcctcagcaatccaagtagctgggattccaggtgtgcaccaccatggccatctagcttttgtatttttaatagagatggggtttcgccatgttggcctggctgatgtcaaactcctggcctcaagtgctccgcccacctcagcctcccaaagtgctgggattacaggtgtgcaccaccatggccatctaacctttatatttttaatagagatggggttttgccatgttggcctggccaatcttgaactcctggcctcaaatgctctgcccgcctcagcctcccaaagtgctgggattacaggtgtgagccaccacacctggcctgattattatactactcttcaattctagaacttccactgggctcttttcaaatacttcctatctttttattgttattctctatttaacgacacattatcaaatagtcctttacttcttcaagcatagttttagttctttgatcttttttttttttttttttttttgagacagagtctcgcactgtcgcccaggctgaaatgcagtggcaggatctcggctcactgcaacctctgcctcccaggttctcgccattctcctgcctcaggcctccccagtagctgggactacaggcgcctgccaccacgcccagctaattttttgtatttttagtagagacggggtttcaccgtcttagccaggatagtctcgatctcctgacctcgtgatccgcccgcctcagcctcccaaagtgctgggattacaggtgtgagccaacgcgcccagccctttgatcatatttttaatggctgctgttaaatctggtatctggaccctttcacaggaaatttctgttgcctgctttttcttgtgtataggttacacttcctgttttttgtgtgtatgtctcaatttttgttaaaaactggacattttaggctgggtgtggtagctcacgcctgtaatcctagcactttgggaggccaaggtgggtggattgcctgagctcaggagttcaataccagcaactaggtaaaatcctttctctactaaaatacaaaaaattagccgggtgtggtggcatgcacctgtaaccccagctactcgggaggctgagagagaactgcttgaacatgggagacagaggttgcagtgagccgagattgtgccactgccctccaacctgggcgacaagagactccatctcaaaaaaggacattttaagtaatatactgtagcaactctggatactgattccccctccttcagggtttgttgttgttatttgcttatttattagtttagtgacttggttagactattttcgtaaagtctccttccccaaagactggtgaagattttcatgttgtgcctcagagagtgcaaccttgggcatgtacagagtctccctgggatgacattgagccaggctgtctttgactagctctttccatgatctctctgttaagctttctacctcttctggtatcacatccagctgttaggctctactaactgctgactgaattctcgactgtttttgataatgccctggagtatacactgctccaaagtctaattcaattaaattcaggaaggggtaatttttgaaattgagctttgttctgaccttgggagagttcttctcagctgtctccttccctggttctctggtgaactagctggcctgtagatcaccctgttcttaattaagaagagggtacctggccaggcacagtggctcatgcctataaacctggcactctgggaggccaaggagggtggatcacttgaggccaggagtttgagaccagcctgaccaacatggcaaaacccgtctctattcaaaaaagagaaaatataaaaaataaaagagtaaaggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccgggaagcggagcttgcagtgagccgagattgcgccactgcagtccgcagtccgacctgggcgacagagcgagactccgtctcaaaaaaaaaaaaaaaaaaaaaaaaaaagagtaaaaaaaaagaagaggtacctctttttatttgcttaccatcaaaatctcctcaggcatgaacttccctggactctgttcttattaactgcctctctctgtgggcaaattctcagagcagggtcagtagcctctagtcttccgggcttgcctctcccagtgtaaaacctctgtacatgagcaagctagggcaaaggtgatcagagccatagaattcttggcctcaagtatgtgggatagagcttccacaccgtgagtacagtatggagctgggtggaggaaggaagcctctgacctctttgccatactcaccgagaacttagtctcttcaatttggagctggagggaagaagaaaagctgatggccccccactcccaatgagaaacctattccttgtttgggagctgagaattcctgggtttgtatattctgaacctgctggccagagtcaaatgctttaaggctgggcacctaaccaaagatgggccagagttctttaccaggaagctggagctgagactgggagtcttcttatttaataggcagtcttctgcctagtcccatcaaggggaccaggaaaaggtagggtacagtgagagagaagactaagaaacactgtagaaagaaaaagagacgaggagagaagtttctaggttcctaagaatgctctggttactggttcttgttagtccctgagacacagctgtctcaggtctgcaacaaagcccttctccctgaaatcaggtccactttggggagttcaatgaagtttctgtaacaagctgtcaaaagagtcttaatagtcatggtctagaattgccttgaggacaattctactctgagcaaatctcaaattaatccttcttggaaagaagagcaagaatggtcccaggcgcccaaggaaaagctcccgatcactcaggggacccatccttccataccttgattgtgctctgactcctcattggccactcgcatcagggcatctagcaccaaagcattgtctagccatgtgtaccactcttccaactcctgaaggaagctggctgtgcccgttgactctgacacctttcgagtcgccagtttgcaaagccgctcaacaaagccaggaatgctgagaagggccgctgccagggaaagaacaagcgaggaaattttcaaaatagagaccagcaaaactacccctaaagccctaacaaacaatctgcatgataggaataattcttactctgatagtagctaaagctactcttttaaaatgaatgtcaaaccaaggtaaatagtaattatgttacaggcataagctcagtttttagcaggtacagggaaacaggctttttatctactgggaatataaattagaataatctttatgccttaaatgtgtgtgtctttgagccaagaggtcaatttctgggaatttaccataacgaaataaccagtcatattacacgagaatatctgtataaggaggttcactgatgtggtttctgtaacagcaaaaaggtgaaggtaactaaatgttcatcaacagctgattaattaaatacaatgacacatttatacaatggaatactgcatagacactaagaatgatgacacatacatctttatatagtgactcaaaacagctgtttacttgttagagagtaaattattaaacaatatgaatttaattttcttcaaaaatttaaatatttgcttcttcaaaatacacaaggaatgaaagcatatataccaaggaatttactgtggctgttcctaagcaggagagaatacaaacaattctcttaaaattactcatctatagtttctaattttcctgtaacatttactagcaggatgaaagataaacatttttttaaggtaaagaaaaaaactgaataatcacccaatttttctttccaccaatctcattttattcacaatgagtgaggtggaaattctgatcggcatattagaacatcataaactttggaggcagggagatccagattcaaatcctgactccatcatttaaccttctttaaaatgagactaatgcctgatcatgtttgcctgtatatgcattaagtctccctggaaggacagaaaagaagccaagagcagtggctgccacagtaaacagccataagatggaaacagccaaaatgtccgtcaagtggaaaaggggatttaggtcactgagagtaacagtggaactgaaacttttcaataaatacctttttgaatccgcagattttttggaaccatgtgaatgttacacctaattaaaaattaataaataactagatatttcaggatatcaagggattactattaatttttgatacagtggcagttacgtttcttaaaagtcgttatcatgtagaaacactgaaatatatgcaggtgaaattatacgatgtctggggtttacttcaaaataacccaaggtgaagtagggtatagtgaatgggagtagagataaagtaagacaagctggccaggcacggtggctcatgcctgtaatcctagcactttgggaggccaaggtgggtggatcacttgaagctaggagtttaagaccagcctggtcaacatggcgaactccatctctaccaataatacaaaaattagccaggcgtggtatcgtgcgcctgtagtcccagctactcaggaggctgaggcatgagaattgcttgaacctgggatgcagaggttgcagtgagctgagactgcaccactgtactccagcctgggcaagagtgatactctgtctcaacgcgtgcgcacacaaacacacacacacacacacacccctcccataattagtgagtggctataagatgatgtttgaccttagttataaaataaaactacattgaggttcctcaaaaagttggccaggcacggtggctcacgcctgtaatcccagcactttgggaggccgaggcgggtggatcacaaggtcaggagatcgagaccatcctggctaacacggtgaaaccccatctctactaagaagtacaaaaaattagctgggcgcggtggcaggcgcctgtagtcccagctacttgggaggctaaggcaggagaatggcgtgaacccaggaggcagagcttgcagtgagcggagatcgcgccactgcactccggcctgggcgaaagagcgagactctgtctcaaaaaaaaaaaaaaaaaaaaaaagtcaaacataaaattaccatatgacccagcaatttcactcctacatatatacccaaaagaattgaaaacaggtgcacacacaaatatgtgtacacacatgttcatagcagcactattcccaacagccaaaagatgaaaatatcccaaatgtccattaactgaagggataaacaaactgtggtatctacatgatgagtattattcagccatataaaggaatgaagtactgacatgctacaacattaatgaaccacaaaaacattatgccaagtgaaagaaaccagattcagaaggccacatgctatatgattccatttacagttatgcactgcattaacgctgttgcggtcaacaacgaccacacagaagacggtggtgccatcagattataatactgtatttttactataccttttttctatgtttagatacacaaatacttaccattatggtacaactgcctactgtatttagtacagaaacatgctgtacaggtttgcagcctaagtgtatcatgggctataccagctaggtttgtgcaagtacactctatgacattcacacaatgacaaaatcacctcactgcatttctcagaaggatagagactgctaagtgacaacatgactgtatatgaagtaatctgaataggtaaatccacagagaaaaaagcagaatggtggttgccagcagttggggagaaggggtaatgggaagaagctgcttaatgtgtacggcgtttccttttggggtgatgaaaacgtgcaactagatagaggtggtagctgcacaacactgtaaatgtactaaatgccgctgagttgttcactttaagacggctaattttatgttacgtgacttccatctccataaaataattacacagaaatagatttttgtttgagacaccatttttttttttaacatatcagcttggcaaatataaaaaagtttgataatgctctgtactgggaaaggctacagacaacacggtggggaagatgaacaagtaatttctttggagggttaatttgtcaacacttaccaatttccattaatgggggactaattaaattacagtatgcctgtataatagaatattatataattacaaaaaggaatcatataattctatatatactagagtgtaatttttggcaagataagtttaataaaaagagagcagcacttattttatattacctatttacattaccatctgtacaaacaggagtcaatctctctctgtccctccctccttcccccgtcccatgattgggcatgtatagaatatttttcgaaggacgtttaagaaactggtaaaaagttaccacagggaggggaacaaactctctgctgtactttttgtatcatttaaaaaaaattttaggtgtgtattattttttctaaaaacaaatcagtaggcggggcatggtggcccatgcctgtaatcccagcactttgggaggccgaggtgggtggatcacctgagtttgggagttcaagaccagcctggccaatatggcaaaaccgatgtctactaaaaatacaaaaaaattagtcgggcctggtggcgcatgcctgtagtcccagctacttcgcagactgaggcacgagaatcgcttgaaccagaaagtggtggttgcagtaagccaagattgtgccactacactccagcctgggcaacatagccagactgtgtctcaaaataaaacaaagcaaaaaaaacaaaaaagaaataaaaagaatttgtattctacatgaaaatcttcacatagctggatttttactcttatacttctgttctcagcaaaaatagctcctgctgcttgacctgctcctctcaggacagcatgtaggagctagtcttccccattcctgcacactcaacacactcctccagatgggaagtgcccggtactcagggttctaagtctgacttcccacctgagattcttgagctgttgtgagtcactcacctcttgaaaagctaataaaagctaagaccttctccccagaaaaaaattcactggaccaatcatacctagcaactcatacacaatctcatgggctgacatcccagcatatccagtccttgtaattctcacagctttatcagtagctaccacacctggctggttaatgttaaactgacaactaaatcactcacaacccctgagaatttctgcagaagcaggcctcttctctcttctcactgttgaaacatcagtattccccccatactcctgtagtttcccctcctgctttccagcttcatgtgctgcataattcagcccctctcagctaagtgtcacttataaattgacaattatctagcctatagcttcatttacatctctgctaagaatcttgaaagacgttactttcccccagaaggaaaccagctgttctcttctaaatcgacagaagaacaactggtagttctgagaatctcaaaaaggactcaaaaaataattatttattttaaaatgaatctcaaaaaataatgacttacgctatcttcctggggctcagataatttacattagataattacatgtaaattatctgagccccaggaagatagtgtaaatcattatttttgctagacaagaaaatgacctcacagccacagtttattaaaaacgaaacgaaaactttttggcaacatcctacatatttcagctttccatacagttatggactgtgtccccaccccccgcgcccccacaaattcatacgttgaagctctagccctaaatatgactgcgttttgaaacaggccctttagggaatgcggtggtgtgatggttaattttagctgtcaacttgactggactatgggatgcctacatggctggcacagcactgtttctgggtgtgtctgtgaaggtgattccagaagagattggtgtatgagtgtgtgaactaagggaaaatctgccctcaatgtgggcaggcaccatccaattggtgggggagcagtgggaggcgatggaggaaaacaaaaagggattctcccccttctggagtgggaagtttttctcctactgctcttggatatcagactccaggatctttggcttttggtctctgagacttgcaccagcagtttctggggtcctccagccggggagctacaccattagctttcctggttctaagttttccggacttggactgagccatgctaatggcttctcaggttctccagcttgcagacggcttatcttttattttatttatttatttttggagacggagtttcactcgttgcccaggctggagtgcaatggcacaatctcagctcactgcaacctctgcctcctgggttcaagcaattctcctgcttcagcctcccaggttgttgggattacaggcgtgtcaccatgcccagctaatttttgtcgcagatggcttatcatgggactcctccgcctgtgatcgtgtgagccaattcccctaacaaatcccacctcataaatcctactgcttctgtctctctggagactgctgcctaatacagggaagtaattaaggttaaatgaggttacaaagtgtggggccctaaaccgagaggactggtgtccttctaagaagaggaagagacaccagagatctccgtctctcctcaatgacagagaggaaaattcatatgtggatacaataaaaaggtggccttctacaagccaggaagaaaggcctcaaccaggaaccaatcctgacagcaccttgatcttggacttctagcctccagaactgtgagaaaaataaatgtctgttagttaagccacctagtctacggtaccttgttatggcagctcaagctgactgatacacacacctgagataaaggcattcactactggagcgcaattcaagaatgataacattcagaggactaagactttcctcatccactcgtgatttaattatgtccaccagcttggccttcccagggcatttgaatggagaaaatcgtgcgggtaagggaatggagtaggaattatggtcagggttgggaagggtcagatcctgcacaacctcatgtgtggaactacaacttttaggctttatcccaaaagctacaagcagaatcacacatagactgtctactaggctagtcacacagatatgaaagtgaatctagattattttatagagactttaagaaaaaactcccaaataaaaaaacaaaaacaaaaactgggctttccgttctcagttaatcaaaaaagcccagctgaccagaacagttcattcctcaagaataatgggtaatgctggttttacttaagtcagtttacaaataggcatctgaaagcaatcaatactaacaggatcaaaatcatctgccagaatagattccttaaatgggaaactaaaagatgaaagttacttagtgcctcggcatacacaggcatccaatacagaacctttctagccctcagtttcctcacatgtgcaatggggatactaatctctattccacagggcacttgaaggattaaataaaatcacttctaggtaaagcagttacccagcgtttgccacataccaggagcttagcaaatactaccttttgtcaatgtatatggaggtgtgtacaccagctctgaggtataatttagttataatgaaagtcactttttttaaaattatactttaagttctagggtacatgtgcacaacgtgcaggtttgttacatatgtatatatgtgccatgttggaaagtcacatattttttaaaaatgtaaatttcctaaaatgagaactcccccaaaatcataaaggagtgggaagaaaaccgtacacagaccaaggaagaccttgtatctagtgacacaggaggaaaagcaaatctagagagagaataaactggggttggagtccacaaacttctacctttacatatgactacaacatgtaaaattatattccctaataacctacaaagtatagttatgatcacctcatgttcctaactaaaactagacaataacagagtgaatggcaggttcatattcagcctcaggagaggaggctgctctgaagcacttgataactctgcagagccagcctggggaatgcacacaactggactttagaaacaaagaactgttttcagagcatgattagggctacctctgcatggtcatggttttctactgctgctttttaatgatacatttgtataattttaccttgtttatttggtgtaccataaggtacacagtgataggtttttgtattaacaatatttccagcctgttaacattgaaaaactagcttttaagacacaagtttcagttcaaatatgaattatctacactcttatgaccaaaatacatctatgccagtttctccaaaatctgtgcaggcaacaacattttagatagaacacagttcttttgctactgaacagaaataggtgggctagattggttagaatagtgttttgtttttaagtaatatatttacacggttctaaaatcaaacaatatatgaaagtatacagtaaggagtcccactcccactgctactaccttcccttaccagctacttcctattaataaccactttatgtttcttgtgtaccctttgtttctttaggcataccatatatgtgggcaaacgtatggtcttattctccccctgacttaatacaaaaactataccatatacacactgttctgtattttcctctttatcattaatatatcttggagatttttctatagcagtacatagagatcttcattttttcaaagctacacaattttccattatataaatattgaatacactaagcagtccagtgttaatgaacatctgggtttctagtcttttgttattacaaataatgttgcaatgaataatttttttttttttttttgagacggagtctaattatgtcgcccaggctgaagtgcagtggtgcaatctgggctcacagcaacctctgcctcctgggttcaagcaattatcctgcctcagccttctgagtaactgggactacaggcgtgcaccaccacacccagctaatttttgtatttttagtagagatggggtttcgccatgttggccaggctggtctcaaactctgaacctcaagtgatcctcctgcctcggcctcccaaagtgctgggattacaggcgttaagccaccgtgcctggcctcagtgaataatcttgtacagtagtcctcccttagtcaagaccctcacagtggatacctgaaatggcagatagcaccaaaccctatatatactatgttttttcttatatacacacataactatgataaagcttaatttataaattaggtacactaagagattaacaataactaatgataaaatagaacaattgtaacaagatgctgtaataagagttatacgaatgtggtctctctctggaatttttcagttaatattttcggattgcagttgaccacggataactgaaaccacagaaagtgaaactgcagacaagaggagattagtgtctatacatcattatctataaaatgaaggcccaggagagacttagtagatcaaaaagtatatgcatttgtaattctgataaaaagtagttcttaactttttttgtggttctgagcccattaacaaatctaatgaaaggtatggaactcagttcccaaaataatgaacagatacacaaaatttacatactaggagccttcacatggatccatctctagagatatttaaaaggggtgctaggtgtggtggctcacacctgtaattctagcacttcgggaagatgaggtgggtggatcacttgagaccaggaagtcaagaccagcctgggcaacatggcgaaaccccatctctacaaaaaatacaaaaattagcccggtgtggtggcacctgcctatagttccagctactcaggtggctgaggtgggaggatcacctgagcctggggaggtcaaggctgcagtgagccacgattgtgccactgcactcctgcctgggtgacatagtgacgatcccatctcaaaaaaggaaaaaaaaaaaaaaagaaaagaaaagaaaaagaaaaaaaactagatgatttagtaagggcctcagagctataatctcaaaattctgagaacttggagactttaccttgattgatttcagctgcagaaggccccaaactcaagctcttcttctgttgagcatttttggcaagaagcgtgtgcttgtcatcattccctgtatccatctctgaaatggtgacagccagaatccggcagaaattagcgagctgctgctgatcgaaattggatactaaggaactcagattggggacttcatctagtcggatcatttcctacagaagacaaaaatgaaacaatccccatgctcaattcactcaagagattggatgtgcctctctttcgcatccccttacatacggggcttagtggctagatcacttaggttcctatcccagctctgtaacttgcagctctgaaactttggttaagttacaaaaacctttcgagtctttgcttgtttatctgtaaaacggaactaatctacctcacaggattagtctgaggactaaatttaccttttgagcaccactgttttcaattgttcttgtggtatattttgcctatttaaaaaaaaaggaaatgcctaccactcaagttttggaaatcataaattaccattcttgctacagatttgcttttttaaaaaataaaatacttcaaatatagctaaaggcctctgtgtatcccccatccagtcgcatttttttctctccctgcagacgtaaacattattttgaactctgtgtttattattcccgtgcatgtttttatacttacaaatatttccattaaaatacatgttactgctctgcatgcacagtgtgaatgataccatactataccttacttttcatccaacattttgaagtttatctacactgatacaagcagctgattcaatcacttcccgccgttttaccccacagtttaatcatctctctcttttaagagacagggtctcactctgttgctcaagctgcggagcagtagtgtcatcatagcttactgcagactcaaactcctaggcccaagcaatcctcctacctcagcttcccaagtagctaggactacaggtgcgtgtcattacacctggctacttttttaatatttttttttttgagatggtatctcattgtcgcccaggctggagtgcagtggcgcaatcttgactcactgcaaactccacctcctgggttcaagtgattctcctgcctcagcctcccaagtagtggggattacaggcgcccgccaccacgcctggctaatttttgtattttttagtagagatggggtttcaccatgttggccaggctggtcttgaactcctgacctcagatgatccatccaccttggcctcccaaagtgctgggattacaagcgtgagctaccacgcctggccaatactttatttttatggagacagggtctcactattgttgcccaggctggaactcctggactcaagcaatcctcccaccttggcctcctaaactgctgggattacatgtatgagccactgtgcctgaccctttcttcttttaatacatatttaggtaaactacagtttttcactattaaacaatgttgcaatgaacattcttacaggtactctctgtgtacacatgtatgtgaccatttatccagagtgagagcaagaattcttgtctttttcttgactttaccagaaataattccaaaacattaaaattaagttcaatatttgctgtaaattttaatatcctataagttaaattttaataaataccctatatatagtttataagttttccttctattgctagcttttattaagaatgataattttatggaatgccttgtgctatttggttctattctcactcattactccttaaaatctgtaaatggcatgtactacatcaacttttctcattttgaaccatccccgtgctactcagataacacctgctatccttcatcccactaatttacttccactggattgggtgtttctctttcactgcacagatcacatgaacttttaataatctgttcctgcatctatttcccaaataatctgaccatactttgatgaaggcatatatcctaccctttctcctgagactcaatgcacaggacctggtagcaagggtatcagtgaatgtgcccaaaacgagtgcatgattcctagaagctctcatgaatcccatactcgcctctatttctagtaatggaggacaggaatttgctgtgcagggggcattggggaacaactgcttccacttttccttttccctagtacaaaacaaatgtaagctactactattattggcaactgtaacttgtagctcaccagttcctctagccctgtctaactcagtatatggttcccaaaacaggtcccaccacccccagcccacttcttatggttttttgttttgtcttaaattttaagatttttttttttaaggcggagtctcactgtgatgcccaggccagagcgcaatggcacaatctctgctcactgcaacctctgcctcctgggttcaagcaatttttctgtcccagcctcccaagtagctgggactacaggcatgcaccaccatgctgggctaatttttatatttttagtagagacagggtttcaccctgttggccaggctggtctcctgacctcaagtgatctgcccacctcggcctcacaaagtgctgggattacaggcatgagagccactgtggctggccaagaatttacttcttatgttttaatttttgatatgtacaatacgtattatgcagcacaacaaaatgactagctgtgtgtgcctttcctattgcatctctctgcctctgggaaccaccatcattaattattattgccttgccatttaaaaactagttttatcaacaaataatttgcttttttgagctttatgaaaatgatatactacctacagtgttctgcaactggcttctaccactcgattatttcaattactcagctatgttgttgcctatggcaacagttcattcatttctactatataatattccattgtgtgaagaaagtacattcatcccttctcctattcatggatcagagacttggtttcagttttttgttctgaaacaacgaagctacaaatgttcttatgtgtttcttgatgcacaggtggagaagtttttgctggggcagtggcttctaaactttttagctatcaccaagaacgaccaccagtaaacatacatattggaatgtatatagaaatgtcaaacaaaaacttattcaaaaaatacccttacacaatacagtttgatcttttctacttggctggtagacccgatggactgactttacaaccttctgggtttcaatcctaagaatcactgttctaggactgagcttccccacaagtgtgtccaggcacaggttataggtaacccatggcaaaatactgatgcctcagccctcagggtaaagcttccctaggtgctggggcacaagatatgtgcctgttcaactacaaaacaaaaaaaaagaggatattctgctgataaacatttatcagtagtatacaagtgtttccactgatacacacactcaccaacatttgcccactctctccctcactatggtattgatttgcatttccttgattgccaatcaagttaaacatcttctcttttatgtttatggaccatttctttcttgtctgtgaaatgcttgttcataacattgcccatttgtctactgggctatttctctttttcatgctgatttgtaagaattctgtttttttttttttttttgagagggagtcttgctctgttgcccaggctggagtgcagtgacataatcttggctcactgcaacctctgcctcccgggttcaagcaattctcatgccttagcctcccaagtagctgggactacaggtgtgcaccaccatgcctggctaatttttgtattttttagtacaaaatgagactgggttttgccatgatggccaggctgttctcaaactcctgacctcaagagatctgcctgcctcagcctcccaaagtgttggaattacaggcatgagccaccgtgcctggcctgtaagaattattgataaattctgaatacaacaaatcctttgttggttacctgaattcaaatgtcttttcccagtttatggtgtttccactttctttatggtatctatccccctttcaaactgcaccaagatacccataatgtttggtggaacacctcgccattttggggtacacaaaaaggaatgagagggagtgagaggcaagtgggaaaagttgtgaaggagcacttctagactcctatgctgagactgctaaggagtctggagtttccactctggaggtcccctttcacacactggctggaacacacaattactggaataacaacaaagcctttttatcaaagggcacaaagggtctgtctggggcctgcacacatctccaatgtgttcctacagacaataggccacttgtgctgggcaatgacaagcaaggcctggggaaagcacagcaagaagaaatgaacgctgggttcaccacagtgcacatgaacatccacctccctgcaacagcaacctctaggaggctgctcaacaggaagagaaagaaatctgccaaccagcaaccaacatggaggctcaggacagattaggactggggaggtgagctccaggtcctaacaagcattacaccaaaaatgtaacacagtaagtctctaacagagatctatgaatgtatttttgggtatctgtgaattcttgaaatttttaatttagttttcatttcgatcatagctgtttttttttttttttaaagttatgggtcatccgaatattaaccttatgaccaagtggcaggatttcagagctcccatttgtctcctactgcgctggtgccaattaaccaactttaattgttgaagactaatgagaaaaaatagaggcggacttcataagtgaaagccaaacattactaccaaggagaacatatcctaatgaatgaaggcttcacaccagttcagatagcggtaaagtggtaagagaatggagtcactgcatagcatacaatagcacatacatgctacaaagaacttgccccaacggtgatgagtgaggggcaatttgatttcagcaaagtaacatgaggcatcaccctacattcacagagtcaatctgaagttctgagttttagaattagtttgatttcaatgtatgtctgtttaggttttacagtcatttaagaaccactagtataaggggtaatcaagttttatacttgaatacatctgagtaacaagacataatttaacactttcttttaaaaaagctctgtatattattcagttttgagaatccaatggtctgagacacccttccccatctggtcagctagaggacacagctcacctttttaacacccaaaatatcttcaatgagggttgctgtttgtaagaatgtcttcttacttgtcatcaatgtaaacaggaagatcacaaagtcattcttttctgccaaacgctttgtaactccctctgtctgtcacaagaagaaaaggcagaataggttgtaagtacaaaataaattatgaaagtacaaggcatcctttacagacactgaaaagcagtttcacctctgaaaactggcatgagtcaacaaaacaggaatggcatggcttaactgtgggaaacgctcgttaactccccagctatggaagaaaaaagtctcctgacgtgcaggctacacaagtgggaactcacttgtgagaacataacacaatgagatggtatttcctaaaataacttatctcagagttctgaaggccattcttaaatctgaaatcaaacagtgccgaagcatggacttaagagtcagacctaagttctaattccagctgtgtgatcctgagcaaatcacttctcacctttggaactccatttttttcatcagtaaaatgatcatcttaaaagtctttgaggccaggcgtggtggctcacacctgtaaccccagcactttgggaggccaaggtgggcagatcacttgaggccaggagttcgagaccagcctggccaacgtgccaaaaccctgtctactgaaaatataaatattagctgggtgtggtggtgcatgcctgtagtcctagctactcaggaggctgaggtaaaaggatcatttgagcccagaaggtggaggctgcagtgagcctagactgtgcaaatgcactctagccttggcaacagagggagaccctaactcaaaaaaaaaaacaaaacaaaaaaaccaacaacaacaaaaaaacaaaacccccaaggggtatgaaggaagttaaggaaactagctggtaaaagaccttaaaacttaggttcaagagtttggatttaaaactgcagtattgtcaagtgcagtggctcacacctgtaatcccagctaccccggaggctgaggtgagaggaccgcttgaggccaggagtgtgaggccgcagtgagctacaatctgtctcaccactgcattccagcatggggacagagtgaaaccctatctctaaaaagaaagaaactgcagtactgctatagcttacacacacacacacacacacacacacacacacacacatatatatcccacccctactgccttaaggcaactagagcttagtgactttaatatttcttttttttagcctctaagctgaaaaaattccaaggtttactgcgataaacttgacttctccttaaaaagtataatcaagagtgcagacacacacagtttggtaaatgggaaccccaagctaggaggcatttcagaatcagtgaaatgagatatgttgcaacaaatttaattttctttcttttaattttagagacagggtctcactctgttgcccaggctgaggccatgactattcacaagtacgaatatagctcactacagcctcaatcacctgggctcaagcgatccccccacctcagcctcctgaatcactgggactacaggcgtgcaccaccgcacctggcaaatttaattttcaaagtgaaggcatcaataaagatgtgccacgtatataaatctttgacgatcacattaaataggacactaaagaaagcaatctccatctgtattaatacatttcggttcttcagtcgtagcactgacacattaaaaaaaaaaaaaaaaaaggttgttattcccgaactacatacagtacctacactggctatgatatgtgaaacaggactgactctagttttactgacaagcaaaagttctggaagatccaaagaaacagaacaaaaagagaactaaatttacactttctcaaagcttaaaaatctatctccaggctgtcaaatacacaaccctgctgctctaactagctacccttgactacccacctttctcctctcttcaatttatcaaagtgctactccttttacagactgactaaaatgactcctcttggaaaaccttcccaagtcaacagtggccacccaaaaggccatcacaatccttcctttcctctatatcacaagttcttaactgaggccaaaaagcactaaaacaatatataaaaggttgtatgcaactacatttttcgggggagagttttttccatgtaacattcatcataggtttttttttggtttttttttttttttggagacagggtctcactctatagcccaggctgagtgagtacactggcacaatcatggctcaccgcagccttaacctctcaggctcaagcaatcctcccacctcagcctcctgaggagctgggactatggtatataacaccacgtccagccagttttttattttttgtagagacaggttctatgttgcccaggctggtcttgaactcctggacacacacaattctcctgcctcggcctatcaaagtgttgagattataggtgtgagccactgcgcccagcctaatcataagctcttgacctagtatctgtcaagtatctaaatgaggagctacaaactagcatccagtaggccaagctttttctggtgagcagtactgtttatttttttatcttttattttttgagagaccctcactctgtcacccacgctggagtgcagtggtgcaatctcagctcactgcaacctctgcctgctgggttcaagtgattctcttgcctctcctgagtagctgggactataggcatgtaccaccacacctggctaatttttgtgtttttagtacagacggggtttcaccatgttggccaggctggtctggaactcctgacctcaagtgatccacccgcctcagcctcccaaattgctgggattacaggtgatagccaccgtgcccggccaacagtactttttaaacttttgaattcaaacatctttagacaggacatacttgctccagtttagtcacagtttacacaagtccccactccaacaccagcaattccaaacatttcctgttttctgcctgatccctggaggcattggtgtttgcaaccctgagagccccaggttctctacattaacatggaattcagccagatagcacataattgaaccatatcgccctatgttcctcaaattttttaaaaaattttatttaaaaatacttttagttgatatataataaaggtacataatttcagggagcatataatttaatacattcgtataatttataaaaatcaaatcaatgtacttgggatagccatcagcttaaatacttgtcttttctttatgctaagaaccattcaaattctcttctagctattttgaaatgtaaaacagaacagaatattataaactaagtcacccttactgatttatctaacactaggtcttatttatcatcccatatatttgtacccattaatcaacttctcttcatacatttcatttttttaacttaactgttttattggaatgtcactgattaacattaggtatcatgattttaaaacggcctcataaaactagtgactatatggagttgccaaagatggagtcattatattatacactgtcttcttctataaggacaatttcttattggccttagttgcaagaactgtgcatttcctgaacaatttgaaattttatcttatagatctccctgtgcctatctctgtcattggctatacatggctaccgagcacttaaaaggtggccaggccaaactaagatgtgctctgggtgttcaaacaagaggaaagaaaaacaaaacacacacacacattggattttgagagctcaatacgaaaaacaagtaaatatctcattaataattaattatactgattacatgttagaatgacaatattcttgatacactgtgttaaacagaatttatcattaaaattaacttcaccacttcttcctacttttttctttaaaaaaatgtggctacaaggaaatttaaaataacccaagtggcttgcattatatttcaattgcacagcactattatagatgctgaattacgtaagtgttcaaacatattcccctatttgtgttggctgctaagaagcttgaagccaaatttgtgcccatccgtaatacagatgattttatggtcttcatttttatatcagcccaattcctggctatacgttatgtccccacctttatcttcttctttcttacctgcttctaagttcgtgatcttggtagattttgcatacacccacaagatgtcagaaatcttattttgaatgaggcaagaaatgaaatggagctaaatgtttttttttttttatttccacagcatacagggttcacatttataatagcgctcgtctcattctgccatgtattatgattcacagtttacacagtttatcataagtttctaaaagacaggtactgtatcttttaaaaactaacatttatatattgttttacaaagtgttttcacatctactatctcatttctcacaataaatctatgatgttaatacaactgctaactgcattctattggtaagaaactacggcttgggaggtgtactaagccaagggccagaacacaaaatcaggtcctaacactaagtgccatcctcttttgtttctaccagattgctttctttattcaccattcttcctggctcagtggtcaacctagtagagtcctgctcaatatatgggcggggcttctggttctcattttgttttctaacaaggaaaatgaagaaataggcataatggagttaaaatgagtcttaaaggtcacagtctgccaacacttgtcaccttttgacaagtgtccagtttctacttggacagctctaaggccaaaggagtttactacctgctgcagcagtcctggctcttaacaactgctacaaaattctgcctccccgtaatttctaccagttagtcctgattttcaggcacacataaaataagcccgattcccccactgtctgaaagcagctcttatgtctgccattctactcgaatattcccatcttaaacattgcagacctggtaggggagataaacagagccccagtggcaggggtacttacacagacacaggtattatacaggatactcaagcagtccttggacatttcatcacttactgaaagtgaaagggtaatgactggttatttttctcaatgaaaagatagtacaacagttaccacatttttttcagcataagaactgttataatcccctccaagatgattatttttcctttaatatacttcttccagcccctgtctaccaaaggttttcatagtgcccatcagaacctacaagcccatttgcatattgcttttggtttgatacagcagctgttttcttattatgctccacagtcttcataatttcaacctgttgttgtgccatcatttaataaagccattttcccttaatgtcttgcttaggatatatctaggatttgttttttttgttttttgtgttttttggctatttaagtagaccacaaatgaacaacttaatatatatgtgtatccttttcatgtttgtattaaactagttcctcaagatacattttcaaaagtagaactactagaatctaaagtataggcatcttttttttgttcatttgttttggttttttaaatgaggtcttactctgtcactcaggggcacaatcacagctcactgtagtctcgaactcctaggctcaagcaattttccctcctcaccctcccgagtggctaggactgcatgcgcgtgtaccacgcctggctaaaagttaaaagtataagcatctcaattgctcttgttataaatttccattgaaagttctactgcagcaaacaaaaataccatttttatcagactcattaactgagcatttctttttttgttgctgttatctgtgttgggcaaatggctcttaatgtctgatctattactaattcttataaagaaactactacttccctgtgagcaggaacaggaaagtagcataaaaacgaaacttaattttcatgagcccaaaaccaaaagatgatgaaactgagtatgaaccatggacaaagtgctttatttggcacttgcttctattttttaaaattgtttccattcactcctaagctgctaggaatgctaaaatgctaggtctccttctcagatcccaatgggcagccctgccaagctagcttgactagatgggttgtctcataatcctgagacaacgcagacgctctgcatcccttatcaccacctttcaacctactggggctctgtgattgatttatttaacgacttcagagcagggtgaaacactgctctgcatgtctcactatatatctcactatttctagatcctccatttttaaaaggaattgggattcactcattcatcaagtattgttcagcatctactctagagcagatacagtggtgagggaaaaaaagtctcagccttcatggaacctataaatctagtgggaagaagagagactaaacaagtgcaccaacaaataattacaatatgctacttgctatgagggtacatagagaacatgaaagaccaagttagaatacacagctgagacattaaaaatgaatataaatcctcttaataaccactactgcatgcacaaatgaggccttatacagtatgtcgtattggggtaaaaaaaaaatcttttaaattaaaaaaacgcaaagttggtaggagggcattcaccaaacattaactgaagtcatctctggcttttaacaccagagtaaagtgcattcacttttttattttaaattaaataccgaagctgggtgcggtggctcatgcctgtaatcccagcacttgggaggctgaggtgggcagatcacttgaggtcaggagttcgagaccagcctggtcaacatggtgaaaccccatctctactaaaaaaaaaaatacaaaaattagccgggtatggtggtgggcgcctgtaatcccagctacttgagagggtgaggcaggaaaatcacttgagctcaggaggcggaggttgcagtgagccaagatcatgccactgcactccagcctgggcaacagagcgagagactgtctcaaaaaaaaaaaaattgaatacctaaaaagaattatttgtaacgtgtaagttataaaaaataataatagaatattacccatatatctaccacccagcttaagaaatagtgcataccaaaacactctgactttttaccattcacaccctggtaatgcctgaactttttacaaagagcatgtatcacctttctaaaaaaaatagtttaaaaaaaataaaacagagtgatcttactctctcagctagttatatgtgagaaagaagaatttcccaaggaaacatagaagctggatcacaggactatcaaaagtacaaagatgcattctcttaacccagatctgaatttctacgcagtacagaatgaataaagacagctgcttagctgggggaggagagagcaacctgtagctaaatgtaagacatgtccttactattaagagttaggaagggaaggaagaaggcagctgctgggaactgccaccgtatcggcaggttggaaaacaaggaccagtagctaataggtatacttacttttcagtttctgtccccggatagagatcgtaggcctcataagaatttctacaagaagctataaaaaaaattagataaaatcttcaaaattaattcaaagtataacttataaaatgattttgccataaccattcatggcccatgtatctgtcacagtattacaagacccttttgttccaggaggcagttaaggttcagtaaggctcctgactgcagcaagtcaggagacccaggttctagtcccagcttggccacctacttgcccttctctggcctcagctttctcatttttgaaaagaagtattttagattaggtaatctctgggatctaatgcggctttaatttctgaaaagctgcagaacattattactatgattcttctgtttaaagaacatttgtaagtttattttaaaaggactcaaaagatacaccataaattgttaacactgatttctacaggtttgggggttatatgcatgtgttttttattttacatactttaatacaattttgcacaaactgtttaaaataagcatgtatttaaaaattcattctacggagagaaaggatagttgataaaagggtcaatccattagaaagatataacaactacaaatatgtatgtacctaacaatagagcccaaaaatacatgaaacaaaagctggcagaaatgaaggaaacagataatttaaaaataaaagttggacacgttaataactctgctttcaataatagacaactaggcagacgagcaaagaaatagaagacttgaacacaacgaacacatatattctaacagacatctacagaacactccatgcagcaagaacagagtatacattctactcaagtgcacatgaaacattctccaggacagatcaaatttcaggccactgaacaagcctcaataaatttaaaagcacttaaatcacatacagtatgttgtgtggccacaatggaatgaaattaaaatcagaacgaaatttgggaaattcatgaattatatggataattttaaaaacccatcctaaataataatgggccaaagaagaaaccacaaggggaagtataaagtatccaagaaggatgaaaataccaaaacttatgatgtagctaatggagtgctcagagggaaatttatagctgtaaatgcctatattaaaagaaaattcttttttttttttttttttttgagacagagtcttgctttgtctcccaggctggagtgcaatagccctatctcggctcactgcaacctccgcctcctgggttcaagcgattctcctgcctcagcctcagagtagcagggattacaggtgcctgccaccactcctggctactttgtgtatttttagtagaaacgaggttttgccatgttggccaggctggtcttgaactcctgacctcaggtgatctgcccaccttgacctcccaaagtgctgggattacaggcatgagccaccgcacccggccaaagaaaattgtttctaatcgataacctaatcttctaccttaagacactggaaaaagagcagactaaacccaaagcaagcaaaagggagaataagagttgaaacaaatgaaacagagaatagaaaataacagaaaatgaacaaaaccaaaagctggctctttgaagactggcaaattcttaactagactgaccaagaaaaaaggagagaggatacagattactatactcagaaataaaggagagggcattaccaccaaccttatggcaacaaaagtgattataaggaactactatgaacaactgtatgccaacaaattagatgacctagacgaaatggacacattcctagaaagacataaaactaaaaactgacttaagaagaaacagaggctgagcatggttgttcatgcctgtaatcccaagactctgggcggctgaggtgggaggactgattgaagccagaagattgagaccaccctggccaacacagcaagaccccatgtctctacaaataaaataaattagctgggcgtggtggtacgcctatagttccagctacttgggagactgaggcaggaggattacttgagcccagaaggttgaggctgcagtgagctatgatcatgccaatacactccagcctggatggcagggtgagactccatctctttaaaagaaaaaaaagaagaagaagaaggaagaagaagaaagaagaagaaataagaagaagaagaagaggaagaagactacgacgaagaagaagaagacgaagaagacgacgaagacaaagaagatgaagaagaaggcgaagacgaagacaaagatgaagacgaagaagatgaagacaaagaagacgaagatgaagatgaagaaaagatgaagaaaagacgaagacaaagatgaagatgaagaagacgaagacgaagaagacaaagacgaagaagacgaagaagaagaaaaaacagaacatctgaacagccctataacaagtagagactgacttagtgatttaaaaaaaaacaaacaaaaacaacaataacaaaaaactacccataaagaaaaaccttggacatgatggctccattggtgaacaatacaagacatttaaagaatgatcaattcttcacaaactcttccaaaaaacagaagagaaggaaacatttcccaactcattctttgaggcgagtattaccctgataccaaaaccaaacaaagacatcacaagaaaactacagatgagtatctcatgcatatatacacaaaaatcctgaaaaccctagcaaacaaaatccagacacatacaaaaaggattatactccacgcatgatggcttatgcctgtaatcccagcactttgagacgtcaaggcaggaggactacttaagcccaagagtatgcaaccagcctgggcaacatagtgagaccctgtctctacaaaaattaaaaccaaaaattagccaagcatggtagcatgctcctgtagtcccagctactcaggaggccgagataggaggctcgcttgagcccaggagaggctgcagtgagctgtgatcacaccactgcattccagcctgggtgacagagagaccttgtctccaaaaaaaaaaaaaaaaaaaaaaaaacaagaaaaggattatacaccatgacactatgacaaagtggaatttatcccaggaatgtaaggttggttcaacatatgaaaatcaatataatacaccatatcaatagaataaaggacaaaagccaaaagccacacggtcatctcaacagatgcagaaaaagcgtttggaaaaaaaaacccaaaacactttcatgataaaacaatcaacaaggaatagaagagaacttcttcagcctgataaagggcttttattaaaaaacccacagctagcatcacgcttaacagtgaagactaaatgctttcccattaagatgaggaaaaagacaaagatgtccactctcgccacttcaatttaacattgtactgagtgttctaaggcaactagagaaaaataaaaaaagaaattaaaggcacccaaattggaaaggaataagtaaaactaactatttacagatgtcacaatctagaatacagaaaattcctaaagaatccactcaaaaaaactattagagccaataagcaagagttcagcaagatttcacgatatgtgatcaatatacataaatcaactgtatttctaaaattgtagcaagaagcaaaaaaaactggaattatgaaaacaattccatttaaaggagcatcaaaaacagtaaaaacacttagaaataagtttaacaagtgcaagacttgtacaacaaaacatttttgaaagaaattaaagacaatctaaaaaaatggaaagctatcttgtagttcatggatcagaagatttaatattgttaagatgataatactccctaaattgacctacaaattcaatgcaatcactatcaaaatctcagctggcttctttgcagaaattggcaagctgatcctgaaattcacatgcaaatataagggatgcataacagccaaaacaatcttgatctgtaaatacaaaaataaacccgtgtctatgatcagttgatttttgacaaggttgccaagacaattcaatgatgaaagagtagttttttcaataagtggtgctgtgacaactggatagtcacatgcaaaagaatgaaactggacccctgcctcacatgataaacaaagtcaatgcaaatggatcaaagatctaaatgtaagatacaaaatataaaattctcagaagaatacataggtaaatctttgtgaccttggattaggaaatggtttcttagatgcacaccaatacccaagtaacaaaagataaaatgaacttcatcaaaattaaagactttgtgtttcaaaggacaccatcaagagagtgaaaagacagctacttaatgggggaaaatatttgcaaattacaaggaattagtatccagaaactaatacaactcaataataaaaagacaacccaatcaagaaatggagaaagcagatgaacagacatttctccaaaagtccaataataaacagatgatcaacattagtcatcagggaaatgcaaatcaaaaccacaatgagatacctcttcatacctactacgatggttataatgaaaaagacaaatagtaactattggcaaagatgtggagaaattagaaccttcatacactgctggtagatatgtaaaatggtgcagctgcttcagaaagcagtctggcagatccttaaaaattaaacagagttaccagcaactccacttctaggtatataccaagggaaattttttagtaagacaatctaatagaaaatgaaaacacgtccacctaaaaatttatatgccaatgttcatggcagcattattaatcatagcagattactaataatagccaaaagtgggaacaacccaaatgtccatcaactgatgaatggataaataagatgtggtctatccatacagtggaatatttcttgacaataaaaaggaatggagtattgatacacgttatgacacggattaaccttgagaacattatgttaagcaaaagaagccagtcacaaaagaccacgtattgttatgactccatttatgtgaaatgtccagaataattaaatctataaagacagaaagtagattagtggttgcctaaggatgtgggagggaggccttgggaggaaatggggagtggctactgaagaatatggtgtttcttttttggagtgatgaaaatgttttaaaatcaattgtggtgacagtggtatataactctgactatactaaaaaactgaattatatcttttaatatatcttttaaatgggtgacttgtatgacatgaattatatctcaataaagctattatttaaaacaataaaacacctcatgctcagttatagaagctagacataaaaagccacacagtggacgattctgttgatatgaaatatccgtaacaggtgatatggtttggctgtgtgccctcacccaaatctcaccttcaattgtaataatcctcatgtgtcaagggtgggaccaggtggaggtgactggatcatggaggcaggtcccccatgctgttctcgtgataacgagggagtctcatgagatctgatgattttataagtgtctggcatttcccctgcttgcactcactctgtcctgctgccctgtgaagaaggtgcctgcttctcctttgccttctgccatgattgtgagtttcctgaggcctcccaggccacatggaactgtgagtcaattaaacctccttcctttataaactacccagtctcaggtatttcttcattgcagcatgagaacggactgacagaacaggcatattatggacattatggatatttcacaacaggcataatacagacataaaatagattaacggctgccagagctggggagaaggggagatggggagtgactgctaatttgtacaagatttctttttggcgtgataaaaatgttctggttatcactgcacaatactgatgggtggttacaagaattaactttatgtaaattatatttcaataaagctgttattgctggggaggcagggtggctcgtgcctataatcccagctactgaagaaggtgaggcaggaagatcatttgagaccaggagttggagactgcctaaggaacaaagcaagacccccatatctctaacaaatattaaaaaattagccacgtgtgatggcatgaacctgtagtcccagctactcgggagactgaggcaggaggatcactagagcccaggggttcaaggctgcagtgagctaggatcgtaccactgcactccagcctgtgacacagtgcaactctatctctaaaatcatcccaacaatttttaaaagctgtaattaaaaaaaattctgttaaacaaattctaggttaatataaacagaaaaaaataaatgttagaaaggaatactcccgaatcttaacagtggttgaattttggtgacaagtcccaagacagtttttcacattttccttgacaaacaccttttccttttaaaatgagaaaaattaggacctttgttttccaaaaaagtccaattttcacttaaagcatttaaaaattatctatatgctgagaatatgactaagcccatatgtttaaagacattccctatatacgtatgtattttttattattgtttaaatcaagctaggagctaccccagaacaaaaagaaaaattcttccttagccccttctagctataggccaatacttaggcagcatgtccaagacacctcaagccaaatgaagaaggaagattccatagcattttttaaattaaaaagccagcaggcaaatctttgagactagacagttcagcttgagggtctgagaaagcctctgctcatctcaaaccagcaacaaatctttagaaagtaattcacatgccatgagattgctcgtggcatgaatgtgacactataattcaacatcctgaattaagagagatgtgttattttagcttaaagcagcagattaaaaataaaaatcctaaactactacaccacagattgtcaattctagagaagcactgggctcagctttctgcacatctgtatttattgttaactgcacttgcaggagggaaaagaaagaagtctcatgacttgaggcaacaatgaaaactgccctgaacatatgcctgctttgctttgtataatagagacctaaggtcatcctctagaaaaggtgaagtaattattatacagtatataaccattttatagcctgttttcatcatgaattttcccatattactataactttaacattatattaacaacttcataaaattcaacaaaatagatagaataaccattatcttctcctggtagatagtcattacaaataatgttacacctaacggtcttggtatacacaagctttatctcattttgacttacgtcttcatcagagtcacagaaatggaaacaacagggcaaaatgagtagtaatgtttaaagctctgaagtatactacaccaattcttctcaaaagagatcacattcatccaagtaagacgtaagatgctcaagtgcatagaatgtatttaaaataaattaacttcaaatggaagatagactatgtcagtgaataaaagttcctgaatgccaagcaatggcttacacctggcccacaattactgaaaagctggaagatttttacctacaaatctagatttcccataacctctcagggaaacactcaatgggagaggaagagtcatttcctccataaaagcaggcacacactctctagcccaaaacattccccatactcatttaatttatgcagatgagcttgctctgggtctgcttcactccacttagttacatacttcacatctgctttgtaaactctaagacacacttttcacagtcatgttatggatagggtcaggctgacaaggagtgatcccactgagctttgggcagcatgaccaagagaggcttactttgggttcttgctagttattagcagaggaagaaataacaagctgtttggaaaatactaccagcagcaggacagcaagagagttcactaacaaatgcacccaaactgaaggtcagtgaggtgcataattacttaagcaggagcccatgtttataaaatgactgggattcctggagaagctgccctaactcaaagcaggaaagaaaaacccccacaaaatcaagaacagttgatccaaagatgtccgaagatgggataggctcctactagagagccacatttccactgcaaatagacacaggctacagacctacattattatttttcctcccataaatacatcagttcttactaagtagatttcttgatgaaacaattcaaaagtaaagggtctcttgagctagccaagtaagtaaatgccctaggaccaccgacactgctgtggagagggaatgcggagcagcagttaaagaagaaaggctgtggctgatgccatgcatgcagatgtcacactcaatctttactttactggaaaatggtagctttaaacaaaaggaaactttaataattgaagtataacgtgataccgcataaagagtacaatttttttttttgagatggggtcttgctctgtcacccaggctgcagttcatggtgcgatctcggctcactgtagcctcaacctcccggactcaaccaatcctcccacccagcccccatcaagcaactgggactgcaggccatgtgctaccacaatcggctaatttttttaatttttagtagtgatggggttttgccgtgttgcccaggctggtctctaactcctcaactcatgcaatccgcctgcttcggcctcctaaagtgctgggattacaggcatgagtcactgtacctggccatgagtataaattgcacatgtagttttacacactgaacaccattcctgtacaatcgccacatagatcaagataaagactaaccataatacccagaaatgccctctccctcatgttcacttcaggtaatggggttaagggcagatatctaggggagcaaagtcagatcctaacccctcagtcattacaccaaaataaacttcaaatgatgcaaaaatttaaacatacacaataaaaactgtaaaatattagaaggaaataggcatggtttaaatccaacagtgggaaaggcatgaaccaaaactctgaagccattatagtaaaacactgatcaatctgacaatctaaaaattaaacatttctaattttaaaataacatcaaaatttaattctcaaaatagtaaaaaaacacaaatgacaaaggcctttaaaaaatcaaaagcacttacagatcaataagaaaaaggacaaataatcaaatcgaaaaaggtgcaaaacagaaaactggcagaaaaggaactacttataaacacaagaaaaaatatccaatctcagtcatagaagaatgaattttaaaaactatgcagagaatttataaagaactcttacaacacaatattcaaaagagatatatctcaactaagaaattgaaaaaggagagacagacatttctccaaggaagatatacaaatgacaaactcaagaataagatggtcatcattggcaatcagagaaattcaaatcaagaccacaatgagatgtctcttcacactcactagaatggctgtaatcattacagggctggagcgggggctcatgcttgtaatcccagcactttgggaggccaaggctgggggactgtttgagtccgggagtttgagaccagtctgggcaacaaagcaaaacgccatctcagtactttttttttaaattttaaaacactttcaaaatgacagataacaagtgttggcaaggatacagaaaaactgaaacccttatacactgctggtggcaatgtaaaatggcagggcctcttggaaaacattctggcagttcctcaaacaattaaacatagagttaccccatatgacccagcaactgtattcctaggtatgtatatgcctgagagaaatgaaaacatgtccacaaagaacctgtgcaagaatgtttatagcactgttattcataatagcaaaaagacggaggcaatccaaatgcctctcaacaaataaaaaatgtggcatatccacactgtgaaacatcatttgaccataaaaatgagtgttgatacatgctacaacacagatgaatctagaaatcattacgctaagtgaaaagaagccagtcacaaaagaccacatattatataactaaatttatacaaaacgtccacgacagagaaatctatagagacaaaaagtatactagtgtggggagagggggaggaggtggtagaggtaggggagtgctagataaaacacatggagtttcttttttgaggtgatgaaaatgctctaaagttgactgtggtgatggttgcacatatctgcaaatatactaagaaccactgaattgtgtacttcaaatgggtgaaatgtatggtatatgaaccatatcttaataaagctgattaaaaaaaactatggaaaaatatcacttttctcataactgcttagtaaatacttaaaatacactgtttgtggaagggggcttctgggtactggtaatgttctatttcctaatttgggtggtagtgcaaaagtgaatgtattttgtggtaattattggtgtacttttctgtatgaaggttatacttcagtaacaaagtctataaaaatacacacataaacacagtatgttggtgatggtatgggtaaacagacgctcatactttgttcctggggatataaactgacaaaacttcttaggtaggtaaacttcagatttataaacaataaaaaacatacgttcttgttccaaaggtatatactcacatgtatacaaaaatatgtaaaagatctttactgcaaccttgtctgtaagagaaatggtgtggaaaaattataaatgccagtcaactggggcctgggtaaataaattatggcacaatcacaccaggaaacactaaccatttaatttttttttgagagctcatcacccaggctagagtgcagtggtatgatcttggctcacggcaacctctgcctcccggcttcaagcaattctcctgcctcagcctcctgagtagctgggattacaagtgcctgccaccgtgcccatctaattttggtatttttagtagagacagggtttcaccatcttggacaggttggtctcgaactcctgacctcacgatccacctgccttggcctcccaaagtgctgggattacaggcgtgagccactgcgcccggcctaaaattgttttaattaaaaaaaaaaaaagaagaagaaagaaggtacagaatagtgtagccgtgtatatggtatatcactctatgtgcggagaaaataaactaaggtggaggtggtggggaatatttacacacacacacacacacacacacacacacacaaaaaaaacaaaacatcttaagcaggacacacaagaaactagtaatcacaactggttctgaggaggggacactgggaaatttagagatgtacttttcacttagtatttttttaaaaaggtatacaccaatttaaataaatgaattcttctgaagtaaaaactatgctgcttcaacatgtaaatgccgctaagcttctaaaagttaaaatctgaaatgttataaattctgaaatctttcctatctcccatttcaaaagttaaattcccaaaccattaagcagtagtaacagcagtagtaataataaatagctacttacatcaacacctccaaacaagtcaaaaatgtaagtatttggataagtggtttcttgggtaagtttcctctcttcagtaacaaatgccatagcctccatggagagaagaggagaaatttcctagtttttttaaaaaaaagtacatacattgttttagtaaccaacacagaccagacactaaagcatcagtttgagctttagtcccaactggagttcccatgaacacaaagacagtccccaatgaaccacacttcctagtattcaggcccctgtaaagccccttctcacactaaatctgggctggtatgacacacctcaactaacagcatgtggtagaagtgacactaggccacttccagcacaagccttaagaaagccaggctcacacctgtaatcccagcactttgggaggctaaggtgggcggattacttgaggccaggagtttgagaccagcctggccaacatggcaaaaccccatctctactaaaagtacaaaaattagccggatgtggtgctacacacctgtaatcccagctactcaggaatttgaagtacaagaagcacttgagccacggaggcagacgttgcagtgagccaacatagagccactgcactccagcctcggcaacagagggagactctgtcattaaaaaaaaataaaaagcctgacagacttcgctcttgccatcctggaacccaaacatcatgctgtgagaacacagccaatggggaggccataaggagaagaaatgaggccaagagcaacatccttagttgatctccagctgacagccagcaccaacctgccagggatgtgagagagaccattttggacattccagatatctcagtaccaccctacccaacagcacatgaagcagaggagccactcagtcaaccaagaattgttgacagataaatgattcttgttacttccggctgctaggtttcggggtgactagttctgtagcaataaccaagtcactgctcaaatataatctcctgcaggaagtcttcttgatttcccccaaccagaagcatgctcttcagacccccaaaacaatctacaactcatttgtaacatttagcacttttactttgcacctatttacaaaccatttctttttttttttttgagagggagtctcgctctgccacccaggctggagtgcagtggcgcgatctcggctcactgcaagctctgcctcccgggttcacgccattctcctgcctcagcctcccgagtagctgggactacaggcgcccgccactgcgcccggctaattttttgtattttcagtagagacggggtttcaccgtggtctccatctcctaaccttgtgatccacccgcctcggcctcccagagtgctgggattacaggcgtgagtcactgcgcccggccaaacctatgttatttcttccctagactaaacgttccttgaggacaacacaaacagatcccactttgtattccccacagattccaagacagagtatttgatacagcacacatagggtagtagttctcaaactgacggccacagaatcctttgttcatatggaacaaagctaagaggaaaaaaaatcttacaagaaaacctaaaacatagtaagtttcaaataagtacttacttaatgaataaagaaatgatgatcacatagcaaagatatacaaatattaatttccctcccctttaacttcacctcaatctcctgagattttgcaagaagcaaataatgtgcaactctttgaaaagcatttttaaacaagtttaagaagctataattatttctatcagactgtgagattcaaaatatgaaaatgtctcttcttaaacgtaaagactaagtttaattagtttctattacattttaaaataacttactatgtgatataataatcaaaacttcacctctgaagaacactgtcattcagtttcattacgttcaatataacttacatggtttttatttatttatttttgctgttttttattttattatactttaagttctagggtacttgtgcacaacatgcaggtttgttacatatgtatacatgtgccatgttggtgcgctgtacccattaactcgtcatttatattaggtatatctcctaatgctatccctcccccctccccccaccccatgccaggccctggtgtgtgacgttccccaccctgtgtccaagtgttctcattgctcaattcccacctatgagtgagaacacacggtgtttggttttctgtccttaacgatagtttgctcagaatgatggtttccagattcatccatgtccctacaaaggacatgaactcatcattttttatggctgcatagtactccatggtgtatatgtgccacattttcttaatccagcctaccattaaatggacatttgggttggttccaagtcttttgctattgtgaatagtgctgcaataaatatacacgtgcatgtgtctttatagcagcatgatttataatcctttgggtaaatacccagtaatgggatggctgggtcaaatggtatttctagttctagatccttgaggaatcgccacactgtcttccacaatggttgaactagtttacagtcccaccaacagtttaaaagtgttcctatttctccacatcctctccagcacctgttgtttcctgactttttaataatcgccattctaactagtgtgagatggtatctcattgtggttttgatttgcatttctctgatggccagtgatgatgagcattttttcctgtgtctgttggctgcataaacgtcttcttttgaaaagtgtctgttcatatccttcgcccagtttttgatggggctgtttgcttttttcttgtaaatttaagttctttgtagattctggatattagccctttgtcagatgggtagattgcaaaaattttctcccattccgtaggttgcctgttcactctgatggtagtttcttttgctatgcagaagctctttagtttaattagatcccatttgccaattctggcttttgttgccattgcttttggtgttttagtcatgaagtccttgcccgtgcctatggcctgaatagtattgcctagattttcttctagggtttttatggttttaggtctaacatttaagtctttaatcaatcttgaattaatttttgtataaagtgtaaggaaaggatccagtttcagctttctacatgtggctagccagttttcccagcactatttattaaatagggaatcctttccccatttcttgcttttgtcaggtttgtcaaagatcggacagttgtagatatgcggcattatttctgagggctctgttctgttccactggtctatatctctgttttggtaatagtaccatgctgttttggttacagtagccttgtagtatagtttgaagtcaggtagcgtgatgcctccagctttggttcttttggcttaggattgtcttggcaatgtgggctcttttttggttccatatgaactttaaagtagttttttccaattctgtgaagaaagtaattggtagcttgatggggatggcattgaatctatcaattacctcgggcaatatggccattttcacaatattgattcttcctattcatgagcatgggatgttcttccatttgtttgtgtcctcttttatttcattgagcagtggtttgtagttcttcttgaagaggtccttcacatcccttgtgagttggattcctaagtattttagtctctttgtagcaattgtgaatgggagttcactcatgatttggctgtctgttattggtgtataggaatgcttgcgacttttgcacattgattttgtatcctgagactttgctgaagctgctcatcagtttaagaggattttgggctgagacaatggggttttctaaatatacaatcatgtcatctgcaaacagggacaatttgacttcctcttttcctaattgaataccctttatttctttctcctgcctgacttccctggccaaaacttccgacactatgttgaataggagtgtgctgagagagggcacccctgtcttgtgccagttttcaaagggaatgcttccagtttttgcccattcagtatgatattggctgtgggtctgtcataaatagctcctattattttaagatacgtcccatcaatacctagtttattgagagttttcagcatgaagggctgttgaattttgtcgaaggccttttctgcacctattatcatgtggtttttgtctttggttctgtttatatgatggattacgtttattgatttgcatatgttgaaccagccttgtatcccagggatgaagccaacttgatcgtggtgaataagctttttgatgtgctgctggatttggtttgccagtattttattgaggatttttgcattgatgttcatcagggatattggtctaaaattctcttttgttatgtctctgccaggctttggtatcaggatgatgctggcctcataaaatgagttagggaggattccctctttttctattgattggaatagcttcagaaggaatggtaccagctcctctttgtacctctggtagaattcggctgtgaatccgtctggtcctggactttttttggttggtaggctattaattattgcctcaacttcagagcctgttattggtctattcagggattcattcaacttcttcccagtttagtcttgggagggtgtatgtgtccaggaatttatccatttcttctagattttctagtttatttccgtagaggtgtttatagtattctctgatggtagtttgtatttctgtgggattggtggtaatatcccctttatcattttttattgcatctatttgattcttctctcttttcttattagtcttgctagcggtctttcaattttgttgatcttttcgaaaaacgagcttctggattcattgattttttgaagggttttttgtgtctctatatccttcagtactgctctgatcttagttatttctcgccttctgctagcttttgaatgtgtttgctcttgcttttctagttcttttaattgtgatgttagggtgtcaattttagatctttccggctttctcttgtgtgcatttagtgctataaatttccctctacacactgctttaaatgtgtcccagagatcctggtatgtcatgtctttgttctcaatggtttcaaagaacacctttatttctgccttcatttcgttatgtacccagcagtcattcaggagcaggttgttcagtttccacgtagttgagtggttttgagtgagtttcttaatcctgagttctagtttgattgcactgttgtctgagagacagtttgttataatttctgttcttttacatttactaaggagtgctttacttccaactatgtggtcaattttggaataagtgcgacgtggcgctgagaagaatgtatattctgttgatttggggtggagagttctgtagatgtctattaggtccgcttggtgcagagctgagttcaattcctggatatccctgttaactttctgtctcgttgatctgtctaatgttgacagtggggtgttaaagtctcccattattattgtgtgggagtctaagtttctttgtaggacttgctttatgaacctgggtgctcctgtattgggtacatacatatttaggatagttaactcttcttgttgaattgatccctttaccattatgtaatggccttgtctcttttgatctttgttggtttaaagtctgttttatcagagactaggattgcaacccctgcttttttttttgttttccacttgcttggtagatcttcctccatccctttattttgagcctatgtgtgtctctgcatgtgagatgggtctcctgaatacagcacgctgatgggtctagactctttatccaatttgccagtctgtcttttaactggagcatttagcccatttacatttaaggttaattttgttatgtgtgaatttgatcctgtcattatgatgttagctggttattttgctcattagttgatgcagtttcttcctagcatcgaaggtctttacaatttggcatgtttttgcagtagttggtatcggttgttcctttccatgtttagcgcttccttcaggagctcttgtaaggcaggcctggtggtgacaaaatctctcagcatttgcttgtctgtaaaggattttacttctccttcacttatgaagcttagtttggctggatatgaaactctgggttgaaaattcttttctttaagaatgttgaatattggcccccactctcttctggcttgtagagtttctgccaagagaaccgctgttagtctgatgggcttccctttgtgggtaacccgacctttctctctggctgcccttaatattttttccttaatttcaactttggtgaatctgacaattatgtgtcttggagttcctctcctcgaggagtatctttgtggcgttctctgtatttcctgaatttgaatgttgacctgccttgctagattggggaagttctcctggataatatcctgaagagtgttttccaacttggttccattctccccgtcactttcaggtacaccaatcagacgtagatttggtcttttcacacagtcccatatttcttggaggctttgtttgtttctttttattcttttttctctaaacttctcttctcgcttcatttcattcatttgatcttcaatcactgataccctttcttccacttgatcgaattggctactgaagcttgtgcatgcgtcacgtagttttcgagccatggtgttcagctccatcaggtcatttaaggtcttttctatgctgtttattctagttagccattcgtctaatcttttgtcaaggtttttagcttctttatgatgggttcaaacatcctcctttagctcggagaagtttgttattactgatcatctgaagccttctctcaactcatcaaagtcattctccatccagctttgttccgttgctggtgaggagctgtgttcctttggaggagaagaggcgctctgatttttagaattttcagcttttctgctctggtttctccctatctttgtggttttatctacctttggtctttgatgatggtgacatacagatggggttttggtgtggatgtcctttctgtttgttagttttccttctaacagtcaggaccctcagctgcaggtctgtcagagtttgctggaggtccactccagaccctgtttgtctgggtatcaccagcagaggctgcagaacagcaaatattgcagaacggcaaatgctgctgcctgatcctttctctggaagcttcatctcagaggggcacccggctgtatgaggtgtcagtcggcccctactgggaggtgtctcccagttaggctactcaggggtcagggacccacttgaggaggcagtctgtctgttctcagatctcaaactctgtgctgggagaaccactactctcttcaaagctgtcagacagggacatttaaacctgcagaagtttctgctgccttttgttcagctatgccctgctcccagaggtggagtctacagaggcaggcaggtctccttgagctgcggtgggctccactcagttcgagcttcccggccgctttgtttacctacgcaagtctcagcagttgtggacacccctccccctgctttgctgccaccttgcagtttgatctcagactgctgtgctagcagtgagcaaggctccgtgggcatgggaccctccaagccaggcgcaggatataatctcctgctgtgccatttgctaaggccgttggaaaagtgcagttattagggtgggagtgtcccgattttctgggtaccatctgtcatggcttcccttggctaggaaagggaattcccccaccccttgcgactcccaggtgaggcaatgccccgccctgcttcagctcacactccgtggactgcacccactgtctgacaagccccagtgagatgaacccggtacctcagttggaaatgcaaaaatcacctgtcttaggcatcgctcatgctgggtaccatagactggagctgttcctatttggccatcctggaaccggacccccacttacatgggttttaactgcattagagctggtgctctaaattcaaatttagatccaaactacagctaaagtttagatactccagccctactgaatcagaatttcaggggtggggcccagaactctgtgttttaacacgctcttcaggtgattcataaacacactccagtttgagaacaattatcctagcataatgtctccgtgctctttccactcatttcccaatcatcaaacctggccccttcaatcttcttacgttagtaataatattcaacaaccataaggcctcttccacgagccttcagcagtatgtaatcttctaaacccttttcaaaaagaatgtcatcccaatcacaggatagtgttgacctcccaaaattaagcaagcactgccacaaaataaacacacatagagcccaaaaggataacacagggaacctaatggtgattatcttgggatgatgggattataggtgatgtatattttcttctttgtactttttctgaatttctcaaattttctatagctcacacagtaaaaacaggctatttttaaaagccaacggaagggaaaaaaaaaacagcagacatcttgtgtcacctagaggccctgaatacgcccctccaaggtccttaagagttaccttgaggatgttttgacactcaacaaagtcactgtggaggtggctggtggtgtgcagcttgaggagcagctgaggaattccactccacttggattgtttgtccctctccgtcaaaaaagtctcagtgaactgtgagtcaaaaaaagagagaacatattattgtattattgatgataatagctgatacggcaaaggagagcagccgacttccaaacccacccaggacagttacaaatttatctctaagcactactataaaaagcctctttctataaatcttcaaaacctccccaacagcctattactatgttataataagaaggagggattggaaatactcttgtaccactcacaaacagcacaatatcattgagagtggacttagactcgtcgtaaatatatactggaaattctagggcaactgctaaaagtatttttacaagaagaataattgatatgctaagagatgaaaaataatggaaccatataaactgctcagttaagcccataaaaggcagaaaaagaggggaagacaaagaacaagtataataaaacaaacatggtatacattaatccaactatatcaataatcactgtaaatatctatgaatggtctaaacttaccaatcaaaggaaagagactaaaagaatggatttaaaaaaccaagatccaacaatatgctgtctacaagaaactcatttaaatatacatatagataggttaaaagtaagggaaaaggccaggcacggtggctcacacctgtaatcccaacactttgggaagccgaggcaggtggatcacctgaagtcaggagtttgagaccagcctggctaacgtgataaaaccccatctctactaaaaatacaaaaattagctgagtgtggtggcaagtgcctgtaatcccagctactcgggaggctgaggcaggagaaccgcttgaactcgggaggcagaggttgcagtgagccaagatcgcgccatggcaccccagcctgggcgacagaccaaaactctgtctcaaaaaatagaaaaggggatggagaaagataaatcacataaaaagaaagatggagtagttatattaatttcagagaaagcaggcttcataaaaaaattatcagaaataaagtgggtcatttcataatgacaaaggggttaattctccaagaagacataacaatccttatcatatatgcacctaacaacagcatgtcaaaatatgaggcaaaaactgcctaaaagaactgccagacaaaaaaatggcaaatcaactcttacagttaaaatattttaaactaaatgaaaatataacttatcaaaatttgtgggatgcagcgaaagcagtaataagccagaagtgtatatcactgaatgcatataatagaaaagaagaaatatctatagtcaataatctaagcttctaccttagtaaactacagaaagagcagtttaagcctacagcaaacagaagaaaaaaataaaaatgagagcaaacatcactgaaattgaaaacaaaaaatcaagagagaaaacaaaaccaaaagttagttctttaaaaagatcaatacgattgataaactctaaccaggcttatcaagaaaaaaagacagaaacacaaattactaatatgtggtacataagaggagttattattactgactccatggacttcgaaggatgataaaggagtattatgaaccgtttgccccaatatttgcctagcactttgggaggctaaggcgggaggattgcttcagctcaggagttcaagaccagcctgagcaacatagcgagagctcgcctctactaaaaataaaaaaaaaaaataagccaagggtggtggtgcacacctctggtctcagctactcagaaggatgaggtgagaagatcacttgagcttgggagaatgaggcggcagtgagctatgacagcaccactgcactccagcctgggtgacagagcaagataaaaaaaaaaaaaaaaaaagacatcattataaccaaagagtgaaacaactcaaatgtccatcaactgatgaataaataaaatatagtatatccatccaatggaatattattcagcaaaaaaagtaatgatgtactgatactacaacaaggataaatcttgagaccattatgctaaatcaaagaagccagtgtctaactgggcacagtgtctcatgcctctgatgccaaagtaatcccagcactttgggaggccaaggtggacagatcacttgagctcacaagtacaagaccagcctgagcaacatggcgaaaccccacctctacaaaaaaaaaaaaaattagccggatgtagtggcacatgcctgtagtcccagctactcgggaggctgaggtgggagaatggcttgagcccaggaggcagaggttgcagtgagccaagactgcgccactgcacttcagcctgggcgataaagccagaccttgtctcaaaacagaaacaaaaacaaagcgagactccatctaaaaaaaaaaaaaaaaaaaaaacctaaaagaagccagtctcaaaaggccatatattgtctgattctattaatttaaaatgtccagaataagcatatccagagacagaaagatcactggttggcaaaggctgtggggatggggaaactggggagtaactgctactgaataccaggtttcttttttgggtgataaaatattctaaaattgatcgtgatgatagatgcataactttgtgcatacagtaaaaaccactgacttgtacacttcagttttttttttttttacccttttatctccttagtttaccaacttgtacactttaaaagggtgacttgtgcaatatgtgaattacatctcaataaagctgctataaaaactaaataaataaatgaacataagtgaactttttaggatgacaatgttctaaaaccagattgtggtgatggttgcataacttcatcaatttgctgaaaatcactgaactgtacacttacaatgggtaaattttattgtatataaatcatgcctcaataaagttgtttaagaaatatttaaaagaagaaagaagagaaaagggaacaaaggataggacattaaaaaagcatatattgtattaagcaagtataaacaccaaaatgtcagttattacattaaatctaaatatgctaaaatgctccaattaaaaacaaagtttgggccggttacagggcctcatgcatgttactgcagcactttgggaggcagagacagacagatcacttgagcccaggagttaaagaccagcctaggcaacacagtgaaaccctgtctctaccaaaggaaaaaaaaaaaaattaggcatggcagcatgtgcctgtaatcccagttactcaggtagctgaggcaggaggatcccttgatgctaggaggccgagactgcagtgacctgtgatctggtaccatactccagcccgggcaacagaatgcaaccctgtctcaaaaaataaatagaaaataaaataaaataaaaataaaataaaataaaataaaaacaaagtttgttatgccacctaccagagaaacagaaaggctaaaagtaaaagaatgggaaataatactcaatgtaaacatataccaaaaggtagtataactatattaaaatcagcaagagcaaagaaaaagcattaccaagccaggcatggtggctcacacctctaatcccagcactctgggagaccgagacaggtagatcacttaggtcaggagttcaagaccagactggccaacatgatgaaaccctgtctccactaaaaatgcaaaattagctgggcatggtggcacaggcctgtaatcccagttactcgagaggctgaggcaggagaattgcttgaacccaggaggtggaggttgcagtgagccaagaatgcaccattgcactccagcctgggtgacagagtgagactctgtctcaaaaacaaaacaaaacaaaaacaacaacaacaacaaagtactaccagagataggcactttaataaggataaaaatggcaaactggataatgttaatgatcctaaatttgaaaatatctaataaaaacttcaacatacataaagcaaaaactgacaaggctaaaaggagaaataaggaaatccataatcataatgggagattttacacatctctttcaaaaactgatatatgcagacaaaaattagtgagaatacagaatattttaacacaattaaacaaaatggacacacctcgagcagtatatctagtaaatgcactctacatgtcctttacaaatacaaatagaacatttgcaaaatgaccagctgctaggccatactgcaaatgtcaatacatttaaatgaactgaaatacagactatgttccacatcagaaagatagttataaaatccccatgtttggaaaataatatacttctcaaaaatgcatgggtcaaagaagaaatcgaagtggatatatgaaaatatcctgaaagataatgaaaatactacacatccaaactgatgaggtacaactaaagccatgcttactgaccattataaccctgataccaaaacctgtcaaaaggaaaactgcaggccagtttcactaacgacgatgtaagggaaaaaccacaattcaaaaattgaattaaatttaaaatttgtatcagaaattaaaagttggtttaatatttgaaaattttattcaatgcaattcaccacattgataacataaaggagaataattatttgataacataaaggagaataatgatatgaatacctcaattgatacagaaaaagcatttgataaaatttaacatccattcatgataaactcaaataaaaaaatgaatttctagtaacaggtatccacagaaaacccactgcaaacatcatacttaatggcaaaatgtttaaggttttttccctcttagatcaggaacatgctacttctattcatcatactggaggtctcaggcagcagagtaaggcaagatatatagaagacataagattggtaagggtaagataaaagattcatttgcagatgatataatattcacagatgactgtacagtatacaatccaaaagaacctaaatgtaaattatcaactattacagcaatatacatgaataatataaaaatctatcttatttttatattacagctacaatttttaaatgcagttttaaaaacactacttacaataccacttttaaaaacgctaaggaataaatctaacaaagttatgcaagacggctacatagaaaactgtaacacactcttggctgggcacagtggttcacacttgtaatcccagcactttgggaggctaaggtgggcagatcacctgagatcaagagtttgagtctagcctgaccaacatggtgaaaccccatctctactaaaaataccaaaaaaaaaaaaattagctgggcgtgatggtgggcgcctgtaatcccagctactaggggaggctgaggcaggagaatcgcttgaacctgggaggcggaggttgcagaaagctgagatggtgccactgcactacaatctgggagacagagcgagactccatctcaaaaaaaaaaaaaattacaggaaaataacttccaatttatacttctatacccagtcaaactaaatatatgtgtcagaataagacattttctgatctgcaagctttaaaaaaaaaaaaaaagttacctcccatgtactctttcttgggaggctaccagagagtgtgccccaggaaaaaggaagacaattgaggaaacaaggtctaaaacatgagaaaagaaagaaaaatgctctggatttggtgaagagaagtcccaagaactcagctgtatagcagacctagtaagcagctaaactaaactgctaaactgctaaactgcagtaggggaatgaagggcttcaaatgatttctttaaaaaaaaaaaaaagttttagaatttaggaacaaattataagtatacagaaagcaggccgggcgcggtggctcacacctgtaatcccagcactttgggaggccgaggtgggtggattacctgaggtcaggagttcgagaccagcctggccaacatagtgaaaccccatctctactaaaaatacaaaaaaattagccagacttggtggcgggcacctgtaatcccagctactcggggggcataggttgcagggagccgagattgcgccattccactccagccgggcaacagtgcgagactccgtcccaaaaaaaaaaaaggtatacacaaggcaaaacaatttaaaaggatgcattatacctgacttcaccaccatgcgatacaccaatgtagcaaaatttcacttgtaccccattaatacaaatttaaaaacagtaaaataaattaaaggatgcactattatgctaatgaaagtcaaaagggtatatgtatatatgtgtgtatatatgtatatatgtgtatatatgtatatatctatatatgtatatacgtatatatgtgtatatatgtatatatgtatatgtatatatgtttatatgcatatatgtgtatatgtatatatgtttatatgcatatatgtgtatatgtatatatgtttatatgcatatatgtgtatatgtatgtatgtttatatgcatatatgtgtatatgtatatatgtttatatgcatatatgtgtatatgtatatatgtttatatgcatatatgtgtatatgtatatatgtttatatgcatatatatgtatatgtatatatatacagggtatatgtatatttatccttcaatttaaaaaatggaagaggactgtttttaacactacaaaaaaattatataaaacaaatataattgtacaatatatggccctgctgtaaataataatgctaacatcataatgtaaatacttaacaacagccaaagctgtgatataactacattggtagactagtggagacgagtaagaaggaatcgaatgtgtgaattaaatcctagtctttcaagagcgaagaaaatagatagtatctaaaagtgaaaagaaacagctttataaccatgttatttagaaatactggctctcctgtttcctggatccttctcttgcaaaagaaatagctaagtatggacactgaacagcattaggagtatggaacttagaggtgcaaagcaggcagggcactgctgtttttttgttacaagctccatgatacgcagagatctaatcacagtccaaatatttgacttcacaagcaatttctttactatttatacattgtcttacaaggcatgtactctcatttaaaaataataatttggggctgggcacagcggcttacactcatatcctagcacttgggaggccaaggtaggcccattgcttgatcccaggagtttaagaccagcctgggcaacatggcagaaccttatctctacaaaaaaatacaaaaatgagctgcgtatggtggcatgcgcctatagtcccagatactcgggaggctgaggtgggaggattgcttgagcccaggaggcggaggtttcagtagttttttttcaccactacactccagcctgggcaacagagcaagaccctgtctaaaaaaaaaaaaaaaaatcattagaattttgttttccttgtgaacagctcagcactgtgacaaaagtgccaccattcagtcatcttcccagttgaattactggcaggtatcacatgggttttctcttgaagtacttgtggtttaacagtttacagcagtgataatctcttacatcataccctctaaactgtcaacctgtttagcaaatatgacatttctagacatttacagtttccactgattttattgtttttaaaaataactaaaggctaaattccatgaagataactgtgcatcttatttgtttctttaatcctaaatgtaagacatctaatgcctcaaaataccacactaaatgcaagggtctcgcgcaatctcactgcaacctccgtctcctgggctcaagtgatcctcccacctcagccctgcccccttccccctcccagccccccacaccaccaccactgccaccaagtagctgggattacaggcacgcaccaccacgcccggataattttttgtatttttagtaaagatggagtttcacccatcttgcccaggttggtcttgaactcctgagcttaaagaatccgtctagctcggcctcccaaagtgctagaattacaggtgtgagccaccgcacccagcctaaatagatatcttaaaggataataaagataaaataccttgttgggttattctgacaatacatgttaaccactgaatacttttgtgtcctttaatggggtatgattcagtaaaattaggagcaaatttaagggcttgtgaatacgaaaacttcccattgaaatgaatggcatatgtgtgtgtgtgtgtgtgtgtgtgtgtgtgtgtgtgtgtgtgtgtatgtgtgtgtatatatatatgtgtatatatatgtgtgtgtgtgtatatatgtgtgtgtgtgtgtgtgtgtgtgtgtgtgtgtgtgtgtgtgtgtgtgtgtatatatatatgaataagactttatcctaatcaggagtttttcagtatcaaattacctttgaaaagccaggtaagatttgtttgacttaaatgcctaggtttcataaatactaatggttagaatgaggctaaaacaagtagtgcctaatgaaattttttttaaaagttagtgaatctggctgggcacggcgactcacatctgtaatcccagcactttgggaggctgaggtgggtggatcaagagatcaagagtttgagaccagcctagccagcatggtgaaaccctgtctttactaaaaatacaaaaagtagccgggcatggtggcacatgcctatagtcccagctactcaggaggctgaggcaggggaatcgcttgaacccgggaagcggaggttgcagtgagccgagactgtgcactgcactccagcctgggcaacagcacgagactccatctcacaaaaaaaagaagtcagccaggcgcagtggctcacacctgtaatcccagcaccttgggaggccgaggtgggcggatcatgaggtcaggagatcgagaccatcctggctaacacggtgaaaccccgtctctactaaaaatacaaaaaaaaaaaaaaaattagccaggcgtggtggcgggcacctgtagtcccagctactcgggaggctgaggcaggagaatggcatgaacctgggaggcggagcttgcagtgagctgagattgcaccattgccctccagcctgggcaacagagcgagactccatctcaaaaaaaaaaaaaaagtcagtgaatctaaagaaaaaatataaagtaaggaacactcaggggcatttagatatgccaataatcataaaggaagcaacgactgccatgtgggaaatataacagcatcccacaacccagtctgtcaccagattgccaaactgaagggcatccagggatggcgggtcatgacctgtaatgtgagctgcactatcctagagcccagcgtttactcagtctatcctttccttcaactcgtactgatcatttggggccttgtacgcccatggtaaggaatccagaattgattctatcaactgcaatgagatgaggctggagagttttaagcaaaggatttataagatctgatttgcattaagcatcaaatgtgctgatgaatatgaaaaggctcagtaaactccaaagtatcaccccagcatattttattgccttgacagagggcagaacaaaacgaagggaagaaaataaagcaaactaaaatccaagtagaattgacccattacaaattatgtggccagtagtcatgactcaggcttcccttatgccccatttagggagatggagaagatcaggtctgatggaaaagggagaggattttggctttcgacatactaagtttgagatgcccattagacatcaaagtggaaatgctagaaggcaattggatatacctacccatccagagctaaaaggagaaacacgaatggaaacagaaacttaggatccgtcagcgtatagagcagttattaataacaatacaagctaatctttatagcagtaagctatgtacttatagctgagaaagtatgtttcagtcactattttaagtgctttatatgtacaactcacttggtcctcaccacaaccttggaaggtcaggtcctatgatacttactttacaaataaataaactgaggcataaacagattaaataacttgcctaaagacacatagtaagtaaataccagagctaggtttcaaacctaggtaatctgactctggaactctttaaagccatgagattgcttggcaagtacagacagaaattagactgaggacacagtatttcaatttaagcaatttcctgaatatcaaatggctggcaaggtttcaggctctgcagatacaatgaaaaataagacacagtctcttcaggaagcccagtctaattaactactagctgagagcaactatttactaaacaactattcctatatgccacactggctacgcttttaaaacatattatttcacttaatcctcctaacaactttatgggatagatgttatcatcacctctttagaaataaggaaactgaagttaagagaggttaaataacttaatcttggtcaccctgctaggattcaattcaggtccaccttactttaaaatctgatttttttttccaaggaaattccatcacatgctataccacagatgatccttgaggacattatgttaagtaaaataagccagtcacaaaaagacaaatactgtattactatactggggttatctaaagtagtcaaactcacaaaaacagaaaggagaagaatggttaccaaggactgggagggagaaatgggaagttgttcaatggtagtttcagatttgcaagatgaaaaagttctggaaatctgtttcacaacaatgggaatatacttcacattacttaactatacaccttttttttttaagatacagggtttcaccatgttgcccaggctggtctcaaactcctgggctcaaacaatccgctcacctcagcttcccaaagtgttgggattacaggtgtaagccaccacactgggcctgacctatacacttataaatggttaagatggtaaattttatgtttacatatatatatttatatatgtatatagtttgaggcagggtctcattctgttgcccaggctgcagtgtaatggtgtgatcatggctcactgcagccttgacttgccaggctcaagcgatcttcccacctcgggttcctgagcagctgggactacaggcatgcatttccacacctggttaatgttttttttttttttttttgtagtagagctggggctccaccacactgcccaggcttgactcaaactggctcaggtgatcatcccatcttggtctcccaaagtgctgggattacacacatgagccaatgtacctggcctatattatatatttgtttactacaatgaaaaaattttggctaggcgcagtggctcacacctgtaatcccagcactttgggaggacaaggcagggggatcatgacgtcaggagttggagaccagcctgaccgacatggtgaaaccccatctctactaaaaatacaaaaagtagccaggtgtggtggcacacgactgtaatcccagctactcaggaggctgaggcagaattgcttgaatccgggagacggaggatgcagtgaaccgagattgcgccactgcactccagtctgggcaacagagcgagactccgtctaaaaaataaaaaataaaaaatgtttaaatctggtcttttcctactataccacatgccataacagtataaaaatatgggtaatctgggcaacagagcaaaaccctgtctctaccaaagatacaaaaattaatggggtgtggtggcacacgcctgtagtcccagctactagggaggctaaggtgggaggatcacttgagcccaggaagttgagcttgccactgcactccagcctgggtgacagagcaagattctgtctcaaaaaaaaaaaagggtaagcaaataacttattctgcaggatgggagagaggagcggaagggcacagcatggaataaattaaaaaaaaaaaaaactaagaaaagaaatatttgaaagatgaaggtccccattttatcctcactatagctccaggcagggggtctctacttcaacttctgaaatagctcccacacctatatacaaccatcagttaccacaataacctgccttgcttttgtggctccagaatattccagcagccttgtatctggtgagctttttttttttttttttgagatgaagtctcactatgtcacccaggctggagtgcagtggcgtgatctcagctcacaacctccacctcccaggttcaagcaattctcctgcctcagcctccagagtagctgggattacaggtgcttaccaccacgcccggctaatttttgtatttttagtagagacggggcttcgccatgttggccaggctggtctcaaactcctgacctcagatgatccacccgcctcgacctcccaaagcactgggattacaggaatgagccactgtgcccagcctctggtgggcttttctaggctcctcccattctaagtcattcagcactggaaacaaattaagttgcctaaattgcctcatttatcatgctaaagaacttctgtggctccctattattctcagaacactgattatcaggatgggtgtttaggaacatatcatctaacctctctgagcctcagtttcctcatccatattaagaaatgagtcttgaagcataactcaaaagcttgaggattaaagataacatatgccttgtaccgtactttttctaaaggttatgtgctcactaaaagttagtcctctttctgcctgtctctacacacccagctcggtaaggtatcttagcttaagaaggaaagagcaagtatttctaggatttttttttttttttgagacagggtctcactctgtcaatctccgcctcccaggctcaggtaatcctcccacctcagcctccccacaggcacacaccaccaccatgcccggctaaatttttggagacacaggattttgccgtgttgcctgggctggtctcgaattcctggattcaagcgatctgcctgcctcggcctcccaaagtgctgggattacagggtcttaagtcaggtcttaagaacacaagggggaaaaaggctgctcatctatggttgtgtgatggccaggaaaacatttctcactcacaactgctcctttcgtccacagtcctccatacacacagaccccctagcctttattttattctatccttttcgtcatttgaggaagtgatattcaaattaaatttaaataaagttacactgtgaaaagtctcactcccattttctccccctttagcccaattcccacctcactcccaccaaccactttatcagtttcttgagtatccttcaaactttttatgaaaaagctgtctttaaataaagctaatgttttacaggtgttagcaaggttcggggtgggggagggtggaagaagtagtggactaggtcctattcaacctggtttaccctaaagcagtgtccttcgctcactcaaaaataacaataatcatatgttgggcacctgttataagtcagatgcttaatattaactaccttcagcccttagtatgcaccagaaatgcactaatgctttacacaattgccccctttgatctccacaacaatcttaaaaggtaggttactaattctcatcttcatcttacaagtgaggaaactgaggcacagaaaggtcacctgcccaagcaagagagaggagcagggctttgaacccaggcctgactccagagtccctcttaaccgtgacgctctacactagcaaaccagatatagtctctgcacccgctggaactcagcctaatgtgggagacgggcgagaaaaaggcagacaacaaagaaatacgaaattatagactaagaaagtaatgtaaacgaaacgaattcgcggctgtgacagaataacaatggggacctactttagaaaggggaaggaaagagtgtcaaggaaaggcctgagaaaatgacatttatgaaatgctcgggaaccatttgggggctgcaatgatgaccccctcccccacgcccaatacacgggataaactcgaggcccacagaggctaaaacaaacacgacagggtcccccagacgtgtagacgtggcgcaaagaagggaggatccagaaactgggcccagggttcgtacacgccatcagtgtccacagcagcccagccccgctcggcccctgacaagggacatcaagccagctgcttcccacggccccgctgcagctgagagcgtccgggcagctcacagggaggccttccggccagccaaaagggctttggcccgtccagcggcggcctggaaaggcctcttccccggccccccgccagctcccgcctggtccgccccgccccgcccctcctggtccagcctcgtacctgcaccgcccggaccaggccccggccggtcagctggccctgccgcagctgcagcagaatgttaccaggccgcggccggccgccccatccgccccaagccgccactgtggctgccgaccgtctccctcggccggctccagacccagccgctaccggcgccgccgccatgtctcctcgtcggacaaacaggaagcaagcggcc

SEQ ID NO: 2 - DNA, 20 bp, Artificial Sequence
  Synthetic Primer
ccacagccagtttccttctc

SEQ ID NO: 3 - DNA, 22 bp, Artificial Sequence
  Synthetic Primer
tttcctgtagctcctctggttc

SEQ ID NO: 4 - DNA, 20 bp, Artificial Sequence
  Synthetic Primer
cttgtgggtagggagtgagg

SEQ ID NO: 5 - DNA, 20 bp, Artificial Sequence
  Synthetic Primer
accagggagctgttgatctg

SEQ ID NO: 6 - DNA, 20 bp, Artificial Sequence
  Synthetic Primer
gcagatgctgaatgcttcct

SEQ ID NO: 7 - DNA, 21 bp, Artificial Sequence
  Synthetic Primer
gaaagtgggtttgtgtgtgct

SEQ ID NO: 8 - DNA, 20 bp, Artificial Sequence
  Synthetic Primer
ttatgccctggctttctcaa

SEQ ID NO: 9 - DNA, 20 bp, Artificial Sequence
  Synthetic Primer
tgtcccttccttcaatacgg

SEQ ID NO: 10 - DNA, 24 bp, Artificial Sequence
  Synthetic Primer
cagttctctgttagtccttgttgg

SEQ ID NO: 11 - DNA, 20 bp, Artificial Sequence
  Synthetic Primer
ctgccttggcctctcaaagt

SEQ ID NO: 12 - DNA, 20 bp, Artificial Sequence
  Synthetic Primer
aaagaagccaagagcagtgg

SEQ ID NO: 13 - DNA, 20 bp, Artificial Sequence
  Synthetic Primer
ggactacaggcgcacgatac

SEQ ID NO: 14 - DNA, 20 bp, Artificial Sequence
  Synthetic Primer
aaaagcagaatggtggttgc

SEQ ID NO: 15 - DNA, 24 bp, Artificial Sequence
  Synthetic Primer
ttgactcctgtttgtacagatggt

SEQ ID NO: 16 - DNA, 20 bp, Artificial Sequence
  Synthetic Primer
agtgaatgtgcccaaaacga

SEQ ID NO: 17 - DNA, 20 bp, Artificial Sequence
  Synthetic Primer
cagggctagaggaactggtg

SEQ ID NO: 18 - DNA, 21 bp, Artificial Sequence
  Synthetic Primer
cactgctctgcatgtctcact

SEQ ID NO: 19 - DNA, 20 bp, Artificial Sequence
  Synthetic Primer
tttggtgaatgccctcctac

SEQ ID NO: 20 - DNA, 20 bp, Artificial Sequence
  Synthetic Primer
ggttggaaaacaaggaccag

SEQ ID NO: 21 - DNA, 21 bp, Artificial Sequence
  Synthetic Primer
ccccaaacctgtagaaatcag

SEQ ID NO: 22 - DNA, 38 bp, Artificial Sequence
  Synthetic Primer
tgtaaaacgacggccagttccagcctcgtacctgcacc

SEQ ID NO: 23 - DNA, 38 bp, Artificial Sequence
  Synthetic Primer
caggaaacagctatgaccagcaggcaggaagcggactc

SEQ ID NO: 24 - DNA, 43 bp, Artificial Sequence
  Synthetic Primer
tgtaaaacgacggccagtaaagaatgtcatcccaatcacagga

SEQ ID NO: 25 - DNA, 45 bp, Artificial Sequence
  Synthetic Primer
caggaaacagctatgacccctttgccgtatcagctattatcatca

SEQ ID NO: 26 - DNA, 40 bp, Artificial Sequence
  Synthetic Primer
tgtaaaacgacggccagttgcttcaacatgtaaatgccgc

SEQ ID NO: 27 - DNA, 40 bp, Artificial Sequence
  Synthetic Primer
caggaaacagctatgaccttcatgggaactccagttggga

SEQ ID NO: 28 - DNA, 42 bp, Artificial Sequence
  Synthetic Primer
tgtaaaacgacggccagttcccaaggaaacatagaagctgga

SEQ ID NO: 29 - DNA, 39 bp, Artificial Sequence
  Synthetic Primer
caggaaacagctatgaccggccatgaatggttatggcaa

SEQ ID NO: 30 - DNA, 38 bp, Artificial Sequence
  Synthetic Primer
tgtaaaacgacggccagtcagctgctgggaactgccac

SEQ ID NO: 31 - DNA, 38 bp, Artificial Sequence
  Synthetic Primer
caggaaacagctatgaccgggcaagtaggtggccaagc

SEQ ID NO: 32 - DNA, 39 bp, Artificial Sequence
  Synthetic Primer
tgtaaaacgacggccagtgctgcagcagtcctggctctt

SEQ ID NO: 33 - DNA, 40 bp, Artificial Sequence
  Synthetic Primer
caggaaacagctatgaccgggcttgtaggttctgatgggc

SEQ ID NO: 34 - DNA, 39 bp, Artificial Sequence
  Synthetic Primer
tgtaaaacgacggccagtcccaacggtgatgagtgaggg

SEQ ID NO: 35 - DNA, 39 bp, Artificial Sequence
  Synthetic Primer
caggaaacagctatgacctcccacagttaagccatgcca

SEQ ID NO: 36 - DNA, 44 bp, Artificial Sequence
  Synthetic Primer
tgtaaaacgacggccagtaagggcctcagagctataatctcaaa

SEQ ID NO: 37 - DNA, 39 bp, Artificial Sequence
  Synthetic Primer
caggaaacagctatgacctgcgaaagagaggcacatcca

SEQ ID NO: 38 - DNA, 38 bp, Artificial Sequence
  Synthetic Primer
tgtaaaacgacggccagtagcaagaatggtcccaggcg

SEQ ID NO: 39 - DNA, 40 bp, Artificial Sequence
  Synthetic Primer
caggaaacagctatgacctcccagaaattgacctcttggc

SEQ ID NO: 40 - DNA, 38 bp, Artificial Sequence
  Synthetic Primer
tgtaaaacgacggccagttcccggttctggaatcagcc

SEQ ID NO: 41 - DNA, 38 bp, Artificial Sequence
  Synthetic Primer
caggaaacagctatgacctctctgcgctcacctggctc

SEQ ID NO: 42 - DNA, 38 bp, Artificial Sequence
  Synthetic Primer
tgtaaaacgacggccagttcaagtgatctgcccgctcg

SEQ ID NO: 43 - DNA, 38 bp, Artificial Sequence
  Synthetic Primer
caggaaacagctatgacctgggtttgtgtgtgctgggc

SEQ ID NO: 44 - DNA, 38 bp, Artificial Sequence
  Synthetic Primer
tgtaaaacgacggccagtgccacgctgtccactcttgc

SEQ ID NO: 45 - DNA, 43 bp, Artificial Sequence
  Synthetic Primer
caggaaacagctatgacctgctgttgatcagatgtggaagtga

SEQ ID NO: 46 - DNA, 38 bp, Artificial Sequence
  Synthetic Primer
tgtaaaacgacggccagtgaatgcacgagacaaggcgg

SEQ ID NO: 47 - DNA, 38 bp, Artificial Sequence
  Synthetic Primer
caggaaacagctatgacctccacaggaagtgggcagga

SEQ ID NO: 48 - DNA, 38 bp, Artificial Sequence
  Synthetic Primer
tgtaaaacgacggccagtaattcatggcagggcccgta

SEQ ID NO: 49 - DNA, 38 bp, Artificial Sequence
  Synthetic Primer
caggaaacagctatgacccccagttggagcaggaaggc

SEQ ID NO: 50 - DNA, 38 bp, Artificial Sequence
  Synthetic Primer
tgtaaaacgacggccagtgcctgcacaggcatttggaa

SEQ ID NO: 51 - DNA, 38 bp, Artificial Sequence
  Synthetic Primer
caggaaacagctatgacccgtccgcttcccacacacat

SEQ ID NO: 52 - DNA, 38 bp, Artificial Sequence
  Synthetic Primer
tgtaaaacgacggccagtacaagccacccattcccacc

SEQ ID NO: 53 - DNA, 38 bp, Artificial Sequence
  Synthetic Primer
caggaaacagctatgaccaggcctggtcctgtccttgg

SEQ ID NO: 54 - DNA, 38 bp, Artificial Sequence
  Synthetic Primer
tgtaaaacgacggccagtaagccaattgccctggaagc

SEQ ID NO: 55 - DNA, 40 bp, Artificial Sequence
  Synthetic Primer
caggaaacagctatgacccctggtgtgttaggctcaccga

SEQ ID NO: 56 - DNA, 38 bp, Artificial Sequence
  Synthetic Primer
tgtaaaacgacggccagtcacttgcgagccctccttcc

SEQ ID NO: 57 - DNA, 38 bp, Artificial Sequence
  Synthetic Primer
caggaaacagctatgaccggtgctcctgggcactctga

SEQ ID NO: 58 - DNA, 38 bp, Artificial Sequence
  Synthetic Primer
tgtaaaacgacggccagtagcagagggtgactgccggt

SEQ ID NO: 59 - DNA, 38 bp, Artificial Sequence
  Synthetic Primer
caggaaacagctatgaccgggagagctctgtgggtggc

SEQ ID NO: 60 - DNA, 38 bp, Artificial Sequence
  Synthetic Primer
tgtaaaacgacggccagttgaaggaaaggtgggcatgg

SEQ ID NO: 61 - DNA, 38 bp, Artificial Sequence
  Synthetic Primer
caggaaacagctatgaccttccacaacctgctgcgctt